(19) United States
(12) Patent Application Publication
(10) Pub. No.: US 2019/0142001 A1
(43) Pub. Date: May 16, 2019

(54) COMPOSITE MATERIALS CONTAINING STRUCTURAL POLYSACCHARIDES AND STRUCTURAL PROTEINS AND FORMED FROM IONIC LIQUID COMPOSITIONS

(71) Applicant: Marquette University, Milwaukee, WI (US)

(72) Inventor: Chieu D. Tran, Fox Point, WI (US)

(73) Assignee: Marquette University, Milwaukee, WI (US)

(21) Appl. No.: 16/124,841

(22) Filed: Sep. 7, 2018

Related U.S. Application Data
(63) Continuation-in-part of application No. PCT/US2017/021552, filed on Mar. 9, 2017.
(60) Provisional application No. 62/305,757, filed on Mar. 9, 2016.

(51) Int. Cl. A01N 43/16; A01N 59/16; A01N 59/20; A01N 37/18; A61L 15/44; A61L 15/22; A61L 15/18

(52) U.S. Cl. CPC A01N 43/16; A01N 59/16; A01N 59/20; A61L 2300/404; A61L 15/44; A61L 15/225; A61L 15/18; A01N 37/18

(57) ABSTRACT

Disclosed herein are composite materials, ionic liquid compositions for preparing the composite materials, and methods for using the composite materials prepared from the ionic liquid compositions. The composite materials typically include structural polysaccharides, structural proteins, and optionally including metal or metal oxide particles. The composite materials may be prepared from ionic liquid compositions comprising the structural polysaccharides, structural proteins, and the optional metal or metal oxide particles, where the ionic liquid is removed from the ionic liquid compositions to obtain the composite materials.

### CROSS-REFERENCE TO RELATED PATENT APPLICATIONS

The present application is a continuation-in-part of International Application PCT/US2017/021552, filed on Mar. 9, 2017, and published on Sep. 14, 2017 as WO 2017/156256, which application claims the benefit of priority under 35 U.S.C. § 119(e) to U.S. Provisional Application No. 62/305,757, filed on Mar. 9, 2016, the contents of which are incorporated herein by reference in their entireties.

### STATEMENT REGARDING FEDERALLY SPONSORED RESEARCH OR DEVELOPMENT

This invention was made with government support under R15GM099033 awarded by the National Institutes of Health. The government has certain rights in the invention.

### BACKGROUND

The field of the invention relates to composite materials containing structural polysaccharides and/or structural proteins and ionic liquid composition for preparing the composite materials. Optionally, the composite materials may include metal or metal oxide particles. In particular, the field of the invention relates to composite materials containing structural polysaccharides, such as cellulose, chitin, or chitosan, and/or structural proteins, such as keratin, and optionally containing metal or metal oxide particles, such as gold, silver, or copper particles or oxide particles thereof, which composite materials are formed from ionic liquid compositions.

### SUMMARY

Disclosed herein are composite materials comprising one or more structural polysaccharides and/or one or more structural proteins. The composite materials may be prepared from ionic liquid compositions comprising the one or more polysaccharides and/or one or more proteins dissolved in the one or more ionic liquids forming liquid ionic compositions. Optionally, the composite materials comprise one or more metal and/or metal oxide particles.

The composite materials may be prepared from ionic liquid compositions comprising the one or more polysaccharides and/or one or more proteins dissolved in the one or more ionic liquids forming liquid ionic compositions. Optionally, one or more metal and/or metal oxide particles are added to the one or more ionic liquid compositions, for example, as metal salts which subsequently are reduced in situ. The composite materials may be prepared from the ionic liquid compositions, for example, by removing the ionic liquid from the ionic liquid composition and retaining the one or more structural polysaccharides, the one or more structural proteins, and the optional one or more metal and/or metal oxide particles.

The disclosed composites and liquid compositions may comprise one or more structural polysaccharides, which may include, but are not limited to polymers such as polysaccharides comprising monosaccharides linked via beta-1,4 linkages. For example, structural polysaccharides may include polymers of 6-carbon monosaccharides linked via beta-1,4 linkages. Suitable structural polysaccharides for the disclosed compositions may include, but are not limited to cellulose, chitin, and modified forms of chitin such as chitosan.

The disclosed composites may include any suitable concentration of the structural polysaccharide(s) for example, where the composites comprises at least about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, 95%, or 100% (w/w) of the structural polysaccharide(s), or the composite comprises less than about 100%, 95%, 90%80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.5% (w/w) of the structural polysaccharide(s), or the composite comprises a concentration of the structural polysaccharide(s) within a range bounded by end-points selected from any of the foregoing percentage concentrations (e.g., 5-25% (w/w)).

The disclosed composites may comprise one or more structural proteins. Suitable structural proteins may include, but are not limited to, keratin. Natural components that comprise keratin and may be used to prepare the disclosed composite materials include, but are not limited to, wool, hair, and/or feathers.

The disclosed composites may include any suitable concentration of the structural protein(s) for example, where the composites comprises at least about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, 95%, or 100% (w/w) of the structural protein(s), or the composite comprises less than about 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.5% (w/w) of the structural protein(s), or the composite comprises a concentration of the structural protein(s) within a range bounded by end-points selected from any of the foregoing percentage concentrations (e.g., 2-10% (w/w)).

The disclosed composites may comprise a selected ratio concentration of structural polysaccharide(s) to structural protein(s). For example, the composites may comprise a percentage (w/w) of the structural polysaccharide(s) to percentage (w/w) of the structural protein(s) at a ratio selected from 100:0, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, or 0:100, or a ratio range bounded by any of the foregoing ratios as end points for the ratio range (e.g., 40:60 to 60:40 as a ratio range).

The disclosed composite materials may be formed from ionic liquid compositions, for example, ionic liquid compositions comprising the one or more polysaccharides and/or the one or more proteins dissolved in one or more ionic liquids to form an ionic liquid composition. Optionally, one or more metal and/or metal oxide particles are added to the ionic liquid composition (e.g., as metal salts which subsequently are reduced).

Suitable ionic liquids for forming the ionic liquid compositions may include but are not limited to alkylated imidazolium salts. In some embodiments, the alkylated imidazolium salt is selected from a group consisting of 1-butyl-3-methylimidazolium salt, 1-ethyl-3-methylimidazolium salt, and 1-allyl-3-methylimidazolium salt. Suitable salts may include, but are not limited to chloride salts.

In the disclosed ionic liquid compositions, a structural polysaccharide may be dissolved in an ionic liquid. In some embodiments, the ionic liquid may comprise at least about 2%, 4%, 6%, 8%, 10%, 15%, 20% w/w, dissolved structural polysaccharide, or a percentage range bounded by any of the foregoing percentages as end points for the percentage range (e.g., 6% to 15%).

In the disclosed ionic liquid compositions, a structural protein may be dissolved in the ionic liquid. In some embodiments, the ionic liquid may comprises at least about 2%, 4%, 6%, 8%, 10%, 15%, 20% w/w, dissolved structural protein, or a percentage range bounded by any of the foregoing percentages as end points for the percentage range (e.g., 2% to 10%).

The disclosed ionic liquid compositions may be utilized in methods for preparing the disclosed composite materials that comprise a structural polysaccharide, a structural protein, and/or optionally metal and/or metal oxide particles. For example, in the disclosed methods, a composite material comprising a structural polysaccharide, a structural protein, and optionally a metal and/or metal oxide particles may be prepared by: (1) obtaining or preparing one or more ionic liquid compositions as disclosed herein comprising a structural polysaccharide and/or a structural protein, where the structural polysaccharide and/or the structural protein are dissolved in one or more ionic liquids to form an ionic liquid composition; optionally (2) adding a metal salt to the ionic liquid composition and optionally reducing the metal salt in situ, and (3) removing the ionic liquid from the ionic liquid composition; and (4) retaining the structural polysaccharide, the structural protein, and the optional metal and/or metal oxide salt in the form of particles. The ionic liquid may be removed from the compositions by steps that include, but are not limited to washing (e.g., with an aqueous solution). The water remaining in the composite materials after washing may be removed from the composite materials by steps that include, but are not limited to drying (e.g., in air) and lyophilizing (i.e., drying under a vacuum). The composite material may be formed into any desirable shape, for example, a film or a powder (e.g., a powder of microparticles and/or particles).

The disclosed composite materials may be utilized in a variety of processes. In some embodiments, the composite materials may be utilized to remove a contaminant from a stream (e.g., a liquid stream or a gas stream). As such, the methods may include contacting the stream with the composite material and optionally passing the stream through the composite material. Contaminants may include, but are not limited to, chlorophenols (e.g., 2-chlorophenol, 3-chlorophenol, 4-chlorophenol, 3,4-dichlorophenol, and 2,4,5-triochlorophenol), bisphenol A, 2,4,6-trichloroanisole (e.g., as “cork taint” in wine), 1-methylocyclopropene, and metal ions (e.g., Cd²⁺, Pb²⁺, and Zn²⁺).

In other embodiments, the composite materials may be utilized to remove toxins from an aqueous environment, for example, as part of a filter treatment or as part of a batch treatment. For example, the composite material may be contacted with toxins in water whereby the toxins have an affinity for the composite material and the toxins are incorporated into the composite material thereby removing the toxins from the water. Toxins removed by the disclosed methods may include any toxins that have an affinity for the composite material, which may include bacterial toxins such as microcystins which are produced by cyanobacteria. After the composite material has been utilized to remove toxins from the aqueous environment, the composite material may be regenerated by treating the composite material in order to remove the toxins from the composite material and enable the composite material to be reused again (i.e., via regeneration of the composite's capacity for adsorbing toxins).

In other embodiments, the composite material may be utilized to purify a compound (e.g., from an aqueous solution, a liquid stream, or a gas stream). For example, the composite material may be utilized to purify a compound from an aqueous solution, a liquid stream, or a gas stream that comprises the compound by contacting the aqueous solution, the liquid stream, or the gas stream with the composite material where the composite material has an affinity for the compound to be purified. In some embodiments, the compound may be purified from a mixture of compounds in an aqueous solution, a liquid stream, or a gas stream, for example where the composite material had a greater affinity for the compound to be purified than for the other compounds in the mixture. The composite material may be contacted with the aqueous solution, the liquid stream, or the gas stream comprising the mixture of compounds in order to bind preferentially the compound to be purified to the composite material and remove the compound from the mixture of compounds in the aqueous solution, the liquid stream, or the gas stream. In some embodiments, the compound to be purified is a specific enantiomer of the compound present in a racemic mixture of the compound, for example, where the composite material has a greater affinity for one enantiomer of the compound versus another enantiomer of the compound.

In other embodiments, the composite materials may be utilized to kill or eliminate microbes, including but not limited to bacteria and/or fungi. For example, the composite material may be contacted with bacteria including but not limited to Staphylococcus aureus (including methicillin-resistant strains i.e., MRSA), and Enterococcus faecalis (including vancomycin-resistant strains i.e., VRE), Pseudomonas aeruginosa, Escherichia coli, in order to kill or eliminate the bacteria. For example, the composite material may be contacted with fungi including but not limited to Candida species such as Candida albicans. The bacteria and/or fungi killed or eliminated in the disclosed methods may be present in an aqueous solution, a liquid stream, or a gas stream as contemplated herein.

In other embodiments, the composite material may be utilized to inhibit the attachment and biofilm formation in water of various microbes including but not limited to bacteria (such as Pseudomonas aeruginosa, Escherichia coli, Staphylococcus aureus, methicillin resistant S. aureus and vancomycin resistant Enterococcus faecalis) and/or fungi. For example, where a substrate is utilized in an aqueous environment, the substrate may be coated with the composite material in order to inhibit or prevent bacterial growth and/or fungal growth and biofilm formation on the substrate.

In other embodiments, the composite materials may be utilized for preparing a wound dressing or a bandage. For example, the composite materials may be utilized for preparing a wound dressing or a bandage for a wound where the composite material is in contact with the wound and promotes healing and inhibits growth of bacteria and/or fungi and/or kills bacteria and/or fungi. In some embodiments, the composite materials may further comprise a therapeutic agent, which may include but is not limited to, an anti-microbial agent (e.g., an anti-bacterial agent (such as an anti-biotic) and/or anti-fungal agent and/or an anti-viral agent).

Preferably, the composite material is biocompatible. For example, preferably the composite material is compatible with fibroblast adherence and viability, in particular, where the composite material is utilized as a wound dressing or as a bandage for a wound.

Preferably, the composite material exhibits anti-inflammatory activity. For example, preferably, the composite material inhibits production of pro-inflammatory cytokines such as interleukin-6 (IL-6) by immune cells such as macrophages. Optionally, an anti-inflammatory agent may be added to an ionic liquid composition for preparing the composite material in order to incorporate the anti-inflammatory agent into the composite material (e.g., after the ionic liquid is removed from the ionic liquid composition to obtain the composite material comprising the anti-inflammatory agent).

In other embodiments, the composite materials may be utilized to catalyze a reaction. For example, the composite materials may be utilized to catalyze a reaction by contacting a reaction mixture with the composite materials and optionally passing the reaction mixture through the composite material. In some embodiments, the composite material may include a reactive metal or metal cation for catalyzing the reaction (e.g., as metal or metal cation particles).

In other embodiments, the composite materials may be utilized to carry and release a compound such as a therapeutic compound (e.g., an anti-microbial compound). For example, the composite materials may be utilized to carry and release a therapeutic compound gradually over an extended period of time (e.g., a drug such as ciprofloxacin). As such, the composite material may be utilized in wound dressing material (e.g., for ulcerous infected wounds).

In other embodiments, the composite materials may be utilized to carry and release an ethylene compound (e.g., 1-methylocyclopropene). For example, the composite materials may be utilized to carry and release an ethylene compound in order to modulate ripening of fruit or freshness of flowers. As such, the composite material may be utilized in packaging material for fruit or flowers.

Accordingly, the disclosed composite materials may be configured for a variety of applications. These include, but are not limited to, filter material for use in filters for liquid or gas streams, fabric material for use in bandages for wounds, and/or packaging material for fruit or flowers.

### BRIEF DESCRIPTION OF THE FIGURES

FIG. 1. Procedure used to prepare the [CEL+CS+KER] composite materials.

FIG. 2. FTIR spectra of materials with different compositions and concentrations; Hair, wool, feather, 100% CEL, 80:20 [Wool:CEL], 80:20 [Hair:CEL] and 80:20 [Feather:CEL].

FIG. 3. X-ray diffraction spectra of (top panel): wool (dashed curve), hair (solid curve) and chicken feather (dotted curve); and (bottom panel): 80:20 wool:CEL (solid curve), 80:20 hair:CEL (dashed curve), 80:20 feather:CEL (dotted curve) and 100% CEL (line-dotted curve) composites.

FIG. 4. Surface SEM images (top two rows) and cross-sectional images (last three rows) of CEL, Wool, [Wool+CEL], [Hair:CEL] and [Feather:CEL] composites with different compositions.

FIG. 5. Plots of tensile strength as a function of % CEL in [CEL+Hair] composites (dotted curve), [CEL+Feather] composites (dashed-dotted curve) and [CEL+wool] composites (dashed curve).

FIG. 6A, FIG. 6B, FIG. 6C, and FIG. 6D. Log of reduction in number of bacteria (FIG. 6A): E. coli, (FIG. 6B): S. aureus, (FIG. 6C): MRSA, (FIG. 6D): VRE after exposure to [CEL+Hair], [CEL+Feather] and [CEL+Wool] composites for 24 hours compared to a control (no composite). Each bar represents an average of 3 measurements together with associated standard deviations.

FIG. 7. Procedure used to prepare the [CEL+KER+AgNPs] composite materials.

FIG. 8. Sample preparation for silver release from the [CEL+KER+AgNPs] composites.

FIG. 9. Schematic presentation of the FIA setup with thermal lens detection unit.

FIG. 10. FTIR spectra of [CEL+KER] composite (bottom line) and [CEL+KER+AgNPs] composite (top line).

FIG. 11. Powder X-Ray diffraction spectra of [CEL+KER+Ag+NPs] composite (top line) and [CEL+KER+AgNPs] composite (bottom).

FIG. 12A, FIG. 12B, and FIG. 12C. (FIG. 12A) SEM images of [CEL+KER+AgNPs] composite: left: surface image, right: cross section image; (FIG. 12B) EDS spectrum and (FIG. 12C) EDS images, recorded for carbon (left), silver (middle) and oxygen (right) of [CEL+KER+AgNPs] composite.

FIG. 13, FIG. 13A, FIG. 13B, FIG. 13C, FIG. 13D, and FIG. 13E. (FIG. 13) Log of growth reduction for E. coli, S. aureus, VRE, MRSA and P. aeruginosa after 24 hrs exposure to [CEL+KER+Ag+NPs] and [CEL+KER+Ag⁰NPs] composites with 3.5 mmol of silver NPs concentrations. (FIG. 13A) Log of growth reduction for E. coli after 24 hrs exposure to [CEL+KER+Ag+NPs] and [CEL+KER+Ag⁰NPs] composites with silver NPs concentrations of 3.5 mmol, 0.72 mmol and 0.48 mmol. (FIG. 13B) Log of growth reduction for S. aureus after 24 hrs exposure to [CEL+KER+Ag+NPs] and [CEL+KER+Ag⁰NPs] composites with silver NPs concentrations of 3.5 mmol, 0.72 mmol and 0.48 mmol. (FIG. 13C) Log of growth reduction for VRE after 24 hrs exposure to [CEL+KER+Ag+NPs] and [CEL+KER+Ag⁰NPs] composites with silver NPs concentrations of 3.5 mmol, 0.72 mmol and 0.48 mmol. (FIG. 13D) Log of growth reduction for MRSA after 24 hrs exposure to [CEL+KER+Ag+NPs] and [CEL+KER+AgNPs] composites with silver NPs concentrations of 3.5 mmol, 0.72 mmol and 0.48 mmol. (FIG. 13E) Log of growth reduction for P. aeruginosa after 24 hrs exposure to [CEL+KER+Ag+NPs] and [CEL+KER+Ag⁰NPs] composites with silver NPs concentrations of 3.5 mmol, 0.72 mmol and 0.48 mmol. In these figures, CEL+KER was labeled as CK; hatched bars and black bars are for (CEL+KER+Ag⁺] and [CEL+KER+Ag⁰NPS] composites, respectively. Light grey bars are for both blank ([CEL+KER] composite with no AgNPs) and control.

FIG. 14A, FIG. 14B, and FIG. 14C. Fibroblast viability based on absorbance at 490 nm after being exposed to [CEL+KER] composite, [CEL+KER+Ag⁰NPs] composite and [CEL+KER+Ag+NPs] composite for 3 days. (FIG. 14A) Composites of 15 mm in diameter were used. (FIG. 14B and FIG. 14C) Composites were of 7 mm in diameter. Each bar represents an average of 3 experiments. Error bars represent standard error of the average. (P-values are indicated as follows: (*P<0.05)). Results for control experiment (no material) are also presented. Composites causing <70% cell viability (dashed line) are considered cytotoxic.

FIG. 15A, FIG. 15B, FIG. 15C, and FIG. 15D. (FIG. 15A) Images (100×) of human fibroblasts after 3 days in the absence of any composite. (FIG. 15B) Images (100×) of human fibroblasts after 3 days with [CEL+KER] composite. (FIG. 15C) Images (100×) of human fibroblasts after 3 days with [CEL+KER] containing 0.48 mmol of Ag⁰NPs. (FIG. 15D) Images (100×) of human fibroblasts after 3 days with [CEL+KER] containing 0.72 mmol of Ag⁰NPs.

FIG. 16. Plot of concentration of silver nanoparticle released from the composites against time the composites were immersed in the solution similar to the media used in the microbial and biocompatibility assays.

FIG. 17A and FIG. 17B. (FIG. 17A) FTIR spectra of different composition of [CEL+KER] composite. (FIG. 17B) FTIR spectra of different composition of [CS+KER] composite.

FIG. 18. Deconvolution of amide band 1 into α-helix and β-sheet, woolKeratin, and woolKeratin best fit.

FIG. 19A and FIG. 19B. (FIG. 19A) Residual validation variance plot. (FIG. 19B) Explained validation variance plot.

FIG. 20. X-ray diffraction spectra of wool, regenerated KER, 25:75 CS:KER composite, and 25:75 CEL:KER composite.

FIG. 21A and FIG. 21B. (FIG. 21A) ¹³C CP-MAS NMR spectra of [CEL+KER] composite. (FIG. 21B) ¹³C CP-MAS NMR spectra of [CS+KER] composite.

FIG. 22. Surface SEM images (first and third columns) and cross-sectional images (second and fourth columns) of [CEL+KER] (first two columns on the left hand side) and [CS+KER] (last two columns on the right hand side).

FIG. 23. Plots of tensile strength as a function of % CEL in [CEL+KER] composites and % CS in [CS+KER] composites.

FIG. 24. Plots of onset decomposition temperatures for [CEL+KER] composites (open triangles) and [CS+KER] composites (filled squares).

FIG. 25. Synthesis method for [CEL+KER+Au⁰NPs] composites.

FIG. 26. FTIR spectra of [CEL+KER] composite (bottom curve) and [CEL+KER+705 μmol Au⁰NPs] composite (top curve).

FIG. 27. Powder X-ray diffractogram of [CEL+KER+705 μmol Au⁰NPs] composite.

FIG. 28A, FIG. 28B, and FIG. 28C. (FIG. 28A) SEM images of [CEL+KER+705 μmol Au⁰NPs] composite; (FIG. 28B) EDS images, recorded for gold (left), carbon (middle) and nitrogen (right) of [CEL+KER+705 μmol Au⁰NPs] composite; and (FIG. 28C) EDS spectrum of the composite.

FIG. 29. X-ray photoelectron of [CEL+KER+705 μmol Au⁰NPs] composite. (B), (C) and (D) are expanded plots of (A).

FIG. 30. Log of reduction for selected bacteria after 24 h of exposure to [CEL+KER+705 μmol Au⁰NPs]. Each bar represents an average n=3±SEM.

FIG. 31. Fibroblast viability expressed as % of control after being exposed to either no composite, to blank ([CEL+KER]), or to [CEL+KER+705 μmol Au⁰NPs], for 3 days and 7 days. Each bar represents an average of n=3±SEM. Materials causing <70% cell viability (dashed line) are considered cytotoxic.

FIG. 32. Images (100×) of human fibroblasts after 3 days (A, B, and C) and after 7 days (D, E and F): (A) and (D): in the absence of any composite; (B) and (E): with [CEL+KER] composite; and (C) and (F): with [CEL+KER+705 μmol Au⁰NPs] composite

### DETAILED DESCRIPTION

The disclosed subject matter further may be described utilizing terms as defined below.

Unless otherwise specified or indicated by context, the terms “a”, “an”, and “the” mean “one or more.” For example, “a structural polysaccharide” and “a structural protein” should be interpreted to mean “one or more structural polysaccharides” and “one or more structural proteins,” respectively.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Disclosed are composite materials and ionic liquid compositions for preparing the composite materials. The composite materials typically include one or more structural polysaccharides, one or more structural proteins, and optionally metal and/or metal oxide particles (e.g., metal microparticles and/or metal nanoparticles).

As used herein, “structural polysaccharides” refer to water insoluble polysaccharides that may form the biological structure of an organism. Typically, structurally polysaccharides are polymers of 6-carbon sugars such as glucose or modified forms of glucose (e.g., N-acetylglucosamine and glucosamine), which are linked via beta-1,4 linkages. Structural polysaccharides may include, but are not limited to cellulose, chitin, and chitosan, which may be formed from chitin by deacetylating one or more N-acetylglucosamine monomer units of chitin via treatment with an alkali solution (e.g., NaOH). Chitosan-based polysaccharide composite materials and the preparation thereof are disclosed in Tran et al., J. Biomed. Mater. Res. Part A 2013:101A:2248-2257 (hereinafter “Tran et al. 2013), which is incorporated herein by reference in its entirety.

As used herein, a “structural protein” is a protein that is used to build structural components of an organism. Suitable structural proteins for the disclosed composite materials may include fibrous structural proteins, which optionally may be referred to as “scleroproteins.” Structural proteins typically do not include globular proteins and/or membrane proteins. Structural proteins typically form long filaments which are water-insoluble. Structural proteins may comprise hydrophobic side chains that protrude from the structural protein molecule and cause structural proteins to aggregate. The peptide sequence of structural proteins typical includes a limited variety of amino acid residues and includes repeat motifs that may form secondary structures such as helices having disulfide bond between the structural protein amino acid chains. Suitable structural proteins for the disclosed composite materials may include but are not limited to one or more of keratin, collagen, elastin, and fibroin.

Suitable structural proteins may include keratin proteins. Suitable keratin proteins may include, but are not limited to, α-keratins and/or β-keratins. Keratin for use in the disclosed methods for preparing the disclosed composite materials may be derived from a number of sources, including but not limited to wool, hair (including human and non-human hair), feathers (including chicken feathers), beaks (including chicken beaks), claws (including chicken claws), and hooves of ungulates.

The disclosed composite materials may be prepared from ionic liquid compositions that comprise one or more structural polysaccharides and/or one ore more structural proteins dissolved in one or more ionic liquids. As used herein, an “ionic liquid” refers to a salt in the liquid state, typically salts whose melting point is less than about 100° C. Ionic liquids may include, but are not limited to salts based on an alkylated imidazolium cation, for example,

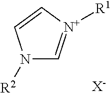

where R¹ and R² are C1-C6 alkyl (straight or branched), and X⁻ is any cation (e.g., a halide such as chloride, a phosphate, a cyanamide, or the like).

The disclosed ionic liquid compositions may be utilized in methods for preparing the disclosed composite materials that comprise a structural polysaccharide (e.g., cellulose, chitosan, chitin, and/or a mixture thereof), a structural protein (e.g., keratin), and optionally metal and/or metal oxide particles. For example, in the disclosed methods, a composite material comprising a structural polysaccharide, a structural protein, and optionally a metal and/or metal oxide particles may be prepared by: (1) obtaining or preparing one or more ionic liquid compositions as disclosed herein comprising one or more structural polysaccharides and/or one or more structural proteins, where the structural polysaccharide(s) and/or the structural protein(s) are dissolved in one or more ionic liquids to form one more ionic liquid composition(s) which optionally may be combined; optionally (2) adding a metal salt to the ionic liquid composition and optionally reducing the metal salt in situ, and (3) optionally casting the ionic liquid composition (e.g., in a mold to prepare a film or other form); (4) removing the ionic liquid from the ionic liquid composition to obtain a composite comprising the one or more structural polysaccharides, the one or more structural proteins, and the optional metal and/or metal oxide salt optionally in the form of particles.

In the disclosed methods, optionally the structural protein (e.g., keratin from wool) may first be dissolved in an ionic liquid to prepare an ionic liquid composition. Optionally, the keratin may be dissolved at a temperature at least about 80° C., 90° C., 100° C., 110° C., 120° C., 130° C., or 140° C., or within a temperature range bounded by any of these values (e.g., within a range of about 110° C.-130° C.). Preferably, the structural protein is dissolved in the ionic liquid at a temperature of at least about 120° C. Optionally, the structural protein is added to the ionic liquid a concentration of at least about 0.5%, 1%, 2%, 3%, 4%, 5%, 6%, 7%, 8%, 9%, 10%, 12%, 13%, 14%, or 15% (w/w) or no more than about 15%, 14%, 13%, 12%, 11%, 10%, 9%, 8%, 7%, 6%, 5%, 4%, 3%, 2%, 1%, 0.5%, 0.25% (w/w) or within a concentration range bounded by any of these values (e.g., 2%-10% (w/w))

In order to prepare ionic liquid compositions that include a structural protein (e.g., keratin) and a structural polysaccharide (e.g., cellulose or chitosan), preferably the structural protein is dissolved first in the ionic liquid (e.g., at a temperature within a range of about 110° C.-130° C. and preferably about 120° C.). Next, optionally the temperature of the ionic liquid composition is reduced to at least about 110° C., 100° C., 90° C., or 80° C. (e.g., about 100° C.-80° C. and preferably about 90° C.) prior to adding the structural polysaccharide (e.g., cellulose or chitosan). Optionally, the structural polysaccharide is added to the ionic liquid at a concentration of at least about 0.25%, 0.5%, 1%, 2%, 3%, 4%, 5%, 6%, 7%, 8%, 9%, 10%, 12%, 13%, 14%, or 15% (w/w) or no more than about 15%, 14%, 13%, 12%, 11%, 10%, 9%, 8%, 7%, 6%, 5%, 4%, 3%, 2%, 1%, 0.5%, 0.25% (w/w) or within a concentration range bounded by any of these values (e.g., 0.25%-0.5% (w/w)). Preferably, in order to prepare a ionic liquid composition that includes both of cellulose and chitosan, the cellulose is added to the ionic liquid first and dissolved prior to adding the chitosan to the ionic liquid and dissolving the chitosan.

The ionic liquid may be removed from the disclosed compositions by steps that include, but are not limited to washing (e.g., with an aqueous solution). The water remaining in the composite materials after washing may be removed from the composite materials by steps that include, but are not limited to drying (e.g., in air) and lyophilizing (i.e., drying under a vacuum). The composite material may be formed into any desirable shape, for example, a film or a powder (e.g., a powder of microparticles and/or particles) prior to or after removing the ionic liquid.

The disclosed composite materials may be utilized in methods for removing contaminants from aqueous solutions, liquid streams, or air streams. Chitosan-cellulose composite materials for removing microcystin are disclosed in Tran et al., J. of Hazard. Mat. 252-253 (2013) 355-366, which is incorporated herein by reference in its entirety.

The disclosed composite materials may be utilized in methods for purifying compounds from aqueous solutions, liquid streams, or air streams. In particular, the composite materials may be utilized in methods for purifying compounds from mixtures of compounds. Methods of using a chitosan-cellulose composite material for purifying a specific enantiomer of an amino acid from a racemic mixture are disclosed in Duri et al. Langmuir, 2014, 30(2), pp 642-650 (hereinafter “Duri et al. 2014”), which is incorporated herein by reference in its entirety. As disclosed in Duri et al. 2014, in methods for purifying an enantiomer of a compound from a racemic mixture of a compound, the composite material may consist of structural polysaccharides (e.g., chitosan and cellulose). As such, the presence of a metal and/or metal oxide particles within the composite material may be optional but preferred where the composite material is utilized in methods for purifying an enantiomer of a compound from a racemic mixture of a compound.

The disclosed composite materials may be utilized in methods for inhibiting or preventing growth of microbes (e.g., bacteria). For example, the disclosed composite materials may be contacted with an aqueous solution, a liquid stream, or an air stream comprising microbes to inhibit or prevent growth of microbes in the aqueous solution, the liquid stream, or the air stream. Alternatively, the disclosed composite materials may be used to coat a substrate in order to inhibit or prevent growth of microbes on the substrate. The antimicrobial properties of chitosan-based polysaccharide composite materials are disclosed in Tran et al., J. Biomed. Mater. Res. Part A 2013:101A:2248-2257 (hereinafter “Tran et al. 2013) and Harkins A L, Duri S, Kloth L C, Tran C D. 2014. “Chitosan-cellulose composite for wound dressing material. Part 2. Antimicrobial activity, blood absorption ability, and biocompatibility.” J Biomed Mater Res Part B 2014: 00B: 000-000 (hereinafter “Harkins et al. 2014”), which are incorporated herein by reference in their entireties. As disclosed in Tran et al. 2013 and Harkins et al. 2014, in methods of using the disclosed composite materials for inhibiting or preventing microbial growth, the composite material may consist of structural polysaccharides (e.g., chitosan and cellulose). The presence of metal and/or metal oxide particles within the composite material may be optional, but preferable, for example where the composite material is utilized in methods for inhibiting or preventing microbial growth.

The disclosed composite materials may include therapeutic agents. In order to prepare composite materials comprising therapeutic agents, the therapeutic agents may be added to an ionic liquid composition comprising the structural polysaccharide and structural protein dissolved therein. The present inventor has observed that the release rate for therapeutic agents incorporated in to the composite materials will vary based on the composition of the composite materials. Composite materials comprising cellulose [CEL] and chitosan [CS] or a combination of cellulose/chitosan [CEL+CS] exhibiting much faster release rates for ciprofloxacin than a composite material comprising keratin [KER]. Ciprofloxacin was released more slowly from composite materials comprising keratin and the release rate for ciprofloxacin from composite materials comprising keratin was dependent on the concentration of keratin in the composite material. Because the release rate of ciprofloxacin by [CEL+CS+KER] composites is relatively slower than a CEL composite, a CS composite, or a [CEL+CS] composite, and because the release rate is inversely proportional to the concentration of keratin in the composite, a drug such as ciprofloxacin can be encapsulated into a [CEL+CS+KER] composite, and the release of the drug from the composite can be adjusted to a selected release rate by judiciously selecting the concentration of KER in the composite.

The disclosed composite materials may include additional components such as macromolecules. In this regard, reference is made to Duri et al., “Supramolecular Composition Materials from Cellulose, Chitosan, and Cyclodextrins: Facile Preparation and Their Selective Inclusion Complex Formation with Endocrine Disruptors,” Langmuir. 2013. 29(16):5037-49, available on-line on Mar. 21, 2013; the content of which is incorporated herein by reference in its entirety. In this regard, reference also is made to Published International Application WO 2014/186702, published on Nov. 20, 2014, the content of which is incorporated herein by reference in its entirety.

Optionally, the disclosed composite materials include one or more metal and/or metal oxide particles. The disclosed metal and/or metal oxide particles may have an effective average diameter of less than about 10 μM, 5 μM, 1 μM, 0.5 μM, 0.1 μM, 0.05 μM, 0.01 or the particles may have an effective average diameter within a range bounded by any of the foregoing values as endpoints (e.g., particles having an effective average diameter within a range of 1 μM to 0.1 μM). In some embodiments, the disclosed metal and/or metal oxide particles may be referred to as “nanoparticles.”

In order to prepare composite materials comprising metal and/or metal oxide particles, the metal and/or metal oxide particles may be added to an ionic liquid composition comprising the structural polysaccharide and structural protein dissolved therein. The ionic liquid then may be removed from the composition to prepare a composite material comprising the structural polysaccharide, structural protein, and the metal or metal oxide particles. In some embodiments, a metal salt comprising a metal cation and a non-metal cation may be added to an ionic liquid composition comprising the structural polysaccharide and structural protein dissolved therein. The ionic liquid then may be removed from the composition to prepare a composite material comprising the structural polysaccharide, structural protein, and the metal salt. The metal cation of the metal salt may then be reduced in the composite in situ to create metal particles comprising elemental metal. Suitable metals and oxides thereof for the disclosed composites may include, but are not limited to, silver (Ag), gold (Au), copper (Cu), platinum (Pt), nickel (Ni), palladium (Pd), rhodium (Rh), aluminum (Al), iron (Fe), zinc (Zn), manganese (Mn), cobalt (Co), molybdenum (Mo). In some embodiments, suitable metals and oxides thereof for the disclosed composites include a transition metal.

In the synthesis method, preferably the silver nanoparticles are homogenously encapsulated and distributed in the composite during its synthesis. In the synthesis method, the nanoparticles may be recovered and recycles after each use to prevent problems associated with contamination of samples by the nanoparticles. In the synthesis method, preferably the oxidation state of silver nanoparticles (Ag⁰ or Ag⁺) can be selected by adjusting the reduction reaction. For example, the reduction reaction may be controlled to provide a composite material having a desired ratio of reduced metal versus oxidized metal (e.g., where M⁰:M⁺ is greater than about 50:50, 60:40, 70:30, 80:20, 90:10, 95:5, 96:4, 97:3, 98:2, or 99:1, or where M⁰:M⁺ is within a range bounded by any of the foregoing ratios such as a range of 70:30 to 90:10). The antimicrobial activity can be measured for composites containing different concentrations of Ag⁰ or Ag⁺.

### ILLUSTRATIVE EMBODIMENTS

The following embodiments are illustrative and should not be interpreted to limit the claimed subject matter.

### Embodiment 1

An ionic liquid composition comprising a structural polysaccharide and a structural protein dissolved in an ionic liquid.

### Embodiment 2

The composition of embodiment 1, wherein the structural polysaccharide is a polymer comprising 6-carbon monosaccharides linked via beta-1,4 linkages.

### Embodiment 3

The composition of any of the foregoing embodiments, wherein the structural polysaccharide comprises cellulose.

### Embodiment 4

The composition of any of the foregoing embodiments, wherein the structural polysaccharide comprises chitin.

### Embodiment 5

The composition of any of the foregoing, wherein the structural polysaccharide comprises chitosan.

### Embodiment 6

The composition of embodiment 5, wherein the structural protein comprises keratin.

### Embodiment 7

The composition of any of the foregoing embodiments, further comprising metal nanoparticles and/or metal oxide nanoparticles.

### Embodiment 8

The composition of embodiment 7, wherein the metal nanoparticles comprise gold, silver, or copper nanoparticles and/or wherein the metal oxide nanoparticles comprise gold, silver, or copper oxide nanoparticles.

### Embodiment 9

The composition of any of the foregoing embodiments, wherein the ionic liquid is an alkylated imidazolium salt.

### Embodiment 10

The composition of embodiment 9, wherein the alkylated imidazolium salt is selected from a group consisting of 1-butyl-3-methylimidazolium salt, 1-ethyl-3-methylimidazolium salt, and 1-allyl-3-methylimidazolium salt.

### Embodiment 11

The composition of any of the foregoing embodiments, wherein the ionic liquid is 1-butyl-3-methylimidazolium chloride.

### Embodiment 12

The composition of any of the foregoing embodiments, wherein the ionic liquid composition comprises at least 4% w/w of the dissolved structural polysaccharide.

### Embodiment 13

The composition of any of the foregoing embodiments, wherein the ionic liquid composition comprises at least 10% w/w of the dissolved structural polysaccharide.

### Embodiment 14

A method for preparing a composite material comprising one or more structural polysaccharides, one or more structural polypeptides, and optionally metal nanoparticles and/or metal oxide nanoparticles, the method comprising preparing a ionic liquid composition according to any of the foregoing embodiments and removing the ionic liquid to retain the composite material.

### Embodiment 15

The method of embodiment 14, wherein the composite material comprises metal oxide nanoparticles and the method further comprises contacting the metal oxide nanoparticles with a reducing agent.

### Embodiment 16

The method of embodiment 15, wherein the reducing agent comprises watermelon rind.

### Embodiment 17

The method of any of embodiments 14-16, wherein the ionic liquid is removed by steps that include washing the ionic liquid composition with an aqueous solution to obtain the composite material and drying the composite material thus obtained.

### Embodiment 18

A composite material prepared by the method of any of embodiments 14-17.

### Embodiment 19

A method for removing a contaminant from a stream, the method comprising contacting the stream and the composite material of embodiment 18.

### Embodiment 20

A method for killing or eliminating microbes, the method comprising contacting the microbes with the composite material of embodiment 18.

### Embodiment 21

A method of purifying a compound from a stream, the method comprising contacting the compound with the composite material of embodiment 18.

### Embodiment 22

The method of embodiment 21, wherein the compound is an enantiomer and the stream comprises a racemic mixture of the compound.

### Embodiment 23

A method for catalyzing a reaction, the method comprising contacting a reaction mixture with the composite material of embodiment 18.

### Embodiment 24

A method for delivering a compound, the method comprising contacting the compound with the composite material of embodiment 18 and allowing the compound to diffuse from the composite material.

### Embodiment 25

A filter comprising the composite material of embodiment 18.

### Embodiment 26

A bandage comprising the composite material of embodiment 19.

### Embodiment 27

A method of purifying an enantiomer of a compound from a racemic mixture of the compound, the method comprising contacting the racemic mixture with a composite material, wherein the composite material is prepared by dissolving a structural polysaccharide and a structural protein in an ionic liquid to form an ionic liquid composition, optionally adding metal nanoparticles or metal oxide nanoparticles to the ionic liquid composition, and thereafter removing the ionic liquid from the ionic liquid composition to obtain the composite material.

### Embodiment 28

The method of embodiment 27, wherein the structural polysaccharide is a mixture of cellulose and chitosan.

### Embodiment 29

The method of embodiment 27 or 28, wherein the structural protein is keratin.

### Embodiment 30

The method of any of embodiments 27-29, wherein the metal nanoparticles comprise gold, silver, or copper nanoparticles, and/or the metal oxide nanoparticles comprise gold-, silver- or copper oxide nanoparticles.

### EXAMPLES

The following examples are illustrative and are not intended to limit the claimed subject matter.

### Example 1—Synthesis, Structure and Antimicrobial Property of Green Composites from Cellulose, Wool, Hair and Chicken Feather

Reference is made to Tran et al., “Synthesis, structure and antimicrobial property of green composites from cellulose, wool, hair and chicken feather,” Carbohydrate Polymers, 151 (2016) 1269-1276, the content of which is incorporated herein by reference in its entirety.

Abstract

Novel composites between cellulose (CEL) and keratin (KER) from three different sources (wool, hair and chicken feather) were successfully synthesized in a simple one-step process in which butylmethylimidazolium chloride (BMIm⁺Cl⁻), an ionic liquid, was used as the sole solvent. The method is green and recyclable because [BMIm⁺Cl⁻] used was recovered for reuse. Spectroscopy (FTIR, XRD) and imaging (SEM) results confirm that CEL and KER remain chemically intact and homogeneously distributed in the composites. KER retains some of its secondary structure in the composites. Interestingly, the minor differences in the structure of KER in wool, hair and feather produced pronounced differences in the conformation of their corresponding composites with wool has the highest α-helix content and feather has the lowest content. These results correlate well with mechanical and antimicrobial properties of the composites. Specifically, adding CEL into KER substantially improves mechanical strength of [CEL+KER] composites made from all three different sources, wool, hair and chicken feathers (i.e., [CEL+wool], [CEL+hair] and [CEL+feather]. Since mechanical strength is due to CEL, and CEL has only random structure, [CEL+feather] has, expectedly, the strongest mechanical property because feather has the lowest content of α-helix. Conversely, [CEL+wool] composite has the weakest mechanical strength because wool has the highest α-helix content. All three composites exhibit antibacterial activity against methicillin resistant S. aureus (MRSA). The antibacterial property is due not to CEL but to the protein and strongly depends on the type of the keratin, namely, the bactericidal effect is strongest for feather and weakest for wool. These results together with our previous finding that [CEL+KER] composites can control release of drug such as ciprofloxacin clearly indicate that these composites can potentially be used as wound dressing.

Introduction

Sustainability, industrial ecology, eco-efficiency, and green chemistry are directing the development of the next generation of materials. Biodegradable and biocompatible materials generated from renewable biomass feedstock are regarded as promising materials that could replace synthetic polymers and reduce global dependence on fossil fuel sources. The most abundant biorenewable biopolymers on the earth include polysaccharide such as cellulose and keratin (wool, hair and chicken feather).

Keratins (KER) are a group of cysteine-rich fibrous proteins found such materials as wools, hairs, chicken feather, nails (Dullaart, R. & Mousquès, J., 2012). Of particular interest are hairs and chicken feathers as these materials are an important waste product from the salons and poultry industry but are generally left untreated because they have limited solubility and cannot be easily and economically converted to environmentally benign products (Verma et al., 2008; Vilaplana et al., 2010). Keratins are known to possess advantages for wound care, tissue reconstruction, cell seeding and diffusion, and drug delivery as topical or implantable biomaterial (Cui et al., 2013; Hill et al., 2010; Justin et al. 2011; Vasconcelos et al., 2013). As implantable film, sheet, or scaffold, keratins can be absorbed by surrounding tissue to provide structural integrity within the body while maintaining stability under mechanical load, and in time can break down to leave neo-tissue (Cui et al., 2013; Hill et al., 2010; Justin et al. 2011; Vasconcelos et al., 2013; Verma et al., 2008). The abundance and regeneration nature of wools, hairs and feathers coupled with the ability to be readily to be converted into biomaterials have made KER a subject of intense study (Justin et al. 2011; Vasconcelos et al., 2013; Vilaplana et al., 2010).

Unfortunately, KER has relatively poor mechanical properties, and as a consequence, materials made from KER lack the stability required for medical applications (Cui et al., 2013; Hill et al., 2010; Sando et al., 2010; Vasconcelos et al., 2013; Verma et al., 2008). To increase the structural strength of KER-based materials, attempts have been made to cross-link KER chains with a crosslinking agent or convert functional groups on its amino acid residues via chemical reaction(s) (Justin et al. 2011; Sando et al., 2010; Vasconcelos et al., 2013). The rather complicated, costly and multistep process is not desirable as it may inadvertently alter its unique properties, making the KER-based materials less biocompatible and toxic, and removing or lessening its unique properties. A new method which can improve the structural strength of KER-based products not by chemical modification with synthetic chemicals and/or synthetic polymers but rather by use of naturally occurring polysaccharides such as CEL, is particularly needed.

We have demonstrated recently that a simple ionic liquid, butylmethylimmidazolium chloride ([BMIm⁺Cl⁻]), can dissolve polysaccharides such as CEL and chitosan (CS), and by use of this [BMIm⁺Cl⁻] as the sole solvent, we developed a simple, green and totally recyclable method to synthesize [CEL+CS] composites just by dissolution without using any chemical modifications or reactions (Duri & Tran, 2013; Harkins et al., 2014; Mututuvari & Tran, 2013; Mututuvari & Tran, 2014; Tran et al., 2013a; Tran et al, 2013b). The [CEL+CS] composite obtained was found to be not only biodegradable and biocompatible but also retain unique properties of its components. Since [BMIm⁺Cl⁻] can also dissolve wool keratin (Chen et al, 2014; Xie et al, 2005), it may be possible to use this IL as a solvent to synthesize composites containing CEL and keratin. In fact, Xie et al have shown that wool keratin can be regenerated by initially dissolving in [BMIm⁺Cl⁻] and subsequently precipitated from methanol, and with this procedure, there were able to synthesize a 1/5 wool keratin/cellulose composite (Xie et al, 2005). Recently, by using [BMIm⁺Cl⁻] as a sole solvent we were able to synthesize composites from cellulose, chitosan and wool keratin with different compositions and concentrations (Tran & Mututuvari). More importantly, we demonstrated that the composites can be used for drug delivery as the kinetics of the release can be controlled by adjusting the concentration of wool keratin in the composite (Mututuvari & Tran, 2014).

Such consideration prompted us to initiate this study which aims to improve the mechanical properties of the KER-based composites by adding CEL to the composites, and to demonstrate that the composites will retain unique properties of their components. Since KER is known to have different structure and conformation depending on the source, (i.e., wool, hair or chicken feather) we synthesized [CEL+KER] composites with KER from either of wool, hair or chicken feather. Various spectroscopic and imaging techniques including FTIR, powder X-ray diffraction, SEM and tensile strength were employed to characterize the composites and to determine their structure and property. Microbial assays were carried out to determine antimicrobial property of the composites, results obtained were correlated with the structure and conformation of the composites to formulate structure-property relationship for the composites. The results of our initial investigation are reported herein.

Methods

Chemicals.

Microcrystalline cellulose (DP≈300) was purchased from Sigma-Aldrich (Milwaukee, Wis.). Untreated hair from local saloons and chicken feathers from local poultry farms were washed with 0.5% SDS aqueous solution, rinsed with fresh water and air-dried, followed with additional cleaning by Soxhlet extraction with petroleum ether for 48 hrs. Raw sheep wool (untreated), obtained from a local farm, was cleaned by Soxhlet extraction with a 1:1 acetone/ethanol mixture for 48 hrs. [BMIm⁺Cl⁻] was prepared from freshly distilled 1-methylimidazole and n-chlorobutane (both from Alfa Aesar, Ward Hill, Mass.) using method previously reported (Duri & Tran, 2013; Haverhals et al., 2012).

Instruments.

FTIR spectra (from 450-4,000 cm⁻¹ were recorded on a Spectrum 100 Series FTIR spectrometer (Perkin Elmer, USA) at resolution of 2 cm⁻¹ by the KBr method. Each spectrum was an average of 64 individual spectra. X-ray diffraction (XRD) measurements were taken on a Rigaku MiniFlex II diffractometer utilizing the Ni filtered Cu Kα radiation (1.54059 Å). The voltage and current of the X-ray tube were 30 kV and 15 mA respectively. The samples were measured within the 20 angle range from 2.0 to 85. The scan rate was 50 per minute. Data processing procedures were performed with the Jade 8 program package (Duri et al., 2010). The surface and cross-sectional morphologies of the composite films were examined under vacuum with a JEOL JSM-6510LV/LGS Scanning Electron Microscope with standard secondary electron (SEI) and backscatter electron (BEI) detectors. Prior to SEM measurement, the film specimens were made conductive by applying a 20 nm gold-palladium-coating onto their surfaces using an Emitech K575x Peltier Cooled Sputter Coater (Emitech Products, TX). The tensile strength of the composite films were evaluated on an Instron 5500R tensile tester (Instron Corp., Canton, Mass.) equipped with a 1.0 kN load cell and operated at a crosshead speed of 5 mm min⁻¹. Each specimen had a gauge length and width of 25 mm and 10 mm respectively. Thermogravimetric analyses (TGA) (TG 209 F1, Netzsch) of the composite films were investigated at a heating rate of 10° C. min⁻¹ from 30-600° C. under a continuous flow of 20 mL min⁻¹ nitrogen gas.

In Vitro Antibacterial Assays.

Nutrient broth (NB) and nutrient agar (NA) were obtained from VWR (Radnor, Pa.). The bacterial cultures used in this study were obtained from the American Type Culture Collection (ATCC, Rockville, Md.). Seven different composites with different compositions and concentrations were used. They were 40:60 Hair:CEL; 40:60 Feather:CEL, 65:35 Hair:CEL, 65:35 Feather:CEL, 80:20 Hair:CEL, 75:25 Feather:CEL and 90:10 Hair:CEL.

The composites were tested for antibacterial activity on model bacterial strains E. coli (ATCC 8739), Staphylococcus aureus (ATCC 25923), methicillin resistant S. aureus (ATCC 33591), vancomycin resistant Enterococcus faecalis (ATCC 51299), and Pseudomonas aeruginosa (ATCC 9027) using previously published protocol (Harkins et al., 2014; Mututuvari et al., 2013; Tran et al., 2013a).

Preparation of the overnight bacterial culture included inoculation of 10 mL of nutrient broth medium with a culture that was maintained on a blood agar at 4° C. using an inoculation loop. The culture was then incubated overnight at 37° C. and 150 rpm. The next day the composites were placed in the sterile tubes with 2 mL of nutrient broth, which was then inoculated with 2 μL of the overnight culture. The tubes were then sampled at time 0 and placed into an incubator at 37° C. and 600 rpm for 24-hour incubation. The samples taken at time 0 were then diluted to desirable dilutions, plated onto nutrient agar, and incubated overnight at 37° C. The next day the colony forming units (CFUs) were counted on statistically significant plates: 30-300 (CFUs) using the standard plate counts, also known as plate count agar (PCA) method (Jorgensen et al. 2009). The tubes were again sampled at time 24 hours and the dilution and plating procedure from the previous day was repeated. The plates were incubated overnight at 37° C. The next day the CFUs were counted again. From the CFU data obtained from time 0 and 24 hours, log of reduction of bacteria defined as follows was calculated for each experiment:

$$Log of reduction = log \frac{{N}_{0}}{{N}_{t}}$$

where N₀ is the number of bacteria at the beginning of the experiment, and Nₜ is the number of bacteria after 24 hours.

Results and Discussion

Fourier Transform Infrared (FTIR).

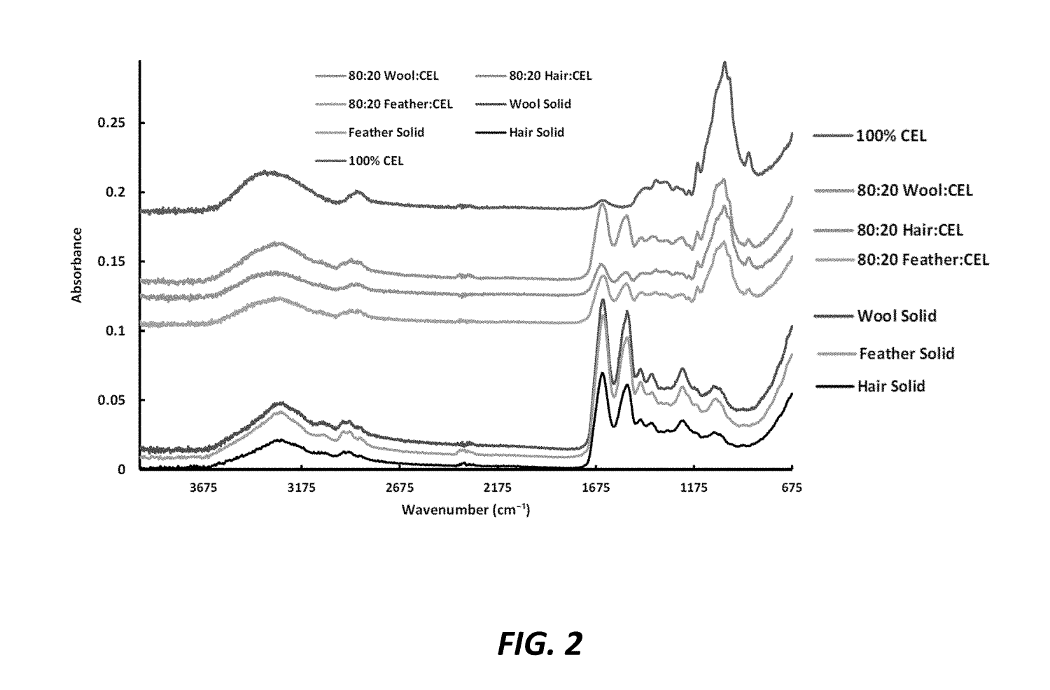

FTIR was used to confirm that ionic liquid does not produce any chemical alterations during the dissolution of wool, hair, chicken feather, and CEL and the synthesis the [Wool+CEL], [Hair+CEL] and [Feather+CEL] composites, and to characterize the composites. Shown in FIG. 2 are the FT-IR spectra of the CEL powder, wool, hair and chicken feather as well as of the composites (80:20 wool:CEL, 80;20 hair:CEL and 80:20 feather:CEL). The spectra of the starting materials, wool, hair and feather are very similar which is as expected as these materials contain keratin, and the only difference among them is a few amino acid residues and some differences in their secondary structures. All three materials exhibit several bands including two large bands at around 1520 cm⁻¹ and 1643 cm⁻¹ (bending of the N—H of the amide bands), and the 1216 cm⁻¹ band which can be attributed to the in phase combination of the N—H bending and the C—N stretch vibrations (amide III) (Greve et al., 2008; Sowa et al., 1995). It is noteworthy to add that the FTIR spectrum of wool does not have any band at 1745 cm⁻¹, which is known to be due to lipid ester carbonyl vibrations (Tanabe et al., 2002). It seems, therefore, that the Soxhlet extraction effectively removed all residual lipids from wool. For reference, the spectrum of CEL powder was also taken. It exhibits several distinct different bands at around 1350 cm⁻¹, 1147 cm⁻¹ and 800 cm⁻¹ which can be tentatively, assigned to the O—H bending vibration, the C—O stretching (of the C—OH group) and the C—H stretching, respectively (Duri & Tran, 2013; Harkins et al., 2014; Mututuvari & Tran, 2014; Tran et al., 2013a; Tran et al, 2013b).

The spectra of composites between 20% CEL and 80% of either of wool, hair or feather are also presented in FIG. 2. As expected, the spectra of these composites exhibit bands characteristic of their respective components, namely, the bands at 1520 cm⁻¹, 1643 cm⁻¹ and 1216 cm⁻¹ from KER and the 1350 cm⁻¹, 1147 cm⁻¹ and 800 cm⁻¹ bands of CEL. Furthermore, the magnitude of these bands seems to correlate well with the concentration of corresponding component in the film. For example; the bands due to CEL in the composites correspond to 20% to those in the CEL powder whereas the KER bands are about 80% to those of wool, hair and feather.

Powder X-Ray Diffraction (XRD).

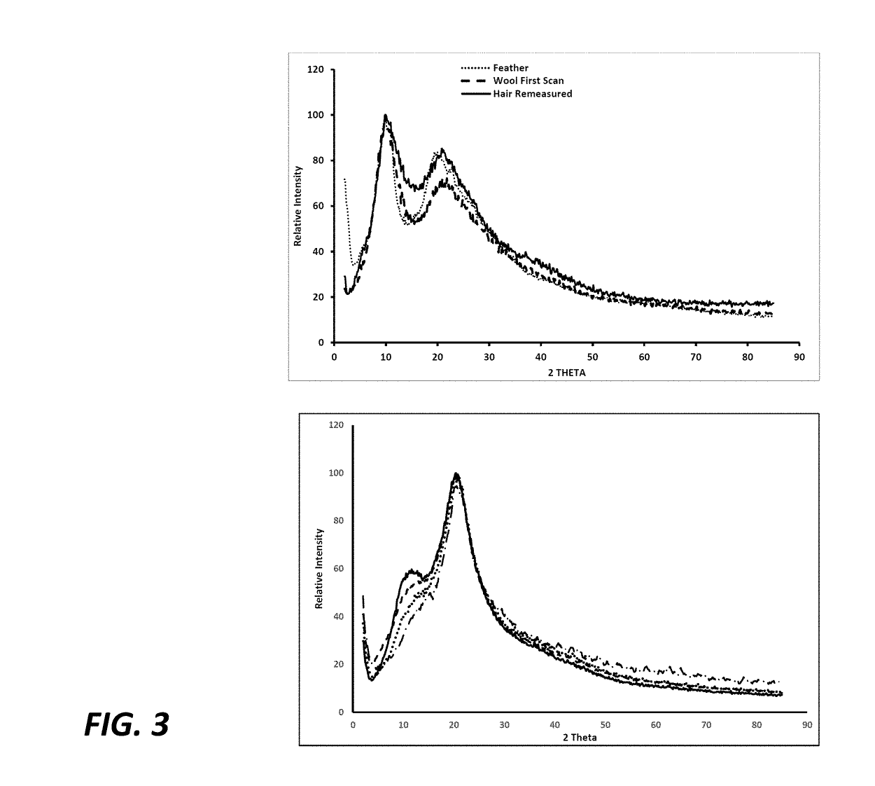

FIG. 3 (top panel) shows XRD spectra for wool, hair and chicken. Wool (dashed curve) exhibits two bands at 2θ of about 9° and 20°. They can be attributed to the α-helix and other structures including β-sheet and random form, respectively (Appelbaum et al., 2007; McKittrick et al., 2012). As expected, hair (solid curve) and feather (dotted curve) also have similar spectrum as that of wool. However, the relative intensity of the two bands at 9° and 20° for hair and feather are different from that of wool. Since the total intensity, or rather the area under these two bands are the same (i.e., 100% or total structure of the composite which includes α-helix and other structures including β-sheet and random form), the fact that the bands at 2θ=20° for both hair and feather are of relatively higher intensity than that of wool while their α-helix bands at 9° are similar to that of wool clearly indicates that the α-helix content is highest for wool followed by hair with feather has the lowest content.

XRD spectra of 80:20 wool:CEL (dashed curve), 80:20 hair:CEL (solid curve), 80:20 feather:CEL (dotted curve) and 100% CEL (line-dotted curve) composites are also presented in FIG. 3 (bottom panel). Different from pure wool, hair and feather, all three composites exhibit a pronounced band at around 2θ=20° and a shoulder at 2θ=9°. In fact the spectra of all three composites are similar to the spectrum of the regenerated 100% CEL which is known to have only random structure. These results seem to indicate that adding CEL to these KER materials substantially decreases the α-helix structure while increase the β-sheet and other forms. It seems that during the dissolution with [BMIm⁺Cl⁻], the inter- and intra-molecular bonds in wool, hair and feather were broken thereby destroying its secondary structure while maintaining its primary structure. During gelation, regeneration from water and drying, these interactions were reestablished thereby partially reforming some of the original secondary structure. However, in the presence of CEL the chains are maintained in the extended form thereby hindering a significant reformation of the α-helix. Consequently, the composites formed may adopt structures with relatively lower content of α-helix and higher β-sheet content.

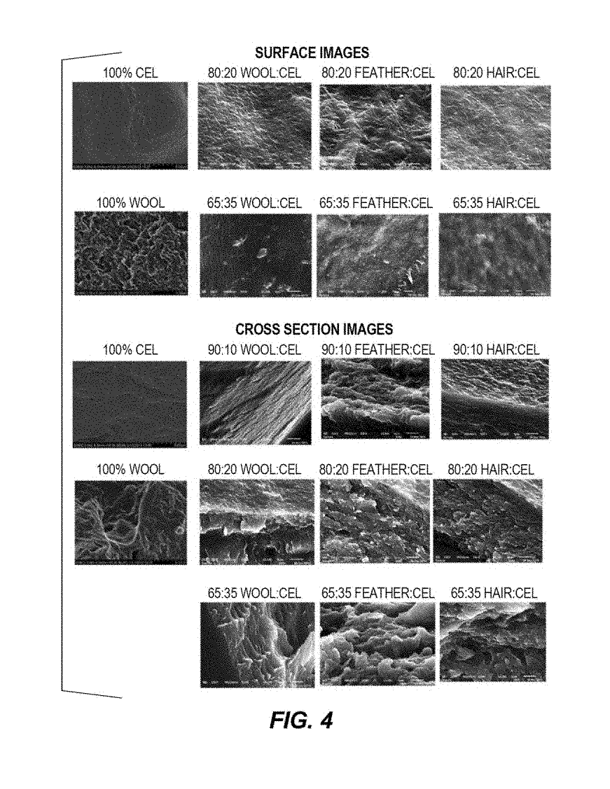

Scanning electron microscope (SEM). FIG. 4 shows SEM images of the surfaces and cross sections of regenerated 100% CEL, 100% wool, [CEL+Wool], [CEL+Hair] and [CEL+Feather] composites with different compositions. While images for 100% CEL exhibit smooth and homogeneous morphologies without any pores, the images of 100% wool exhibit a rough and porous structure with a three dimensional interconnection throughout the film surface. This porous structure seems to reflect the physical properties of KER films, namely the brittleness of the regenerated 100% wool film, and the fact that it was not possible for us to regenerate 100% hair and 100% feather films as they were found to be too brittle. CEL was added to wool, hair and feather to improve mechanical property of the composites. From both surface and cross sections SEM images of [wool+CEL], [feather+CEL] and [hair+CEL] at various compositions (90:10, 80:20 and 65:35) it is clear that CEL forms homogenous composites with all three proteins and at all compositions. As expected, adding KER to the proteins introduces roughness to the composites. Moreover, the microstructures of the composites are dependent on the source of KER (i.e., wool, hair or feather) are noticeably different from one another. For example, 90:10 wool:CEL composite seems to be somewhat rougher than 100% CEL and 100% wool. It is, however, relatively finer than the corresponding 90:10 hair:CEL composite. On the other hand, the 90:10 feather:CEL composite exhibits highest degree of roughness. Again these results seem to correlate with results presented above on the conformation of the proteins, namely, because wool has the highest α-helix content, when mix with CEL, it still can retain some of its structure, thereby producing composites with relatively finer structure than those of hair and feather. Conversely, feather which has the lowest α-helix content, does not seem to be able to mix well with CEL. As a consequence, the resultant composites have the highest degree of roughness compared to corresponding wool and hair composites. Since CEL has distinctly different structure from wool, hair and feather, increasing concentration of CEL in the composite from 10% to 20% and 35% leads to increase in the roughness of the composites. Again, as expected, for the same composition, the roughness is highest for the feather:CEL composite followed by hair:CEL composite with the wool:CEL composite has the lowest roughness structure.

Mechanical Properties.

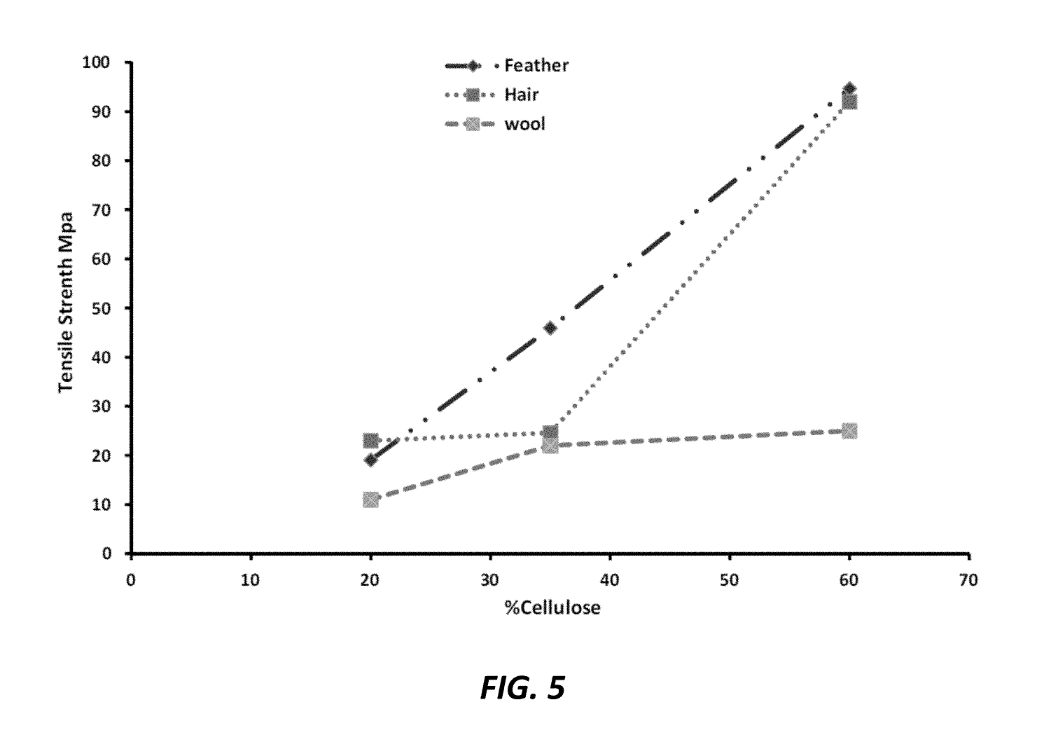

It is known that KER can encapsulate and control release of drugs.²⁶ However, its poor mechanical properties continue to hamper its potential applications. For example, as previously reported and also observed in this study, regenerated KER film was found to be too brittle to be reasonably used in any application (Hill et al., 2010; Sando et al., 2010; Vasconcelos et al., 2013; Verma et al., 2008). Since CEL is known to possess superior mechanical strength, it is possible enhance the mechanical property of KER-based composite by adding CEL into it. Accordingly, CEL was added to either wool, hair or feather to prepare [Wool+CEL], [Hair+CEL] and [Feather+CEL] composites with different concentrations. In FIG. 5, the tensile strength of the composites was plotted as a function of cellulose content. As expected, adding CEL to either wool, hair or feather substantially increases the tensile strength of the composites. For example, the tensile strength of 80:20 Feather:CEL composite (dashed-dotted curve) increased from 19.08 MPa to 45.93 MPs or ˜2.5× when CEL loading was increased from 20% to 35%. Up to a 5× increase was observed when CEL loading was increased to 60% (i.e., 94.66 MPa). The same effect was also observed for [Wool+CEL] composites (dashed curve) and [Hair+CEL] composites (dotted curve) as well. Interestingly, enhancement effect induced by CEL is highest for [Feather+CEL] composites and lowest for [Wool+CEL] composites. This may be due to the effect CEL has on the secondary structure of KER in feather, hair and wool. As described in previous section, X-ray diffraction results indicate that for the same CEL loading, the α-helix content is highest for [wool+CEL] composites followed by [Hair+CEL] composites with [Feather+CEL] composites have the lowest content. That is, the interactions between CEL and feather are strongest whereas the weakest is between CEL and wool. KER can, therefore retain relatively less secondary structure or less α-helix content in the [Feather+CEL] composites compared to [Wool+CEL] and [Hair+CEL] composites. Since CEL can interact stronger with feather, it would impart more mechanical strength to feather than to wool or hair. Consequently, [Feather+CEL] composites have stronger mechanical strength than [Hair+CEL], and [Wool+CEL] composites have the weakest mechanical strength.

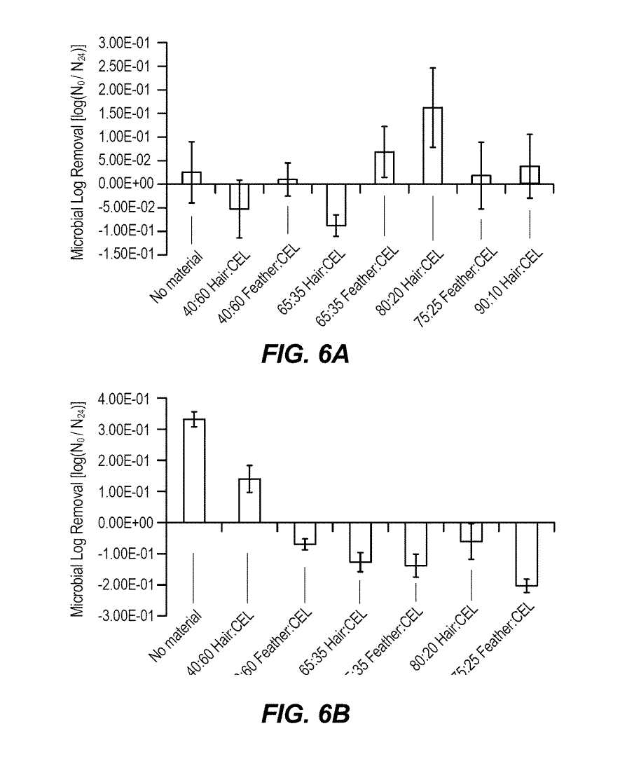

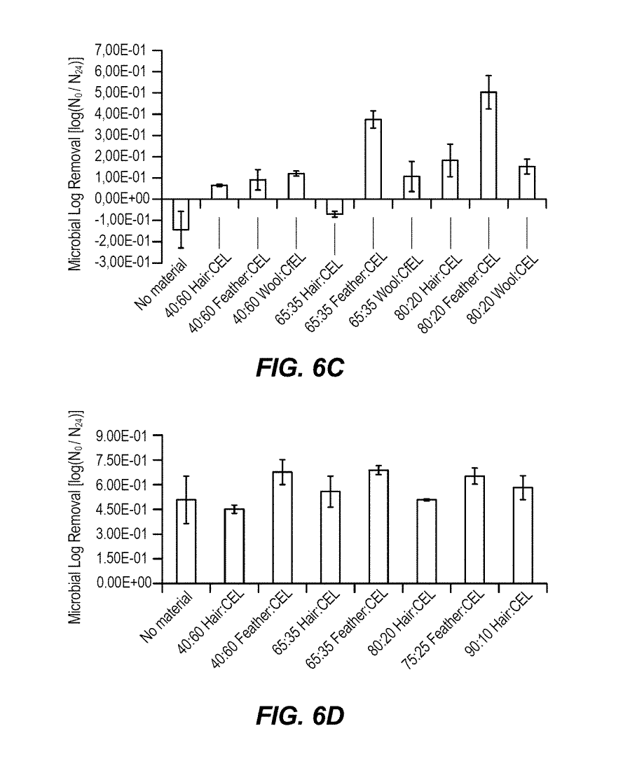

Antibacterial assays. Experiments were then to carry out to determine the composites have any effect on selected gram negative (E. coli, P. aeruginosa) and gram positive bacteria (S. aureus, MRSA, VRE). Different types of composites ([Hair+CEL], [Feather+CEL] and [Wool+CEL]) with different concentrations (40:60, 65:35, 75:25 and 80:20 of either wool, hair or feather and CEL) were evaluated by growing the bacteria in the presence of the composites for 24 hours and then plated out onto nutrient agar plates. The number of colonies formed after overnight incubation was compared to a standard growth control. Results obtained, plotted as Microbial Log Removal are shown in FIG. 6A-D for E. coli, S. aureus, MRSA and VRE. It is evident from FIGS. 6A, B and D, that within experimental errors, all three composites ([CEL+Hair], [CEL+Feather] and [CEL+wool]) did not inhibit any observable antimicrobial activity against E. coli, S. aureus and VRE. Interestingly, all three composites did show some antibacterial activity against MRSA, and the antimicrobial activity is dependent not only the on the type of the protein but also on its relative concentration as well. For examples, the 65:35 Wool:CEL exhibited very small if any effect whereas the 65:35 Feather:CEL did show substantially strong antimicrobial effect against MRSA. Hair:CEL composites seem to have relatively stronger effect than wool but weaker than feather, namely, at 80% protein content, the [Hair:CEL] exhibit somewhat stronger than that by 80:20 Wool:CEL but still much weaker than that of 80:20 Feather:CEL. Together, the results seem to indicate that similar to our previous work on the [CEL+chitosan] composites, CEL does not have any antimicrobial activity at all (Harkins et al., 2014; Tran et al., 2013a). The antibacterial property is due only to protein but also to the specific type of the keratin as well. That is, the bactericidal effect is strongest for feather followed by hair and the weakest is for wool. Taken together the antimicrobial effect and the secondary structure results presented in the previous section, suggest that feather with its highest content of random structure (i.e., lowest α-helix content) can readily interact with MRSA which enable it to exhibit strongest antimicrobial activity. Conversely, wool with its highest α-helix content, has relatively more defined structure which somewhat restricts its ability to interact with bacteria. As a consequence, it has the lowest antimicrobial activity. Hair with its structure in the middle of feather and wool, has the middle range of antimicrobial effect.

### Discussion

In summary, we have shown that composites between CEL and keratin from three different sources (wool, hair and feather) were successfully and readily synthesized in a simple one-step process in which [BMIm⁺Cl⁻], an ionic liquid, was used as the sole solvent. The method is green and recyclable because majority of [BMIm⁺Cl⁻] used was recovered for reuse. Results of spectroscopy (FTIR, XRD) and imaging (SEM) measurements confirm that CEL and KER (from all three sources: wool, hair and chicken feather) remain chemically intact and homogeneously distributed in the composites. KER also retains some of its secondary structure in the composites. Interestingly, the minor differences in the compositions of KER in wool, hair and feather magnifies into pronounced differences in the structure of wool, hair and feather and their corresponding composites with wool has the highest content of α-helix, followed by hair and feather has the lowest content. These results correlate well with SEM results and properties (mechanical and antimicrobial properties) of the composites. Specifically, adding CEL into KER substantially improves mechanical strength of all three composites ([CEL+wool], [CEL+hair] and [CEL+feather]. Since mechanical strength is due to CEL, and CEL has only random structure, [CEL+feather] has, expectedly, the strongest mechanical property because feather has the lowest content of α-helix. Conversely, [CEL+wool] composite has the weakest mechanical strength because wool has the highest α-helix content. All three composites, [Feather+CEL], [Hair+CEL] and [Wool+CEL] were found to exhibit antibacterial activity against MRSA. The antibacterial property is due not to CEL but rather to the protein and is strongly dependent on the type of the keratin. That is, the bactericidal effect is strongest for feather followed by hair and the weakest is for wool. For example, up to 1.5 log and 1.75 logs of reduction of MRSA growth were observed in the presence of 80:20 Wool:CEL and Hair:CEL composites, respectively. Remarkably, the Feather:CEL composite with the same composition exhibits up to 5 log of reduction for growth of MRSA. These results together with our previous finding that [CEL+KER] composites can be used for drug delivery as the kinetics of the release can be controlled by adjusting the concentration of wool keratin in the composite (Mututuvari & Tran, 2014), clearly indicate that the composites can be used as dressing to treat ulcerous wounds. Moreover, the research reported here also has profound beneficial effect on the environment as it provide a facile, green and recyclable method to readily convert otherwise polluted substances such as wool (waste product from textile industry), hair and chicken feather into biocompatible and useful materials for water purification and wound healing.

### REFERENCES

(1) Appelbaum, P. C. (2007). Microbiology of antibiotic resistance in Staphylococcus aureus. Clinical Infectious Diseases, 45(Suppl. 3), S 165-S 170.
(2) Chen, J., Vongsanga, K., Wang, X., & Byrne, N. (2014). What happens during natural protein fibre dissolution in ionic liquids. Material, 7, 6158-6168.
(3) Cilurzo, C., Selmin, F., Aluigi, A., & Bellosta, S. (2013). Regenerated keratin proteins as potential biomaterial for drug delivery. Polymers for Advance Technologies, 24, 1025-1028.
(4) Cui, L., Gong, J., Fan, X., Wang, P., Wang, Q., & Qiu, Y. (2013). Trans glutaminase-modified wool keratin film and its potential application in tissue engineering. Engineering in Life Sciences, 13, 149-155.
(5) Dullaart, R., & Mousquès, J. (Eds.). (2012). Keratin: structure, properties, and applications. In. Hauppauge, N.Y: Nova Science Publishers.
(6) Duri, S., & Tran, C. D. (2013). Supramolecular composite materials from cellulose, chitosan and cyclodextrin: facile preparation and their selective inclusion complex formation with endocrine disruptors. Langmuir, 29, 5037-5049.
(7) Duri, S., Majoni, S., Hossenlopp, J. M., & Tran, C. D. (2010). Determination of chemical homogeneity of fire retardant polymeric nanocomposite materials by near-infrared multispectral imaging microscopy. Analytical Letters, 43, 1780-1789.
(8) Greve, T. M., Andersen, K. B., & Nielsen, O. F. (2008). Penetration mechanism of dimethyl sulfoxide in human and pig ear skin: an ATR-FTIR and near-FT Raman Spectroscopic in vivo and in vitro study. Spectroscopy, 22, 405-417.
(9) Harkins, A. L., Duri, S., Kloth, L. C., & Tran, C. D. (2014). Chitosan-cellulose composite for wound dressing material. Part 2. Antimicrobial activity, blood absorption ability, and biocompatibility. Journal of Biomedical Materials Research Part B, 102, 1199-1206.
(10) Haverhals, L. M., Reichert, W. M., Nazare, N., Zammarano, M., Gilman, J. W., De Long, H. C., et al. (2012). Ionic liquid facilitated introduction of functional materials into biopolymer polymer substrates, in molten salts and ionic liquids 18. ECS Transactions, Vol. 50(11), 631-640.
(11) Hill, P., Brantley, H., & Van Dyke, M. (2010). Some properties of keratin biomaterials: kerateines. Biomaterials, 1, 585-593.
(12) Jorgensen, J. H., Ferraro, M. J., Jorgensen, J. H., & Ferraro, M. J. (2009). Antimicrobial susceptibility testing: a review of general principles and contemporary practices. Clinical Infectious Diseases, 49(11), 1749-1755.
(13) Justin, M., Saul, M. D., Ellenburg, R., de Guzman, C., & Van Dyke, M. (2011). Keratin hydrogels support the sustained release of bioactive ciprofloxacin. Journal of Biomedical Materials Research Part A, 98(A), 544-553.
(14) McKittrick, J., Chen, P. Y., Bodde, S. G., Yang, W., Novitskaya, E. E., & Meyers, M. A. (2012). The structure, functions, and mechanical properties of keratin. JOM, 64, 449-468.
(15) Mututuvari, T. M., & Tran, C. D. (2014). Synergistic adsorption of heavy metal ions and organic pollutants by polysaccharide supramolecular composite materials from cellulose, chitosan and crown ether. Journal of Hazardous Materials, 264, 449-459.
(16) Mututuvari, T. M., Harkins, A. L., & Tran, C. D. (2013). Facile synthesis, characterization and antimicrobial activity of cellulose-Chitosan-Hydroxy apatite composite material, a potential material for bone tissue engineering. Journal of Biomedical Materials Research Part A, 101(11), 3266-3277.
(17) Sando, L., Kim, M., Colgrave, M. L., Ramshaw, J. A., Werkmeister, J. A., & Elvin, C. M. (2010). Photochemical crosslinking of soluble wool keratins produces a mechanically stable biomaterial that supports cell adhesion and proliferation. Journal of Biomedical Materials Research Part A, 95, 901-911.
(18) Sowa, M. G., Wang, J., Schultz, C. P., Ahmed, M. K., & Mantsch, H. H. (1995). Infrared spectroscopic investigation of in vivo and ex vivo human nails. Vibrational Spectroscopy, 10, 49-56.
(19) Tanabe, T., Okitsu, N., Tachibana, A., & Yamauchi, K. (2002). Preparation and characterization of keratin-chitosan composite film. Biomaterials, 23, 817-825.
(20) Tran, C. D., & Mututuvari, T. M. (2015). Cellulose, chitosan and keratin composite materials controlled drug release. Langmuir, 31, 1516-1526.
(21) Tran, C. D., Duri, S., & Harkins, A. L. (2013). Recyclable synthesis, characterization, and antimicrobial activity of chitosan-based polysaccharide composite materials. Journal of Biomedical Materials Research Part A, 101, 2248-2257.
(22) Tran, C. D., Duri, S., Delneri, A., & Franko, M. (2013). Chitosan-cellulose composite materials: preparation, characterization and application for removal of microcystin. Journal of Hazardous Materials, 252, 355-366.
(23) Vasconcelos, A., & Cavaco-Paulo, A. (2013). The use of keratin in biomedical applications. Current Drug Targets, 14, 612-619.
(24) Verma, V., Verma, P., & Ray, A. R. (2008). Preparation of scaffolds from human hair proteins for tissue-engineering applications. Biomedical Materials, 3, 2500.
(25) Vilaplana, F., Stroemberg, E., & Karlsson, S. (2010). Environmental and resource aspects of sustainable biocomposites. Polymer Degradation and Stability, 95(11), 2147-2161.
(26) Xie, H., Li, S., & Zhang, S. (2005). Ionic liquids as novel solvents for the dissolution and blending of wool keratin fibers. Green Chemistry, 7, 606-608.

### Example 2—One-Pot Synthesis of Biocompatible Silver Nanoparticle Composites from Cellulose and Keratin: Characterization and Antimicrobial Activity

Reference is made to Tran et al., “One-Pot Synthesis of Biocompatible Silver Nanoparticle Composites from Cellulose and Keratin: Characterization and Antimicrobial Activity,” Applied Materials & Interfaces, 2016, 8, 34791-34801, the content of which is incorporated herein by reference in its entirety.

Abstract

A novel, simple method was developed to synthesize biocompatible composites containing 50% cellulose (CEL) and 50% keratin (KER) and silver in the form of either ionic (Ag⁺) or Ag⁰ nanoparticle (Ag+NPs or Ag⁰NPs). In this method, butylmethylimmidazolium chloride ([BMIm⁺Cl⁻]), a simple ionic liquid, was used as the sole solvent and silver chloride was added to the [BMIm⁺Cl⁻] solution of [CEL+KER] during the dissolution process. The silver in the composites can be maintained as ionic silver (Ag⁺) or completely converted to metallic silver (Ag⁰) by reducing it with NaBH₄. Results of spectroscopy (Fourier-transform infrared (FTIR), X-ray diffraction (XRD)) and imaging (scanning electron microscope (SEM)) measurements confirm that CEL and KER remain chemically intact and homogeneously distributed in the composites. Powder X-ray diffraction (XRD) and SEM results show that the silver in the [CEL+KER+Ag⁺] and [CEL+KER+Ag⁰] composites is homogeneously distributed throughout the composites in either Ag⁺ (in the form of Ag₂O nanoparticles (NPs)) or Ag⁰NPs form with size of (9±1) nm or (27±2) nm, respectively. Both composites were found to exhibit excellent antibacterial activity against many bacteria including Escherichia coli, Staphylococus aureus, Pseudomonas aeruginosa, methicillin resistant Staphylococcus aureus (MRSA), vancomycin resistant Enterococcus faecalis (VRE). The antibacterial activity of both composites increases with the Ag⁺ or Ag⁰ content in the composites. More importantly, for the same bacteria and the same silver content, [CEL+KER+Ag⁰] composite exhibits relatively greater antimicrobial activity against bacteria compared to the corresponding [CEL+KER+Ag⁺] composite. Experimental results confirm that there was hardly any Ag⁰NPs release from the [CEL+KER+AgNPs] composite, and hence its antimicrobial activity and biocompatibility is due, not to any released Ag⁰NPs but rather entirely to the Ag⁰NPs embedded in the composite. Both of Ag₂ONPs and Ag⁰NPs were found to be toxic to human fibroblasts at higher concentration (>0.72 mmol), and that for the same silver content, [CEL+KER+Ag₂ONPs] composite is relatively more toxic than [CEL+KER+AgNPs] composite. As expected, by lowering the Ag⁰NPs concentration to 0.48 mmol or less, the [CEL+KER+AgNPs] composite can be made biocompatible while still retaining its antimicrobial activity against bacteria such are E. coli, S. aureus, P. aeruginosa, MRSA, VRE. These results together with our previous finding that [CEL+KER] composites can be used for controlled delivery of drugs such as ciprofloxacin clearly indicate that the [CEL+KER+AgNPs] composite possess all required properties for successfully used as high performance dressing to treat chronic ulcerous infected wounds.

Introduction

Interest in nanoparticles particularly silver nanoparticles (AgNPs) has increased significantly recent years because, among other unique features, the NPs are known to exhibit both antimicrobial and antiviral activities.¹⁻⁸ It has been shown that AgNPs exhibit highly antimicrobial activity against both Gram-positive and negative bacteria.¹⁻⁸ They have also shown to be effective antiviral agent.¹⁻⁹ The size, morphology and stability of NPs are known to strongly affect their antimicrobial and antiviral activity.¹⁻⁸ Colloidal NPs are known to undergo coagulation and aggregation in solution, which, in turn, lead to changes in their size and morphology and hence their antibacterial and antiviral properties. It is, therefore, important to develop an effective and reliable method to anchor the NPs into a supporting material in order to prevent their coagulation and aggregation so that they can maintain their activity. In fact, AgNPs have been encapsulated in various man-made polymers and/or biopolymers, and such systems have been reported to retain some of their antimicrobial and antiviral activity.¹⁻⁸ For example, anchoring AgNPs onto methacrylic acid copolymer beads have proved to be highly effective against a few bacteria.¹⁻¹⁸ However, antimicrobial property of all reported AgNPs-encapsulated composites was tested for only very few bacteria, and more importantly, their biocompatibility has not been determined.¹⁻¹⁸ The lack of the latter information is critical since toxicity of AgNPs is known to be dependent on concentration, and without information on biocompatibility, application of such composite is rather limited. It is, therefore, of particular importance to develop a novel method to anchor AgNPs onto composites biopolymers such as cellulose and keratin, and thoroughly and systematically investigate the antimicrobial and biocompatibility of the composites.

Keratins (KER) are a group of cysteine-rich fibrous proteins found in filamentous or hard structures such as hairs, wools, feathers, nails and horns.¹⁹⁻²⁸ KER possess amino acid sequences similar to those found on extracellular matrix (ECM), and since ECM is known to interact with integrins which enable it to support cellular attachment, proliferation and migration, KER-based materials are expected to have such properties as well.¹⁹⁻²⁸ Furthermore, KER is known to possess advantages for wound care, tissue reconstruction, cell seeding and diffusion, and drug delivery.¹¹⁻²⁰ Unfortunately, in spite of its unique properties, KER has relatively poor mechanical properties, and as a consequence, it was not possible to fully exploit unique properties of keratin for various applications.¹⁹⁻²⁸ To increase the structural strength of KER-based materials, attempts have been made to cross-link KER chains with a crosslinking agent or introduce functional groups on its amino acid residues via chemical reaction(s).¹⁹⁻²⁸ The rather complicated, costly and multistep process is not desirable as it may inadvertently alter its unique properties, making the KER-based materials less biocompatible and toxic, and removing or lessening its unique properties. A new method which can improve the structural strength of KER-based products not by synthetic methods rather by use of naturally occurring polysaccharides such as CEL, is particularly needed.

We have demonstrated recently that a simple ionic liquid (IL), butylmethylimmidazolium chloride ([BMIm⁺Cl⁻]), can dissolve both cellulose (CEL) and KER and by use of this IL as the sole solvent, we developed a simple, GREEN and totally recyclable method to synthesize [CEL+KER] composites just by dissolution without using any chemical modifications or reactions.²⁹⁻³⁵ Spectroscopy (FTIR, NIR, ¹³C CP-MAS-NMR) results indicate that there was no chemical alteration in the structure of CEL and KER.²⁹⁻³⁵While there may be some changes in the molecular weights of CEL and KER, by use of newly developed partial least square regression to analyze FTIR spectra of the [CEL+KER] composites, we found that KER retains some of it secondary structure in the composites.^{31,35} The [CEL+KER] composites obtained were found to retain unique properties of their components, namely, superior mechanical strength from CEL and controlled release of drugs by KER.²⁹⁻³⁵

The information presented clearly indicates that it is possible to use [CEL+KER] as a biocompatible composite to encapsulate AgNPs. Such considerations prompted us to initiate this study which aims to hasten the breakthrough by systematically exploiting advantages of ILs, a green solvent, to develop a novel, simple method to synthesize the [CEL+KER] composite containing silver in either Ag⁺ or Ag⁰ forms. As will be demonstrated, by initially introducing silver salt into the [CEL+KER] composite during the dissolution of CEL and KER by [BMIm⁺Cl⁻], and subsequently reducing the Ag⁺ into Ag⁰NPs directly in the composite, we successfully synthesize the [CEL+KER+Ag⁰NPs] composite. Alternatively, by not carrying out the reduction reaction, we can obtain the [CEL+KER+Ag+NPs] composite. Because the [CEL+KER+Ag⁰NPs] and [CEL+KER+Ag+NPs] composites obtained can prevent the Ag+NPs and Ag⁰NPs from changing size and morphology as well as undergo coagulation, they can, therefore, fully retain the unique property of the silver nanoparticles for repeated use without any complication of reducing activity and not fully recover after each use. With these two composites, we will be able to finally address the important question which, to date, still remains unanswered, namely, the antimicrobial activity of silver nanoparticles due to either Ag⁺ or Ag⁰ or both, and if both forms are active, which NPs have higher activity. We will also systematically investigate biocompatibility of the two composites; information obtained will be used to guide selection and use of the nanoparticle composites. The synthesis, characterization, antimicrobial activity and biocompatibility of the [CEL+KER+Ag+NPs] and [CEL+KER+AgNPs] composites are reported herein.

Experimental Section

Chemicals.

Microcrystalline cellulose (DP=300) and AgCl₂ were from Sigma-Aldrich and used as received. Raw (untreated) sheep wool, obtained from a local farm, was cleaned by Soxhlet extraction using a 1:1 (v/v) acetone/ethanol mixture at 80±3° C. for 48 h. The wool was then rinsed with distilled water and dried at 100±1° C. for 12 h.³⁰⁻³² 1-Methylimidazole and n-chlorobutane (both from Alfa Aesar, Ward Hill, Mass.) were distilled and subsequently used to synthesize [BMIm⁺Cl⁻] using method previously reported.¹⁹⁻³⁵ Nutrient broth (NB) and nutrient agar (NA) were obtained from VWR (Radnor, Pa.). Minimal essential medium (MEM), Fetal Bovine Serum (FBS), and Penicillin-Streptomycin were obtained from Sigma-Aldrich (St. Louis, Mo.), whereas Dulbecco's Modified Eagle Medium (DMEM), PBS, trypsin solution (Gibco) were obtained from Thermo Fischer Scientific (Waltham, Mass.). CellTiter 96® AQueous One Solution Cell Proliferation Assay was obtained from Promega (Madison, Wis.).

Bacterial and Cell Cultures.

The bacterial cultures used were either obtained from the American Type Culture Collection (ATCC, Rockville, Md.) or from the Leibniz Institute DSMZ—German Collection of Microorganisms and Cell Cultures (Braunschweig, Germany). The cell cultures of human fibroblasts were obtained from ATTC (Rockville, Md.).

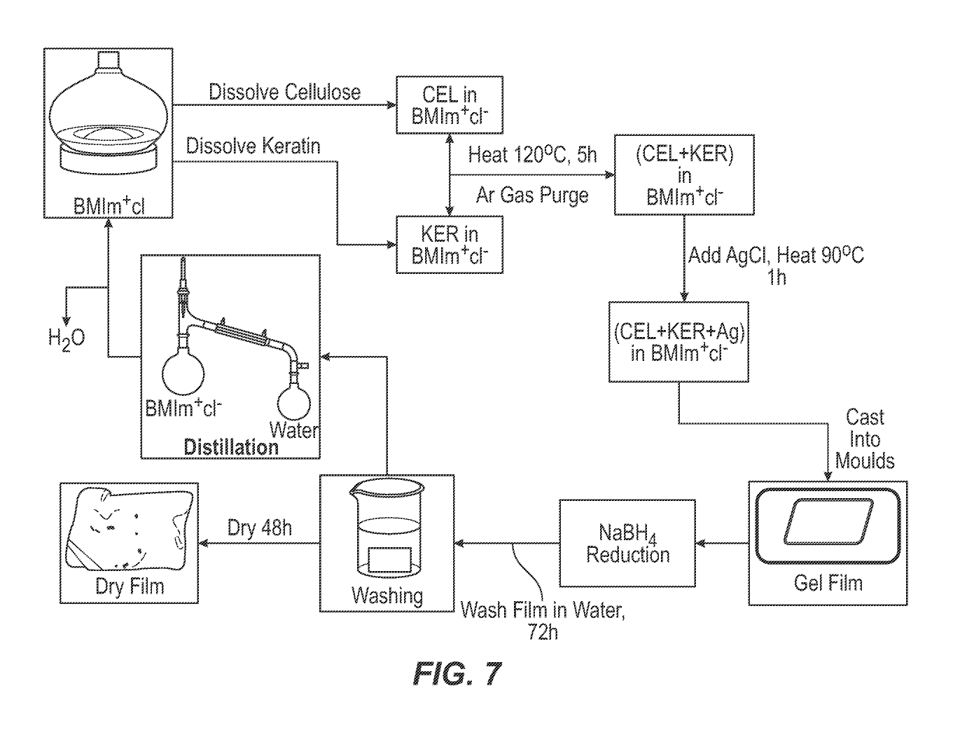

Synthesis.

[CEL+KER+Ag+NPs] and [CEL+KER+Ag⁰NPs] composites were synthesized with minor modification to the procedure we developed previously for the synthesis of [CEL+CS+KER] composites.^{30-32,35} As shown in FIG. 7, washed wool was dissolved in BMIm⁺Cl⁻ at 120° C. Once dissolved, the solution temperature was reduced to 90° C. before CEL was added to the KER solution. Using this procedure, [BMIm⁺Cl⁻] solution of CEL and KER containing up to total concentration of 6 wt % (relative to IL) with various compositions and concentrations were prepared. Concurrently, in a separate flash, AgCl was dissolved in 2 mL of [BMIm⁺Cl⁻], and the mixture will then be added dropped wise to the BMIm⁺Cl⁻ solution of [CEL+KER]. The resulted solution was then casted onto PTFE molds with desired thickness on Mylar films to produce thin composite films with different compositions and concentrations of CEL, KER and Ag⁺. They were then kept in the dark and at room temperature for 24 hrs to allow gelation to yield Gel Films. The Ag⁺ doped Gel Film was then washed with water for 3 days to remove BMIm⁺Cl⁻, and then dried slowly (3-5 days), in the dark at room temperature in a humidity controlled chamber to yield [CEL+KER+Ag+NPs] composite. Alternatively, the Ag⁺ doped Gel Film was reduced with NaBH₄ to Ag⁰NPs. For example, the Gel Film, sandwiched between two PTFE meshes, was placed in an aqueous solution of either NaBH₄, in the dark and at room temperature for 48 hrs. Subsequently, the reduced film was washed and dried slowly (˜3-5 days) in the dark and at room temperature in a humidity-controlled chamber to yield [CEL+KER+AgNPs] composite.

Analytical Characterization.

FTIR spectra (from 450-4,000 cm⁻¹ were recorded on a Spectrum 100 Series FTIR spectrometer (Perkin Elmer, USA) at resolution of 2 cm⁻¹ by the KBr method. Each spectrum was an average of 64 individual spectra. X-ray diffraction (XRD) measurements were taken on a Rigaku MiniFlex II diffractometer utilizing the Ni filtered Cu Kα radiation (1.54059 Å). The voltage and current of the X-ray tube were 30 kV and 15 mA respectively. The samples were measured within the 20 angle range from 2.0 to 40.00. The scan rate was 5° per minute. Data processing procedures were performed with the Jade 8 program package.²⁹⁻³⁵ The surface and cross-sectional morphologies of the composite films were examined under vacuum with a JEOL JSM-6510LV/LGS Scanning Electron Microscope with standard secondary electron (SEI) and backscatter electron (BEI) detectors. Prior to SEM examination, the film specimens were made conductive by applying a 20 nm gold-palladium-coating onto their surfaces using an Emitech K575x Peltier Cooled Sputter Coater (Emitech Products, TX).

In Vitro Antibacterial Assays.

The antibacterial characteristics of the newly synthesized composites were tested against E. coli (ATCC 8739, DSMZ 498), Staphylococcus aureus (ATCC 25923, DSMZ 1104), methicillin resistant S. aureus (ATCC 33591, DSMZ 11729), vancomycin resistant Enterococcus faecalis (ATCC 51299, DSMZ 12956), and Pseudomonas aeruginosa (ATCC 9027, DSMZ 1128) using previously published protocol.^{29,33,34} The cultures were grown in a sterile nutrient broth medium overnight at 37° C. and 150 rpm. Composites of dimensions of 3×20 mm were prior to the assay thermally sterilized at 121° C., 15 psi for 20 min. They were placed in a diluted overnight culture (2 μL of overnight culture in 2 mL of nutrient broth) and incubated for 24 hours at 37° C. and 200 rpm. Bacteria were plated in serial dilutions onto sterile nutrient agar plates at time 0 and after 24 hours, and incubated overnight at 37° C. Colony forming units (CFUs) were quantified on statistically significant plates (30-300 CFUs) and compared to a control (no added material). Log of reduction of bacteria as follows was calculated for each experiment:

$$Log of reduction = log \frac{{N}_{0}}{{N}_{t}}$$

where N₀ is the number of bacteria at the beginning of the experiment, and Nₜ is the number of bacteria after 24 hours.

In Vitro Biocompatibility Assays.

The biocompatibility of [AgNPsCEL:KER] composites was assessed by the adherence and growth of fibroblasts in the presence of the composites, as previously reported.^{29,33,34} Human fibroblasts (ATCC CRL-2522 or ATCC CCL-186) were grown in a sterile minimal essential medium (MEM) or in a sterile Dulbecco's Modified Eagle Medium (DMEM) supplemented with 10% FBS and 1% Penicillin-Streptomycin according to ATCC guidelines. The inoculated culture was grown at 37° C. in a humidified atmosphere of 5% CO₂ until the 3^{rd} passage. Between passages cells at approximately 80% confluency were subjected to trypsinization and recovered by centrifugation at 1000 g for 10 min. The cell pellets were resuspended homogenously into the culture media and transferred into a 75 cm² tissue culture flask for further passages. Cells were seeded into the wells of the 24-well plate at a concentration of 2×10⁴ cells/mL and left for 1 day to allow for their attachment (approximately 50% confluency). Circle-shaped composites with either 15 or 7 mm in diameter were autoclaved at 121° C., 15 psi for 20 min and placed into the wells with attached cells the following day. Some wells contained cells without any added material and served as a control. After incubating for 3 days, viability and fitness of the cells was evaluated both, with a colorimetric CellTiter 96® AQueous One Solution Cell Proliferation Assay, and visually with an Olympus DP12 digital microscope camera and. The procedure for the CellTiter 96® AQueous One Solution Cell Proliferation Assay was followed as specified in the manufacturer's manual. In brief, the MTS reagent was added in a 1:5 ratio to each well after the medium in wells was supplemented with a colorless MEM or colorless DMEM. The cells were then incubated at standard culture conditions for 3 h. Then 100 μL from each well was transferred to a new 96-well cell culture plate and optical density (OD value) of the extracted supernatant was measured with a Perkin Elmer HTS 7000 Bio Assay Reader at 490 nm. The percent viability was calculated using the following equation:

$$\% cell viability = \frac{{OD}_{Test sample}}{{OD}_{Control}} \times 100$$

where OD_{Test sample} is the measured OD at 490 nm of the extract from the test sample well, and OD_{Control} is the measured OD at 490 nm of the extract from the control well.

Measurements of Ag⁰NPs Released from [CEL+KER+Ag⁰NPs] Composites by Thermal Lens Method.

Any possible AgNPs released from the composite materials was determined using the previously developed method. In this method, AgNPs were detected by measuring their surface plasmons resonance band at 409 nm by the thermal lens technique in a flow injection analysis (FIA). As described in the Experimental Section, AgNPs were produced by reducing Ag⁺ with sodium borohydride, there is a remote possibility that some minute amount of Ag⁺ may remained unreduced and remained in the composites (even though XRD results indicate that no Ag⁺ is present in the composite) which was subsequently released. Because this thermal lens detection technique cannot detect any released Ag⁺ as it does not have any surface plasmon resonance absorption, any released Ag⁺ was converted into AgNPs by sodium borohydride directly by use of the FIA so that they can be readily detected. As a consequence, results obtained will provide information on two concentrations: colloidal silver concentration or (concentration of released AgNPs) and total silver concentration which is the sum of released AgNPs concentration plus released Ag⁺ concentration.

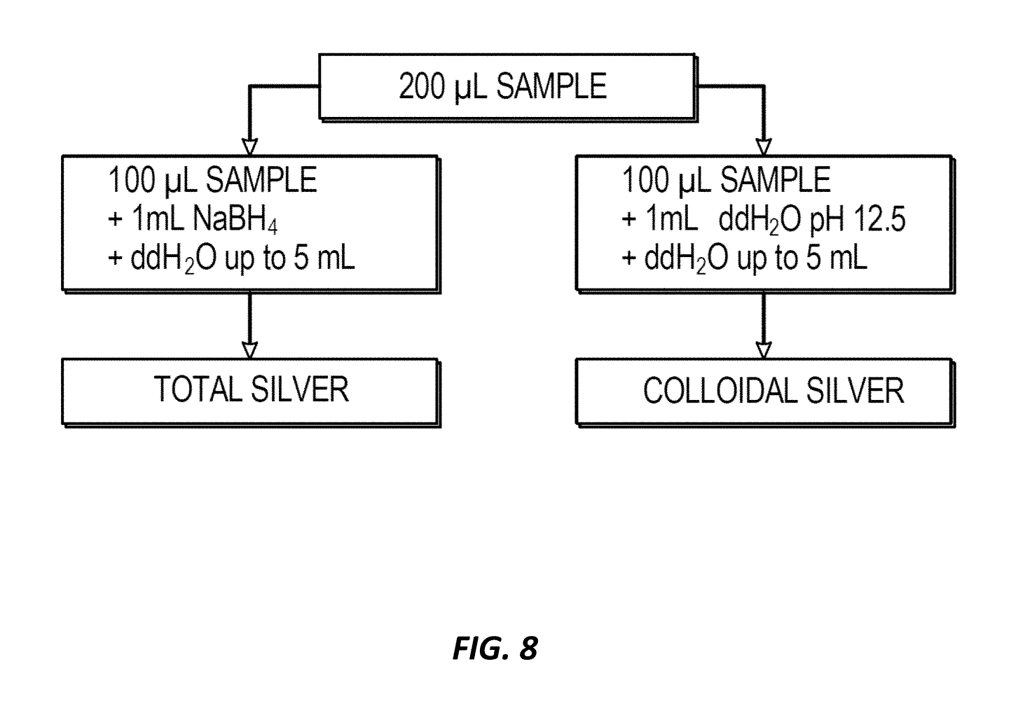

The experimental setup to measure silver release mirrored the experimental setup used in bioassays. Composite materials of dimensions 3×20 mm² were put in sterile falcon tubes with 2 mL of sterile 1×PBS at pH 7.4. Three replicates each of blank samples ([CEL+KER]) and [CEL+KER+500 mg Ag⁰NPs] composites were used. Tubes were put on a shaker at 400 rpm and kept at 37° C. in darkness for 7 days. Samplings were conducted at time 0, 24 hrs, 3 days and 7 days. At every sampling 200 μL of sample was taken out of each tube and replaced with 200 μL of fresh PBS. The dilution was taken into account when calculating final concentrations. 100 μL of sample was reduced with 0.60 mM sodium borohydride (NaBH₄) in order to measure total silver (AgNPs+Ag⁺), whereas the other 100 L of sample was not reduced in order to measure only colloidal silver (AgNPs) released from the sample. Sample preparation was done as shown in FIG. 8.

Sample preparation was done in glass tubes wrapped in aluminum foil to protect it from light. Dilution made at sample preparation was taken into account when calculating measured concentrations.

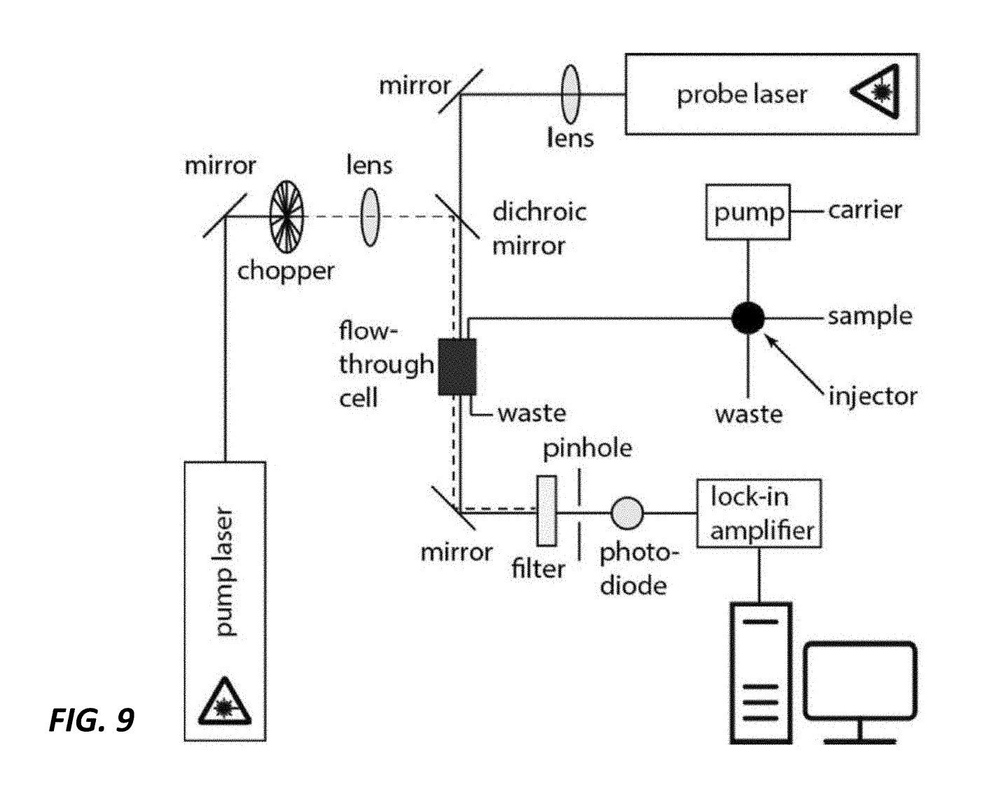

All measurements were conducted on an in-house-built FIA system with a dual beam TLS detection unit.^{51,52} The instrumental setup is schematically presented in FIG. 9. Krypton laser operating at 407 nm (150 mW power) was used as a source of the pump-beam. The emission of a He—Ne laser (632.8 nm, 2 mW) served as a probe beam. The pump-beam modulation frequency was 40 Hz. Flow rate of the carrier (dd H₂O) was 0.600 mL/min.

Sample was injected through the metal free injection valve, equipped with a 100 μL PEEK sample loop. Separate calibration curve was prepared every time a set of samples was measured. Limit of detection (LOD) for this method was calculated as follows:

$$LOD = \frac{3 \cdot {SD}_{blank}}{k}$$

where SD_{blank} corresponds to standard deviation of blank signal, and k is the slope of the calibration curve. To further confirm that the signals obtained are from the Ag0NPs released from the [CEL+KER+Ag⁰NPs] composites, additional experiment was designed in which nitric acid (HNO₃) was added to the released sample solution to dissolve the released Ag⁰NPs. Specifically, 2.0 μL of concentrated HNO₃ was added to 6 mL of released sample to dissolve the Ag⁰NPs. The Ag⁺ obtained was then reconverted back to Ag⁰NPs by addition of 6.0 mL PBS (pH 12.5) and 600 μL 0.6 mM NaBH₄ to 6 mL of dissolved sample. Samples at each stage of the experiment (before dissolution, after dissolution, and after recovery) were measured on the FIA thermal lens setup described above using the same conditions.

Statistical Analysis.

All experiments had sample size of n=3 and are representative of repeated trials. Sample error bars on plots represent±standard error of mean (SEM), where applicable. Tests for statistical significance of the difference of the means were performed using a two-tailed Student's t-test assuming unequal variances using Microsoft Office Excel. P-values are indicated as follows on figures: (*P<0.05); (**P<0.005); (***P<0.001).

Results and Discussion

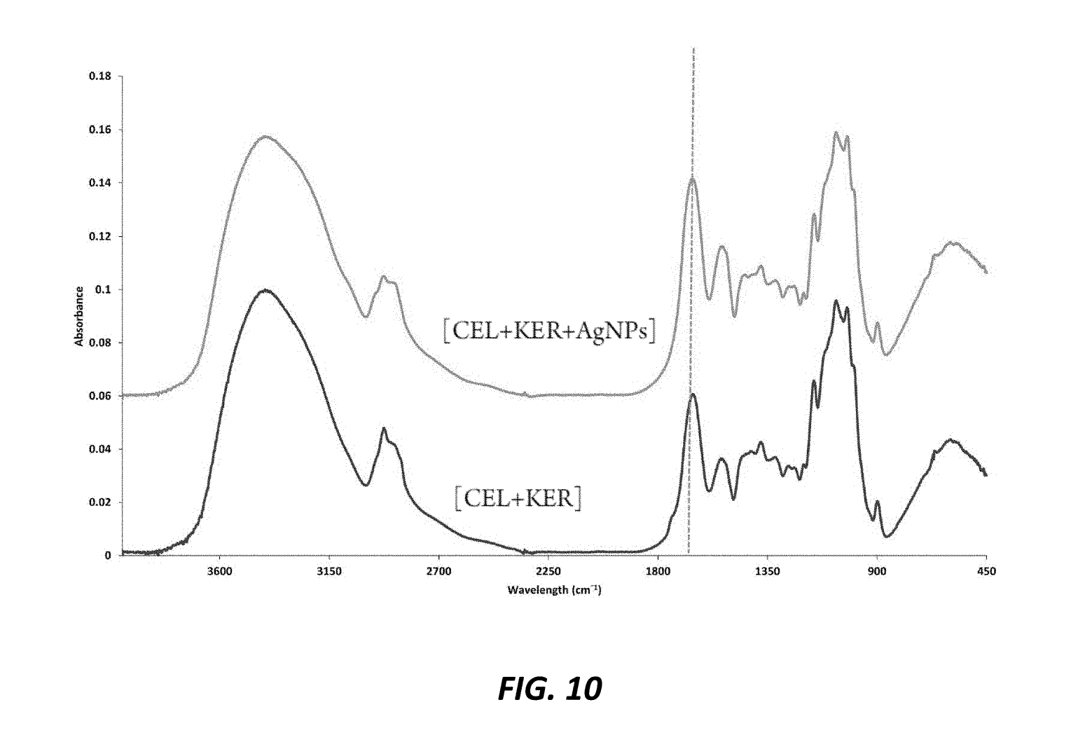

FTIR. FTIR spectrum of the [CEL+KER+Ag⁰NPs] composite is presented as the orange spectrum in FIG. 10. For reference, spectrum of the [CEL+KER] composite is also added (blue spectrum). As expected, the blue spectrum of the [CEL+KER] is similar to those previously observed for the [CEL+KER] composites, namely bands at 1700-1600 cm⁻¹ and 1550 cm⁻¹ are due to amide C═O stretch (amide I) and C—N stretch (amide II) vibrations, and at 1300-1200 cm⁻¹ are from the in-phase combination of the N—H bending and the C—N stretch vibrations (amide III).^{30-32,36-38}, Major bands between 1200- and 900-cm⁻¹ are due to sugar ring deformations of the CEL.^{30-32,36-38} The fact that the orange spectrum of the [CEL+KER+AgNPs] composite is relatively similar to the green spectrum of the [CEL+KER] composite seems to indicate that there may not be strong interaction between the Ag⁰NPs and CEL and KER in the composite. However, careful inspection of the spectra revealed that there are indeed minor differences in the amide bands at around 1700-1600 cm⁻¹ and 1550 cm⁻¹ between the two spectra. Specifically, interaction between Ag⁰NP and C═O group leads to the shift in the amide band at 1650 cm⁻¹ (of the [CEL+KER] composite) to 1655 cm⁻¹ (of the [CEL+KER+Ag⁰NP] composite). Also, the small shoulder at ˜1449 cm⁻¹disappears upon adding Ag⁰NP to the composite. These results seem to indicate that there may be some interactions between the Ag⁰NP and the amide groups of the KER. Furthermore, difference of the band at ˜2870 cm⁻¹ between the spectra of the two composites suggests that there may be some modifications in the hydrogen bonding when Ag⁰NP was incorporated into the [CEL+KER] composite.³⁰⁻³²

Powder X-Ray Diffraction (XRD).

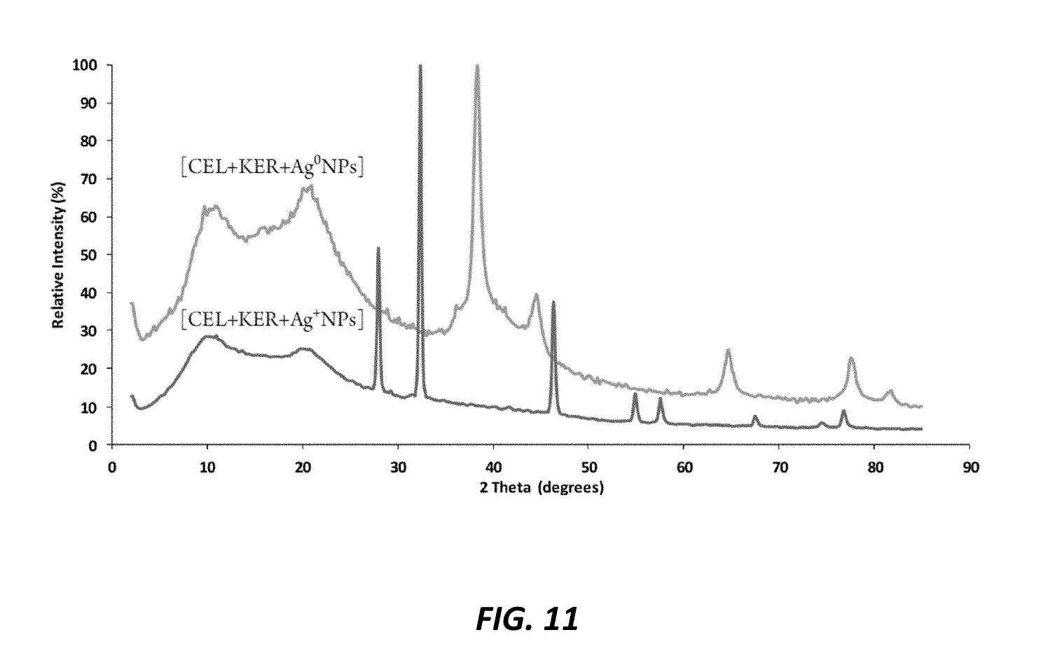

X-ray diffractograms of [CEL+KER+Ag⁺ NPs] and [CEL+KER+Ag⁰NPs] composites are shown in FIG. 11. Because CEL and KER are present in both composites, it is as expected that both spectra have similar two broad bands at around 2θ=0.75° and 20.85° which are due to CEL and KER. Since the valency of the silver nanoparticles is different in the composites, narrow crystalline bands which are due to the silver nanoparticles are distinctly different for the two composites. Specifically, the diffractogram of [CEL+KER+Ag⁺ NPs] composite (blue spectrum) exhibits three major peaks at (2θ)=27.94°, 32.35° and 46.37° which are characteristic of the (1 1 0), (1 1 1) and (2 1 1) peaks, respectively, of silver oxide nanoparticles (Ag₂ONPs).³⁹⁻⁴³ The fact that these peaks are the same as those previously reported for Ag₂O NPs⁴⁰⁻⁴³ as well as the reference diffractogram of Ag₂O reported in the JCPDS file No 42-0874 seems to indicate that Ag⁺ reacted with oxygen to form Ag₂O following by aggregation to form Ag₂ONPs. Conversely, the diffraction peaks at 38.47°, 44.57°, 64.87° and 77.66° in the orange spectrum of the [CEL+KER+AgNPs] composite can be attributed to the (1 1 1), (2 0 0), (2 2 0) and (3 1 1) bands of Ag⁰.⁴⁴⁻⁴⁶ The fact that there is no diffraction peak of Ag⁰ in the [CEL+KER+Ag⁺ NPs] suggests that this composite contains only silver oxide nanoparticles. Similarly, since there is no peak due to Ag₂ONPs is seen in the diffractogram of the [CEL+KER+AgNPs] composite, it is reasonable to infer that silver ion was completely reduced to metallic silver nanoparticles during the synthesis.

Scherrer equation was then used to determine the size (t value) of the Ag₂ONPs and Ag⁰NPs in the composites from the full width at half maximum (FWHM, 3 value in the equation) of their corresponding XRD peaks:^{47,48}

$$τ = \frac{k λ}{βcosθ}$$

where τ is the size of the nanoparticle, λ is the X-ray wavelength and k is a constant.^{31,32} The size of the metallic silver nanoparticle in the [CEL+KER+Ag⁰] composite was found to be (9±1) nm while the Ag₂ONPs in the [CEL+KER+Ag⁺] composite has the size of (27±2) nm. It is unclear why the size of the silver oxide is much larger than that of the metallic silver NPs. It may be possible that the stirring and reducing with NaBH₄ further dispersed the silver ion NPs in the [CEL+KER] composite thereby preventing them from coagulation upon reducing to Ag⁰NPs.

Scanning Electron Microscope (SEM) Images and Energy Disperse Spectroscopy (EDS) Analysis.

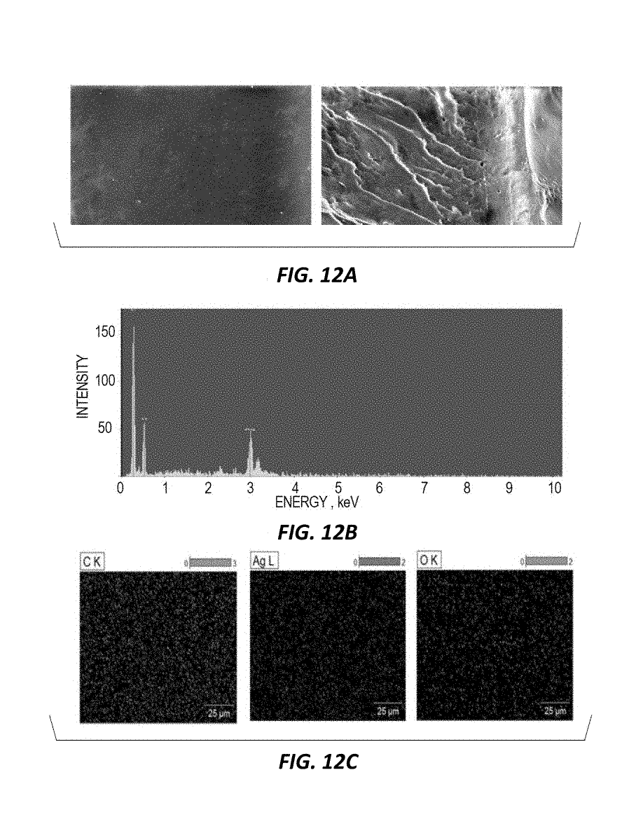

Shown in FIG. 12A are surface (left) and cross section SEM images of the [CEL+KER+AgNPs] composite. As expected, the images of the composite are similar to those we previously observed for the [CEL+KER] composites.³⁰⁻³² That is CEL and KER are homogeneously distributed throughout the composite. While CEL is known to have rather smooth structure, the presence KER in the composite gives it a rough and porous structure with a three-dimensional interconnection throughout the film. More information on the chemical composition and homogeneity of the composite can be seen in FIGS. 12B and 12C which show the EDS spectrum of the composite (3B) and images taken with EDS detector specifically set for carbon (3C left), silver (3C center) and oxygen (3C right). As evident from FIG. 12C, the silver nanoparticles were not only well incorporated into the composites, but were also present as well distributed nanoparticles throughout the composite.

Antibacterial Assay.

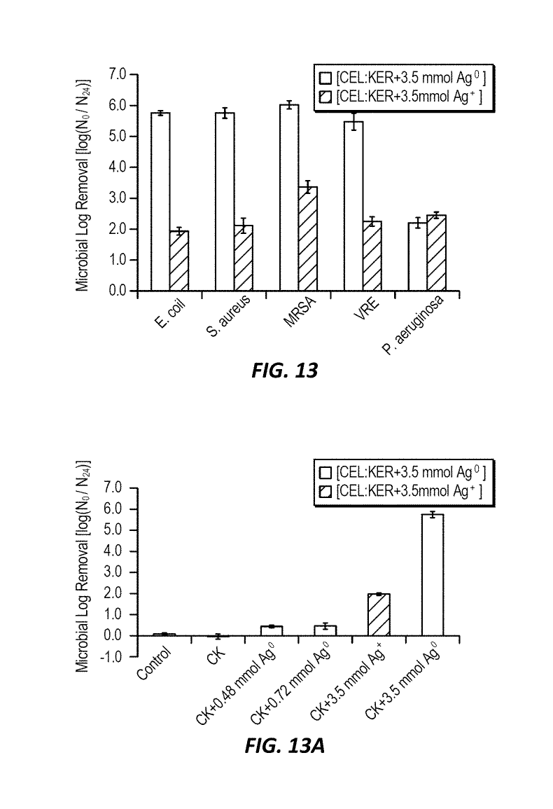

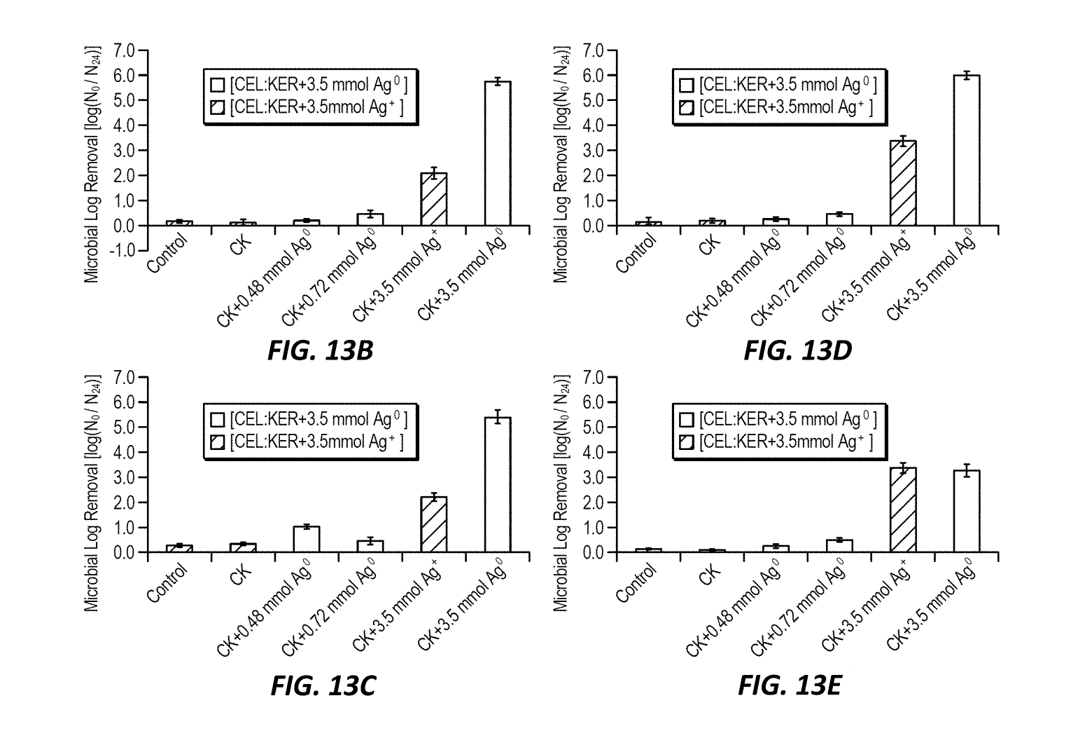

To assess the antimicrobial effect of AgNPs in the [CEL+KER+AgNPs] composites, bacteria were grown in the presence of the composites and then plated out onto nutrient agar and measured by the number of colonies formed compared to those for the blank ([CEL+KER] composite) and the control (no composite). Results for the microbial log of reduction of different composites are shown in both FIG. 13 top (for composites with 3.5 mmol of either Ag⁺ or Ag⁰) and bottom A-E (for NPs with three different concentrations: 3.5 mmol, 0.72 mmol and 0.48 mmol). It is evident that bactericidal activity of [CEL+KER+AgNPs] composites increases with the concentration of silver NPs in both Ag⁰ and Ag⁺ forms for all bacteria tested. Specifically, as shown in FIG. 13 top, [CEL+KER+Ag⁰] composites (black bar) with 3.5 mmol of silver exhibited the highest bactericidal activity against all selected bacteria with up to 6-logs of reduction in number of bacteria, which corresponds to 99.9999% growth reduction. Even at silver concentration as low as 0.48 mmol, the composite still exhibited up to 0.5-logs of reduction, or 68% growth reduction for most of bacteria, with the exception of VRE, where 1-log of reduction was observed (FIG. 13 bottom A-E). As expected, controls and blank samples (light grey bars) did not exhibit any statistical significantly reduction in number of bacteria, and there was no significant difference between them.

While it is known that AgNPs are bactericidal, to date, it is still unclear if the antimicrobial activity is due to Ag⁰ or Ag⁺ (as in Ag₂O). As described above, by judiciously selecting the synthetic method, the [CEL+KER+AgNPs] can be synthesized with the silver NPs in either Ag⁰ or Ag⁺ form. This makes it possible, for the first time, to elucidate the mechanism of antimicrobial activity of AgNPs. Accordingly, microbial assays were carried out in the presence of either [CEL+KER+Ag⁰NPs] composites (black bars) or [CEL+KER+Ag⁺] composites (hatched bars). Results obtained, shown in both FIG. 13 top and bottom, clearly show that for the same bacteria and the same silver content, [CEL+KER+Ag⁰] composites (black bars) exhibit relatively greater antimicrobial activity against bacteria compared to the corresponding [CEL+KER+Ag⁺] composites (hatched bars). For example, as shown in FIG. 13 bottom 13A-D, up to 6-log of reduction of growth was found by [CEL+KER+Ag⁰NPs] composite for all four bacteria (E. coli, S. aureus, MRSA and VRE) whereas [CEL+KER+Ag⁺] composite exhibits only 3.5-log of reduction. Surprisingly, within experimental errors, there was no significant difference between these two nanoparticle composites for P. aeruginosa (FIG. 13E). Results obtained also indicate that [CEL+KER+Ag⁰NPs ] composites not only have relatively stronger antimicrobial activity compared to corresponding [CEL+KER+Ag⁺] composites, but that the rather limited antimicrobial activity of the latter cannot be enhanced by increasing the concentration of Ag⁺ in the composites because, as will be shown in the following section, Ag⁺ is not biocompatible and as a consequence, increasing Ag⁺ concentration would undesirably lead to damaging and killing human cells Again, as expected, there was no statistically significant decrease in number of bacteria after 24 hours in control experiments (no composite) and blank samples.

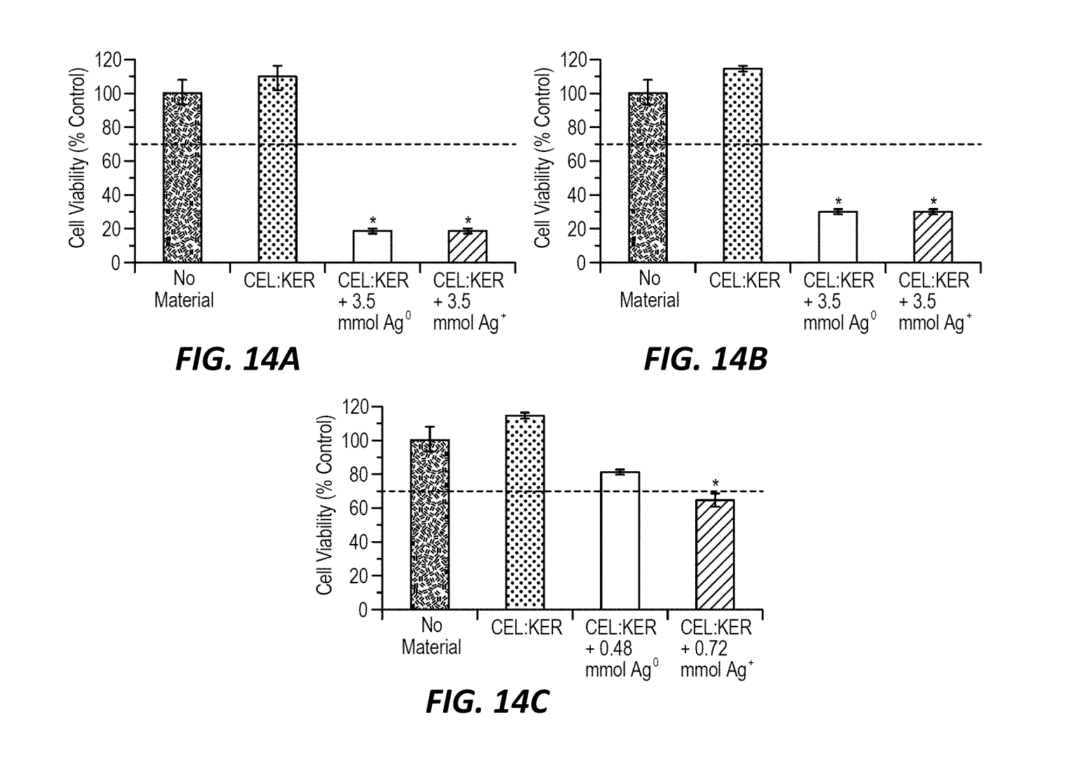

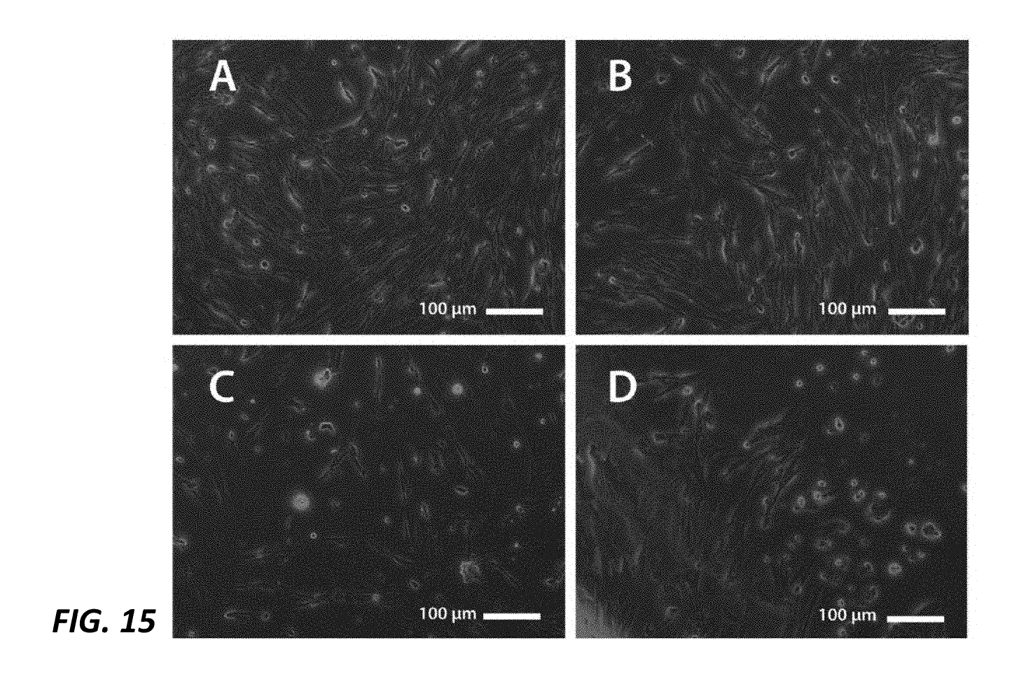

Biocompatibility Assay. To assess a potential cytotoxicity of the [CEL+KER+AgNPs] composites with different concentrations of silver NPs, the morphology and the proliferation capabilities of adherent human fibroblasts in presence or absence of the nanoparticle composites were analyzed. The proliferation capability was assessed using a colorimetric assay CellTiter 96® AQueous Non-Radioactive Cell Proliferation Assay (or CellTiter 96® AQueous One Solution Cell Proliferation Assay), whereas the morphology of fibroblasts was examined microscopically. Three trials were performed for this assay, employing composites with different sizes (circle of either 15 or 7 mm diameter) and silver concentrations. Fibroblasts were exposed to the composites for 3 days. Proliferation and viability of fibroblasts in the presence or absence of the composites with different concentrations of AgNPs over time 3 days is shown in FIG. 14. Statistical significance in differences between the sample wells and control wells were evaluated with two-tailed student's t-test, and the degree of significance is indicated with P values in different significance levels (alpha=0.05, 0.005, or 0.001). In the first trial, the composites of 15 mm diameter and with either 3.5 mmol of Ag⁺ or Ag⁰ concentration were tested (FIG. 14A). The fibroblasts in contact with either the 3.5 mmol [CEL+KER+Ag] or the 3.5 mmol [CEL+KER+Ag⁺] exhibited low absorbances at 490 nm, indicating that cells were not viable. Morphological data obtained through microscopic examination indicated that the fibroblasts in these wells were not attached and exhibit unusual round morphology (data not shown). This seems to indicate that the cells were not healthy and possibly not viable. To reduce the concentration of silver NPs in the composites, in the second trial, the diameter of composites used was reduced from 15 mm to 7 mm which corresponds to 4.6 reduction in the area of the composites. As shown in FIG. 14B, cells in the sample wells exhibited slightly increased viability after 3 days compared to that in the first trial. Morphological data showed round unattached cells (data not shown). Because results obtained so far indicate that the biocompatibility of the [CEL+KER+Ag⁰] composites are relatively better than that of the corresponding [CEL+KER+Ag⁺] composites, subsequent experiments were carried out using only the former. Specifically, [CEL+KER+Ag⁰] composites with relatively lower Ag⁰NPs concentrations (0.48 mmol and 0.72 mmol) were used, (FIG. 14C). In this case, the viability of cells in the composite wells after 3 days of exposure was high, approximately 83% for 0.48 mmol of Ag⁰NPs and (64±5) % for 0.72 mmol of Ag⁰NPs compared to control. It is evidently clear that within experimental error, there was no statistically significant difference between cells in the 0.72 mmol Ag⁰NPs well and 0.48 mmol Ag⁰NPs well and that in the control well. Morphological data presented as images of cells in the 0.48 mmol Ag⁰NPs well (FIG. 15C) and in the 0.72 mmol Ag⁰NPs well (FIG. 15D) show a mix of healthy-looking cells and round unattached cells, similar to those observed for cells in the absence of composite (FIG. 15A) and with [CEL+KER] composite (FIG. 15B). Taken together, the results clearly indicate that both Ag⁺ and Ag⁰NPs are toxic to human fibroblasts at higher concentration (>0.72 mmol). At the same concentration, Ag⁺ is relatively more toxic than Ag⁰. More importantly, at or below the silver concentration of 0.48 mmol, the [CEL+KER+AgNPs] composite is not only fully biocompatible but also fully retains its antimicrobial activity against bacteria such as E. coli, S. aureus, P. aeruginosa, MRSA, VRE.

Release of Ag0NPs from the [CEL+KER+Ag0NPs] Composites.

We also carried out experiments to determine if any Ag⁰NPs are leaking out from the [CEL+KER+AgNPs] composites during the microbial and biocompatibility assays. Such information is particularly important as it would clarify the mechanism of antibacterial activity and biocompatibility of the composites. That is the activity is due either to the Ag⁰NPs in the composites and/or Ag⁰NPs released from the composites. As described in the experimental section, since the [CEL+KER+AgNPs] composites were exhaustedly washed with water for a total of up to 10 days, it is expected that if there is any leaking of silver NPs from the composites, their concentration should be extremely low. Accordingly, we used a modified version of the recently developed ultrasensitive method based on the thermal lens technique to determine the concentration of any possible leaking of the Ag⁰NPs from the composites during the bioassay.^{49,50} No experiment was carried out to measure release of Ag⁺ form the [CEL+KER+Ag⁺] composites because compared to the [CEL+KER+Ag⁰NPs] composites the [CEL+KER+Ag⁺] composites are not readily usable as they are not biocompatible and have relatively lower antimicrobial activity. This thermal lens detection method is so sensitive that it can detect released silver NPs at concentration as low as 0.51 μg/L.³³ As described in the Experimental Section above, two different concentration values can be obtained from this method: colloidal silver concentration or concentration of released Ag⁰NPs, and total silver concentration which is the sum of the released Ag⁰NPs concentration plus released Ag⁺ concentration. As described above, XRD results show that there is no Ag⁺ in the [CEL+KER+AgNPs] composites; i.e., all Ag⁺ was reduced by NaBH₄ to Ag⁰NPs during the preparation. However, there is a possibility that concentration of Ag⁺ remained in the composites was so low that it cannot be detected by XRD. Because this thermal lens detection is so sensitive that it can detect any Ag⁺ that is released from the Ag⁺ remaining in the composites.

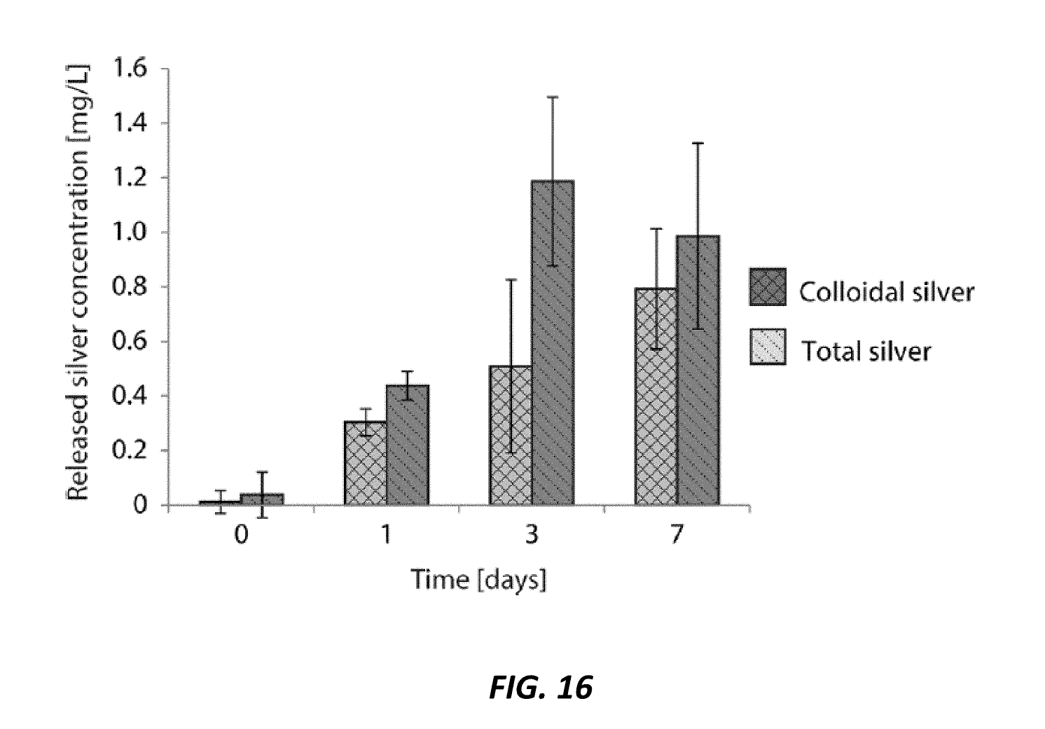

Results obtained, presented in FIG. 16 and plotted as concentration of released silver against time the composites were immersed in the solution similar to the media used in the microbial and biocompatibility assays. The fact that, within experimental errors, and at all times (from the beginning to 7 days), obtained concentration of released Ag⁰NPs (black bars) was the same as that of the total concentration of released silver (grey bars) clearly indicates that all released silver were Ag⁰NPs, there was no Ag⁺ released from the composites. Also, concentrations of released Ag⁰NPs after 3 days were the same, within experimental errors, to those after 7 days indicate that no more Ag⁰NPs was released beyond 3 days. More importantly, even after reaching a plateau at about 3 days and continued beyond 7 days, only 2.3 μg of Ag⁰NPs was released from [CEL+KER+Ag⁰NPs]. Since the total concentration of silver in the composite used in the measurements was about 12 mg, there was less than 0.02% of Ag⁰NPs was released from the [CEL+KER+AgNPs] composites even after they were soaked in the solution for 7 days. Taken together, the results obtained clearly indicate that there was hardly any Ag⁰NPs release from the [CEL+KER+AgNPs] composite, and hence its antimicrobial activity and biocompatibility is due, not to any released Ag⁰NPs but rather entirely to the Ag⁰NPs embedded in the composite.

Conclusions

In summary, we have shown that biocompatible composites containing 50% CEL and 50% KER and silver either in the ionic (Ag⁺, presented as Ag₂ONPs) or metallic (Ag⁰NPs) were successfully synthesized in a simple process in which [BMIm⁺Cl⁻], an simple ionic liquid, was used as the sole solvent, and AgCl was added to the [BMIm⁺Cl⁻] solution of [CEL+KER] during the dissolution process. The silver in the composite can be maintained as Ag⁺ or completely converted to Ag⁰NPs by reducing it with NaBH₄. Results of spectroscopy (FTIR, XRD) and imaging (SEM) measurements confirm that CEL and KER remain chemically intact and homogeneously distributed in the composites. XRD and SEM results show that the silver in the [CEL+KER+Ag⁺] and [CEL+KER+Ag⁰] composites are homogeneously distributed throughout the composites in either Ag₂O NPS or Ag⁰NPs form with size of (9±1) nm or (27±2) nm, respectively. Both composites were found to exhibit excellent antibacterial activity against many bacteria including E. coli, S. aureus, P. aeruginosa, MRSA, VRE. The bacterial activity of both composites increases with the Ag⁺ or Ag⁰NPs content in the composites. More importantly, for the same bacteria and the same silver content, [CEL+KER+AgNPs] composite exhibits relatively greater antimicrobial activity against bacteria compared to the corresponding [CEL+KER+Ag⁺] composite. Experimental results confirm that there was hardly any Ag⁰NPs release from the [CEL+KER+AgNPs] composite, and hence its antimicrobial activity and biocompatibility is due, not to any released Ag⁰NPs but rather entirely to the Ag⁰NPs embedded in the composite. Both of Ag⁺ and Ag⁰NPs were found to be toxic to human fibroblasts at higher concentration (>0.72 mmol), and that for the same silver content, [CEL+KER+Ag⁺] composite is relatively more toxic than [CEL+KER+AgNPs] composite. As expected, by lowering the Ag⁰NPs concentration to 0.48 mmol or less, the [CEL+KER+AgNPs] composite is biocompatible while still retaining antimicrobial activity against bacteria such as E. coli, S. aureus, P. aeruginosa, MRSA, VRE. These results together with our previous finding that [CEL+KER] composites can be used for controlled delivery of drugs such as ciprofloxacin clearly indicate that the [CEL+KER+AgNPs] composite possess all required properties for successfully used as high performance dressing to treat chronic ulcerous infected wounds.

### REFERENCES

(1) Yang, X.; Yang, M.; Pang, B.; Vara, M.; Xia, Y. Gold Nanomaterials at Work in Biomedicine. Chem. Rev. 2015, 115, 10410-10488.
(2) Xia, X.; Zeng, J.; Zhang, Q.; Moran, C. H.; Xia, Y. Recent Developments in Shape-Controlled Synthesis of Silver Nanocrystals. J. Phys. Chem. C 2012, 116, 21647-21656.
(3) Chan, W. C. W.; et al. A Year for Nanoscience. ACS Nano 2014, 8, 11901-11903.
(4) Pelaz, B.; et al. The State of Nanoparticle-Based Nanoscience and Biotechnology: Progress, Promises, and Challenges. ACS Nano 2012, 6, 8468-8483.
(5) Sardar, R.; Shumaker-Parry, J. S. Spectroscopic and Microscopic Investigation of Gold Nanoparticle Formation: Ligand and Temperature Effects on Rate and Particle Size. J. Am. Chem. Soc. 2011, 133, 8179-8190.
(6) Wang, Z.; Bharathi, M. S.; Hariharaputran, R.; Xing, H.; Tang, L.; Li, J.; Zhang, Y.-W.; Lu, Y. pH-Dependent Evolution of Five-Star Gold Nanostructures: An Experimental and Computational Study. ACS Nano 2013, 7, 2258-2265.
(7) Wright, A. R.; Li, M.; Ravula, S.; Cadigan, M.; El-Zahab, B.; Das, S.; Baker, G. A.; Warner, I. M. Soft- and Hard-Templated Organic Salt Nanoparticles with the Midas Touch: Gold-Shelled NanoGUMBOS. J. Mater. Chem. C 2014, 2, 8996-9003.
(8) Takagai, Y.; Miura, R.; Endo, A.; Hinze, W. L. One-pot Synthesis with in situ Preconcentration of Spherical Monodispersed Gold Nanoparticles using Thermoresponsive 3-(alkyldimethylammonio)-propyl sulfate Zwitterionic Surfactants. Chem. Commun. 2016, 52, 10000-10003.
(9) Trefry, J. C.; Wooley, D. P. Silver Nanoparticles Inhibit Vaccinia Virus Infection by Preventing Viral entry through a Macro-pinocytosis-dependent Mechanism. J. Biomed. Nanotechnol. 2013, 9, 1624-1635.
(10) Noh, H. J.; Im, A.-R.; Kim, H.-S.; Sohng, J. K.; Kim, C.-K.; Kim, Y. S.; Cho, S.; Park, Y. Antibacterial Activity and Increased Freeze-drying Stability of Sialyllactose-reduced Silver Nanoparticles using Sucrose and Trehalose. J. Nanosci. Nanotechnol. 2012, 12, 3884-3895.
(11) Guzman, M.; Dille, J.; Godet, S. Synthesis and Antibacterial Activity of Silver Nanoparticles against Gram-positive and Gram-negative Bacteria. Nanomedicine 2012, 8, 37-45.
(12) Mallakpour, S.; Dinari, M.; Talebi, M. A Facile, Efficient, and Green Fabrication of Nanocomposites based on L-leucine Containing Poly(amide-imide) and PVA-modified Ag Nanoparticles by Ultrasonic Irradiation Colloid. Colloid Polym. Sci. 2015, 293, 1827-1833.
(13) Gangadharan, D.; Harshvardan, K.; Gnanasekar, G.; Dixit, D.; Popat, K. M.; Anand, P. S. Polymeric Microspheres Containing Silver Nanoparticles as a Bactericidal Agent for Water Disinfection. Water Res. 2010, 44, 5481-5487.
(14) Wei, D.; Sun, W.; Qian, W.; Ye, Y.; Ma, Y. The Synthesis of Chitosan-based Silver Nanoparticles and their Antibacterial Activity. Carbohydr. Res. 2009, 344, 2375-2382.
(15) Johnston, J. H.; Nilsson, T. Nanogold and Nanosilver Composites with Lignin-containing Cellulose Fibres. J. Mater. Sci. 2012, 47, 1103-1112.
(16) Wu, J.; Zheng, Y.; Song, W.; Luan, J.; Wen, X.; Wu, Z.; Chen, X.; Wang, Q.; Guo, S. In situ Synthesis of Silver-Nanoparticles/bacterial Cellulose Composites for Slow-released Antimicrobial Wound Dressing. Carbohydr. Polym. 2014, 102, 762-771.
(17) Kelly, F. M.; Johnston, J. H. Colored and Functional Silver Nanoparticle-Wool Fiber Composites. ACS Appl. Mater. Interfaces 2011, 3, 1083-1092.
(18) Boroumand, M. N.; Montazer, M.; Simon, F.; Liesiene, J.; Saponjic, Z.; Dutschk, V. Novel Method for Synthesis of Silver Nanoparticles and their Application on Wool. Appl. Surf. Sci. 2015, 346, 477-483.
(19) Hill, P.; Brantley, H.; Van Dyke, M. Some Properties of Keratin Biomaterials: Kerateines. Biomaterials 2010, 31, 585-593.
(20) Vasconcelos, A.; Cavaco-Paulo, A. The Use of Keratin in Biomedical Applications. Curr. Drug Targets 2013, 14, 612-619.
(21) Sando, L.; Kim, M.; Colgrave, M. L.; Ramshaw, J. A.; Werkmeister, J. A.; Elvin, C. M. Photochemical Crosslinking of Soluble Wool Keratins Produces a Mechanically Stable Biomaterial that Supports Cell Adhesion and Proliferation. J. Biomed. Mater. Res., Part A 2010, 95, 901-911.
(22) Yamauchi, K.; Maniwa, M.; Mori, T. Cultivation of Fibroblast Cells on Keratin-coated Substrates. J. Biomater. Sci., Polym. Ed. 1998, 9, 259-270.
(23) Cui, L.; Gong, J.; Fan, X.; Wang, P.; Wang, Q.; Qiu, Y. Trans Glutaminase-modified Wool Keratin Film and its Potential Application in Tissue Engineering. Eng. Life Sci. 2013, 13, 149-155.
(24) Xu, S.; Sang, L.; Zhang, Y.; Wang, X.; Li, X. Biological Evaluation of Human Hair Keratin Scaffolds for Skin Wound Repair and Regeneration. Mater. Sci. Eng., C 2013, 33,648-655.
(25) de Guzman, R. C.; Merrill, M. R.; Richter, J. R.; Hamzi, R. I.; Greengauz-Roberts, O. K.; Van Dyke, M. E. Mechanical and Biological Properties of Keratose Biomaterials. Biomaterials 2011, 32, 8205-8217.
(26) Iqbal, H. M. N.; Kyazze, G.; Locke, I. C.; Tron, T.; Keshavarz, T. In situ Development of Self-defensive Antibacterial Biomaterials: Phenol-g-keratin-EC based Biocomposites with Characteristics for Biomedical Applications. Green Chem. 2015, 17, 3858-3869.
(27) Aluigi, A.; Vineis, C.; Varesano, A.; Mazzuchetti, G.; Ferrero, F.; Tonin, C. Structure and Properties of Keratin/PEO blend Nanofibers. Eur. Polym. J. 2008, 44, 2465-2475.
(28) Khosa, M. A.; Ullah, A. A Sustainable Role of Keratin Biopolymer in Green Chemistry: A Review. J. Food Proc. Beverages 2013, 1, 8-15.
(29) Rosewald, M.; Hou, F. Y. S.; Mututuvari, T.; Harkins, A. L.; Tran, C. D. Cellulose-Chitosan-Keratin Composite Materials: Synthesis and Immunological and Antibacterial Properties. ECS Trans. 2014, 64 (4), 499-505.
(30) Tran, C. D.; Mututuvari, T. Cellulose, Chitosan and Keratin Composite Materials. Controlled Drug Release. Langmuir 2015, 31, 1516-1526.
(31) Tran, C. D.; Mututuvari, T. Cellulose, Chitosan and Keratin Composite Materials. Facile and Recyclable Synthesis, Conformation and Properties. ACS Sustainable Chem. Eng. 2016, 4, 1850-1861.
(32) Tran, C. D.; Prosenc, F.; Franko, M.; Benzi, G. Synthesis, Structure and Antimicrobial Property of Green Composites from Cellulose, Wool, Hair and Chicken Feather. Carbohydr. Polym. 2016, 151, 1269-1276.
(33) Tran, C. D.; Duri, S.; Harkins, A. L. Recyclable Synthesis, Characterization and Antimicrobial Activity of Chitosan-based Polysaccharide Composite Materials. J. Biomed. Mater. Res., Part A 2013, 101, 2248-2257.
(34) Harkins, A. L.; Duri, S.; Kloth, L. C.; Tran, C. D. Chitosan-Cellulose Composite for Wound Dressing Material. Part 2. Antimicrobial Activity, Blood Absorption Ability and Biocompatibility. J. Biomed. Mater. Res., Part B 2014, 102 (6), 1199-1206.
(35) Mututuvari, T. M., Supramolecular Biopolymeric Composite Materials: Green Synthesis, Characterization and Applications. Dissertation, Marquette University, Wilwaukee, W I, 2014.
(36) Li, R.; Wang, D. Preparation of Regenerated Wool Keratin Films from Wool Keratin-ionic liquid Solutions. J. Appl. Polym. Sci. 2013, 127, 2648-2653.
(37) Peplow, P. V.; Roddick-Lanzilotta, A. D. Orthopaedic Materials Derived from Keratin. U.S. Patent 2005/0232963 A1, 2005.
(38) Fang, J. Y.; Chen, J. P.; Leu, Y. L.; Wang, H. Y. Characterization and Evaluation of Silk Protein Hydrogels for Drug Delivery. Chem. Pharm. Bull. 2006, 54, 156-162.
(39) JCPDS file No 31-1238.
(40) Dong, R.; Tian, B.; Zeng, C.; Li, T.; Wang, T.; Zhang, J. Ecofriendly Synthesis and Photocatalytic Activity of Uniform Cubic Ag@AgCl Plasmonic Photocatalyst. J. Phys. Chem. C 2013, 117, 213-220.
(41) Veronica da Silva Ferreiraa, V. D. S.; ConzFerreiraa, M. E.; Lima, L. M. T. R. Green Production of Microalgae-based Silver Chloride Nanoparticles with Antimicrobial Activity against Pathogenic Bacteria Enzym. Microbial. Tech. 2016 Ahead of print http://dx.doi.org/10.1016/j.enzmictec.2016.10.018.
(42) Dhas, T. S.; Kumar, V. G.; Karthick, V.; Angel, K. J.; Govindaraju, K. Facile Synthesis of Silver Chloride Nanoparticles using Marine Alga and its Antibacterial Efficacy, Spectrochim. Acta A Mol. Biomol. Spectrosc. Acta A Mol. Biomol. Spectrosc. 2014, 120, 416-420.
(43) Sohrabnezhad, Sh.; Rassa, M.; Dahanesari, E. M. Spectroscopic Study of Silver Halides in Montmorillonite and their Antibacterial Activity. J. Photochem. Photobiol., B 2016, 163, 150-155.
(44) Sharma, P.; Sanpui, P.; Chattopadhyay, A.; Ghosh, S. Fabrication of Antibacterial Silver Nanoparticle-Sodium Alginate-Chitosan Composite Films. RSC Adv. 2012, 2, 5837-5843.
(45) Sathishkumar, M.; Sneha, K.; Yun, Y. S. Immobilization of Silver Nanoparticles Synthesized using Curcuma Longa Tuber Powder and Extract on Cotton Cloth for Bactericidal Activity. Bioresour. Technol. 2010, 101, 7958-7965.
(46) JCPDS 04-0783.
(47) Scherrer, P. Bestimmung der Grisse und der Inneren Struktur von Kolloidteilchen Mittels Rintgenstrahlen, Nachr. Ges. Wiss. Gittingen 1918, 26, 98-100.
(48) Langford, J. J.; Wilson, A. J. C. Scherrer after Sixty Years: A Survey and Some New Results in the Determination of Crystallite Size. J. Appl. Crystallogr. 1978, 11, 102-113.
(49) Korte, D.; Concetta Bruzzoniti, M.; Sarzanini, C.; Franko, M. Thermal Lens Spectrometric Determination of Colloidal and Ionic Silver in Water. Int. J. Thermophys. 2011, 32, 818-827.
(50) Tran, C. D.; Franko, M. Thermal Lens Spectroscopy. In Encyclopedia of Analytical Chemistry; Meyers, R. A., Ed.; John Wiley: Chichester, U.K., 2010; DOI: 10.1002/9780470027318.a9079.
(51) Korte, D.; Bruzzoniti, M. C.; Sarzanini, C.; Franko, M. Thermal Lens Spectrometric Determination of Colloidal and Ionic Silver in Water. Int. J. Thermophys., 2011, 32, 818-827.
(52) Tran, C. D.; Franko, M. “Thermal Lens Spectroscopy” In: Encyclopedia of Analytical Chemistry, ed.: R. A. Meyers, John Wiley: Chichester., 2010. DOI: 10.1002/9780470027318.a9079.

### Example 3—Cellulose, Chitosan and Keratin Composite Materials: Facile and Recyclable Synthesis, Conformation and Properties

Reference is made to Tran et al., “Cellulose, Chitosan and Keratin Composite Materials: Facile and Recyclable Synthesis, Conformation and Properties,” ACS Sustainable Chem. Eng. 2016, 4, 1850-1861, the content of which is incorporated herein by reference in its entirety.

Abstract

A method was developed in which cellulose (CEL) and/or chitosan (CS) were added to keratin (KER) to enable [CEL/CS+KER] composites formed to have better mechanical strength and wider utilization. Butylmethylimmidazolium chloride ([BMIm⁺Cl⁻]), an ionic liquid, was used as the sole solvent, and because the majority of [BMIm⁺Cl⁻] used (at least 88%) was recovered, the method is green and recyclable. FTIR, XRD, ¹³C CPMAS NMR and SEM results confirm that KER, CS and CEL remain chemically intact and distributed homogeneously in the composites. We successfully demonstrate that the widely used method based on the deconvolution of the FTIR bands of amide bonds to determine secondary structure of proteins is relatively subjective as the conformation obtained is strongly dependent on the choice of parameters selected for curve fitting. A new method, based on the partial least squares regression analysis (PLSR) of the amide bands, was developed, and proven to be objective and can provide more accurate information. Results obtained with this method agree well with those by XRD, namely they indicate that although KER retains its second structure when incorporated into the [CEL+CS] composites, it has relatively lower α-helix, higher β-turn and random form compared to that of the KER in native wool. It seems that during dissolution by [BMIm⁺Cl⁻], the inter- and intramolecular forces in KER were broken thereby destroying its secondary structure. During regeneration, these interactions were reestablished to reform partially the secondary structure. However, in the presence of either CEL or CS, the chains seem to prefer the extended form thereby hindering reformation of the α-helix. Consequently, the KER in these matrices may adopt structures with lower content of α-helix and higher β-sheet. As anticipated, results of tensile strength and TGA confirm that adding CEL or CS into KER substantially increase the mechanical strength and thermal stability of the [CS/CEL+KER] composites.

Introduction

Nonantigenic keratin is known to possess advantages for wound care, tissue reconstruction, cell seeding and diffusion, and drug delivery as topical or implantable biomaterial.¹⁻⁵ As implantable film, sheet, or scaffold, keratin can be absorbed by surrounding tissue to provide structural integrity within the body while maintaining stability under mechanical load, and in time can break down to leave neo-tissue. Keratin is found to be characteristically abundant in cysteine residues (7-20% of the total amino acid residues).¹⁻⁵These cysteine residues are oxidized to give inter- and intramolecular disulfide bonds, which results in three-dimensionally linked network of keratin fiber. Interestingly, in spite of its unique structure, keratin has relatively poor mechanical properties, and as a consequence, it was not possible to exploit fully unique properties of keratin for various applications.¹⁻⁵

Polysaccharides such as cellulose (CEL) are known to have strong mechanical property,^{6,7} and chitosan (CS) to have ability to stop bleeding (hemostasis), heal wounds, kill bacteria and adsorb organic and inorganic pollutants.⁸⁻¹¹ It is, therefore, possible that adding CEL and/or CS to KER would enhance the mechanical properties of the [CEL/CS+KER] composites so that they can be practically used for a variety of applications that hitherto were not possible. We have demonstrated recently that a simple ionic liquid, butylmethylimmidazolium chloride ([BMIm⁺Cl⁻]), can dissolve both CEL and CS, and by use of this [BMIm⁺Cl⁻] as the sole solvent, we developed a simple, GREEN and totally recyclable method to synthesize [CEL+CS] composites just by dissolution without using any chemical modifications or reactions.¹²⁻¹⁴ The [CEL+CS] composite obtained was found to be not only biodegradable and biocompatible but also retain unique properties of its component.²⁻¹⁴Because [BMIm⁺Cl⁻] can also dissolve KER, it may be possible to use this IL as the sole solvent to synthesize [CEL/CS+KER] composites in a single step.

Such consideration prompted us to initiate this study that aims to improve the mechanical properties of the KER composites by adding either CEL or CS to the composites, and to demonstrate that the composites will retain unique properties of their components. In this paper, we will report results of the synthesis and spectroscopic characterization of the [CEL/CS+KER] composites. We will also report on the novel partial least squares regression (PLSR) method that we develop to determine the secondary structure of KER in the composites.

The motivation for us to develop this PLSR method stems from the fact that results from our previous studies indicate that dissolution by and regeneration from [BMIm⁺Cl⁻] do not alter chemical structure of CEL and CS.¹²⁻¹⁴ It is possible that the regenerated KER may also retain some of its structure as well. It is known that different from polysaccharides, which are known to have only random structure, the protein KER has secondary structure. The secondary structure of KER in [CEL/CS+KER] composites may be modified during the synthesis. It is of particular importance to determine how much of the secondary structure (α-helix and β-sheet) is retained when it is incorporated into the [CEL+CS+KER] in composites. Such information is important because, the secondary structure of the composites strongly affects their properties including porosity, antimicrobial and antiviral activity and their ability to encapsulate and controlled release of drugs.

Circular dichroism (CD) is known to be very effective for the determination of protein secondary structure but it is effective only for solution phase.¹⁵⁻¹⁷ When used for solid samples, particularly for amorphous solids, it is seriously plagued by many artifacts including induced linear dispersion and linear birefringence and depolarization at grain boundaries.^{16,17} Solution NMR can provide information on the location of secondary structural elements within the protein sequence.¹⁸⁻²⁰ It is, however, effective only for proteins with MW<30K and with knowledge on chemical shifts of particular residues in the protein.¹⁸⁻²⁰ Because MWs of CEL, CS and KER are much higher than 40-70 KDa, it is not possible to use NMR for the composites. As will be demonstrated in the section below, a method based on the deconvolution of the FTIR amide I band into bands corresponding to α-helix, β-sheet and random is subjective as it is strongly dependent on choice of parameters selected for curve fitting.²¹⁻²³

In this paper, we describe the theory of the novel PLSR method that we have developed, and report on experimental results obtained using this method to demonstrate clearly that it is more objective and provides accurate results than all other methods.

Experimental Methods

Chemicals.

Chitosan (MW≈310-375 kDa), and microcrystalline cellulose (DP≈300)¹²-1⁴, were purchased from Sigma-Aldrich (Milwaukee, Wis.). The degree of deacetylation of chitosan, determined by FT-IR, was found to be 84±2% 0.13 Raw sheep (untreated) wool, obtained from a local farm, was cleaned by Soxhlet extraction using a 1:1 (v/v) acetone/ethanol mixture at 80±3° C. for 48 h. The wool was then rinsed with distilled water and dried at 100±1° C. for 12 h.² 1-Methylimidazole and n-chlorobutane (Alfa Aesar, Ward Hill, Mass.) were distilled prior to using for synthesis of [BMIm⁺Cl⁻]¹²⁻¹⁴.

The protein standards, used to construct a PLSR model to estimate the secondary structure of KER, included albumins (bovine serum albumin, BSA and human serum albumin, HSA); hemoglobin (horse, HEM); lysozyme (egg white, LYZ); myoglobin (horse skeletal muscle, MYO); pepsin A (porcine stomach, PEP); ribonuclease A (bovine pancrease, RNASE A); and trypsin inhibitor (soybean, SOY). Except for PEP and SOY, which were purchased from Worthington Biochemical Corporation (Lakewood, N.J.), all the other protein standards were purchased from Sigma Aldrich (St Louis, Mo.). All the proteins were received in lyophilized powder form and they were used without further purification.

Instruments.

FTIR spectra (from 450-4,000 cm⁻¹ were recorded on a Spectrum 100 Series FTIR spectrometer (Perkin Elmer, USA) at resolution of 2 cm⁻¹ by the KBr method. Each spectrum was an average of 64 individual spectra. X-ray diffraction (XRD) measurements were taken on a Rigaku MiniFlex II diffractometer utilizing the Ni filtered Cu Kα radiation (1.54059 Å). The voltage and current of the X-ray tube were 30 kV and 15 mA respectively. The samples were measured within the 20 angle range from 2.0 to 40.00. The scan rate was 50 per minute. Data processing procedures were performed with the Jade 8 program package²⁴. The surface and cross-sectional morphologies of the composite films were examined under vacuum with a JEOL JSM-6510LV/LGS Scanning Electron Microscope with standard secondary electron (SEI) and backscatter electron (BEI) detectors. Prior to SEM examination, the film specimens were made conductive by applying a 20 nm gold-palladium-coating onto their surfaces using an Emitech K575x Peltier Cooled Sputter Coater (Emitech Products, TX). The tensile strength of the composite films were evaluated on an Instron 5500R tensile tester (Instron Corp., Canton, Mass.) equipped with a 1.0 kN load cell and operated at a crosshead speed of 5 mm min⁻¹. Each specimen had a gauge length and width of 25 mm and 10 mm respectively. Thermogravimetric analyses (TGA) (TG 209 F1, Netzsch) of the composite films were investigated at a heating rate of 10° C. min⁻¹ from 30-600° C. under a continuous flow of 20 mL min⁻¹ nitrogen gas.

Determination of Secondary Structure of Keratin by Deconvoluting Amide I Band.

Amide I band in the FTIR spectrum was deconvoluted into individual Gaussian bands using Origin Pro 9.0 software (OriginLab, USA). Each band was integrated to obtain its area. The individual bands were assigned to α-helix (1657-1650 cm⁻¹), β-sheet (1640-1612 cm⁻¹), and disordered (1697-1670 cm⁻¹) conformations.^{25,26} Then, the proportional content of each band was calculated by dividing the area of the band by the total area of all the bands within the amide I region.

Determination of Secondary Structure of Keratin by Partial Least Squares Regression (PLSR) Method.

Multivariate data analysis by PLS regression (PLSR) was carried out using Unscrambler X10.1 software (CAMO Inc., Oslo, Norway). A detailed treatment of this PLSR is described elsewhere.²⁷ Briefly, PLSR builds a linear model that relates two data matrices, the predictors (X) and the response (Y), to each other by using least squares fitting technique. In this case, X contains spectra of each of the eight protein standards from 1700 to 1450 cm⁻¹; this frequency range was chosen because it was reported to contain much information about the secondary structure of proteins.²⁸⁻³¹ Y contains information about the secondary structure of the standard proteins. The model can therefore be represented by the equation:

Y=XB (1)

where B contains columns of regression coefficients at each frequency. The goal is to calculate B which can subsequent be used to predict the composition of the unknown. In PLSR, B is calculated by decomposing X and Y matrices into latent variables (principal components, PCs) which maximize covariance between X and Y. After obtaining the B matrix, the secondary structure can then be calculated using the relation:

Y=Bx (2)

where x is the spectrum of the unknown protein sample.

The success of PLSR is determined by selecting those frequency variables which correlate well with the secondary structure motifs (i.e. α and β). The method of cross model validation (CMV) with Jack-knifing was selected for this purpose.^{32,33} This model ensures variable selection without over-fitting and or selecting false positive variables.^{34,35} Each inner model in CMV consisted of seven proteins each time. For Jack-knifing, variables with p-value less than 0.05 for either α-helix or β-sheet were considered significant and therefore retained in the model. It is noteworthy to add that all spectra were baseline corrected and autoscaled by standard normal variate (SNV) method. In addition, the data were mean centered each time before PLSR modeling. All these routines are already integrated in the Unscrambler software that was used.

The X-ray structures of the eight proteins, constituting our reference set, were taken from the Protein Data Bank (PDB).³⁶ The secondary structure of these proteins were evaluated using the algorithm Define Secondary Structure of Proteins (DSSP) which is integrated in the PDB program. The DSSP algorithm works by assigning secondary structure to the amino acids of a protein given the atomic resolution coordinates of the protein. Details on this method are presented elsewhere.³⁷ Based on this algorithm, eight types of secondary structure are assigned. However, in this study, only 3 groups were assigned viz α-helix, β-sheet and the remainder was assigned to unordered group. The proteins used together with their PDB IDs and resultant secondary structures are listed in Table SI-1 of the Supporting Information.

Results and Discussion

Synthesis of [CEL/CS+KER] Composites.

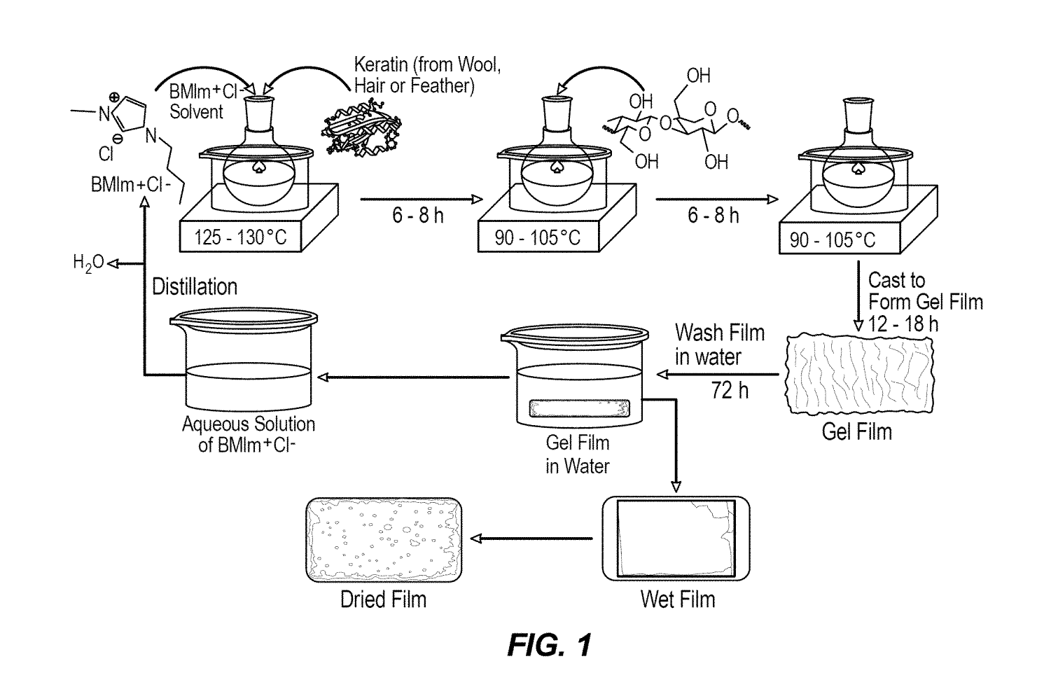

We successfully synthesized one-(CEL/CS, and KER), two- ([CEL+KER], [CEL+CS] and three- ([CEL+CS+KER]) component composite films by using [BMIm⁺Cl⁻], an ionic liquid, to dissolve CEL, CS and KER. As shown on FIG. 1, wool dissolution required relatively higher temperature (120° C.) than that needed for either CEL or CS (90° C.). This may be due to the types of bond networks present in these biopolymers. The three dimensional structures of CEL, CS and KER are known to be stabilized by inter- and intra-molecular hydrogen bonding. In addition to this hydrogen bonding network, KER has an extensive network of disulfide (—S—S—) linkages both within and between its protein chains. It seems that this additional bond network imparts additional tightness into its structure thereby impeding the penetration of solvent molecules into its fibers. As a consequence, higher temperature is needed to dissolve the wool.

When synthesizing two- or three-component films, it was found that the order of addition of the biopolymers is very critical. For example, all KER-based composites were synthesized by first dissolving wool at 120° C. Once dissolved, the solution temperature was reduced to 90° C. before CEL or CS was added to the KER solution. Initially, when CEL was added in 1% weight portions to the BMIm⁺Cl⁻ solution of KER, it seems that the latter enwrapped around the former, leading to the formation of small lumps of CEL. It was time-consuming and difficult to completely dissolve these lumps. To circumvent this problem, a smaller amount (ca 0.5% weight portion) of CEL was subsequently added. For the synthesis of [CEL+CS] composites, CEL was dissolved first before adding CS. If CS is dissolved first, it would form relatively high viscous solution which would make it difficult to completely dissolve CEL, which may produce inhomogeneous composite material. Using this procedure, [BMIm⁺Cl⁻] solution of CEL, CS and KER containing up to total concentration of 6 wt % (relative to IL) with various compositions and concentrations were prepared.

The resulted solution was cast onto PTFE moulds with desired thickness on Mylar films to produce thin films of 2- and 3-component films with different compositions and concentrations of CEL, CS and KER. They were then allowed to undergo gelation at room temperature to yield gel films. Because [BMIm⁺Cl⁻] is known to exhibit some toxicity to living organisms¹²⁻¹⁴ it was removed from the composites by washing the gel films with water for at least three days. The washing water was replaced with fresh water 3 times on the first day and 2 times on day 2 and day 3. Concentration of [BMIm⁺Cl⁻] in the wash water was determined by UV-visible absorption at 209 nm. Based on the absorptivity of [BMIm⁺Cl⁻], at the end of the washing, if any of the IL was present in the wash water, it was less than 56 pg/1 mL of water. Since no [BMIm⁺Cl⁻] was detected on the composite films by FTIR, NIR and UV, it is very likely that the IL was completely removed from the films by washing them with water. Even if any of it ever remained, it would be of less than 56 pg/1 g of composite film. The [BMIm⁺Cl⁻] in wash water was recovered by distilling the wash solution, and then dried under vacuum at 70° C. overnight before being reused. Finally, dried films were obtained when the wet films were allowed to dry at room temperature in a humidity-controlled chamber.

Spectroscopic Characterization.

Fourier Transform Infrared (FTIR was used to 1) confirm that CEL, CS and KER were not chemically altered by dissolution with and regeneration from ionic liquids; and 2) determine the secondary structure of keratin the [CEL+CS+KER] composite films.

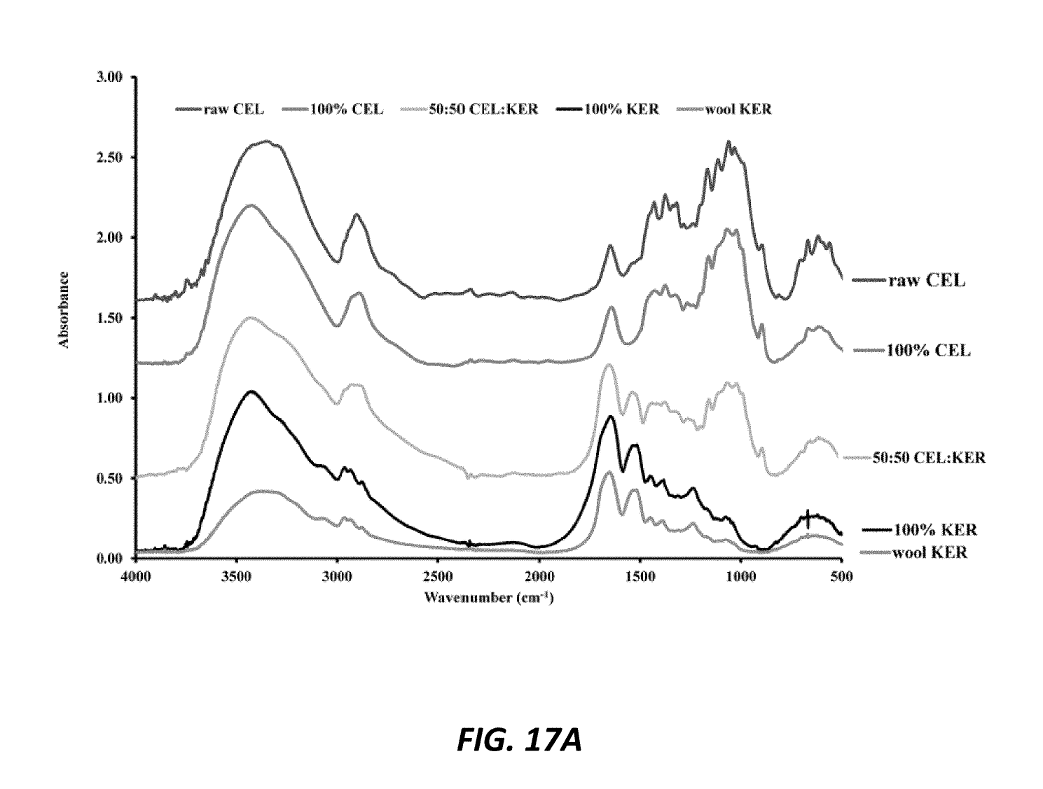

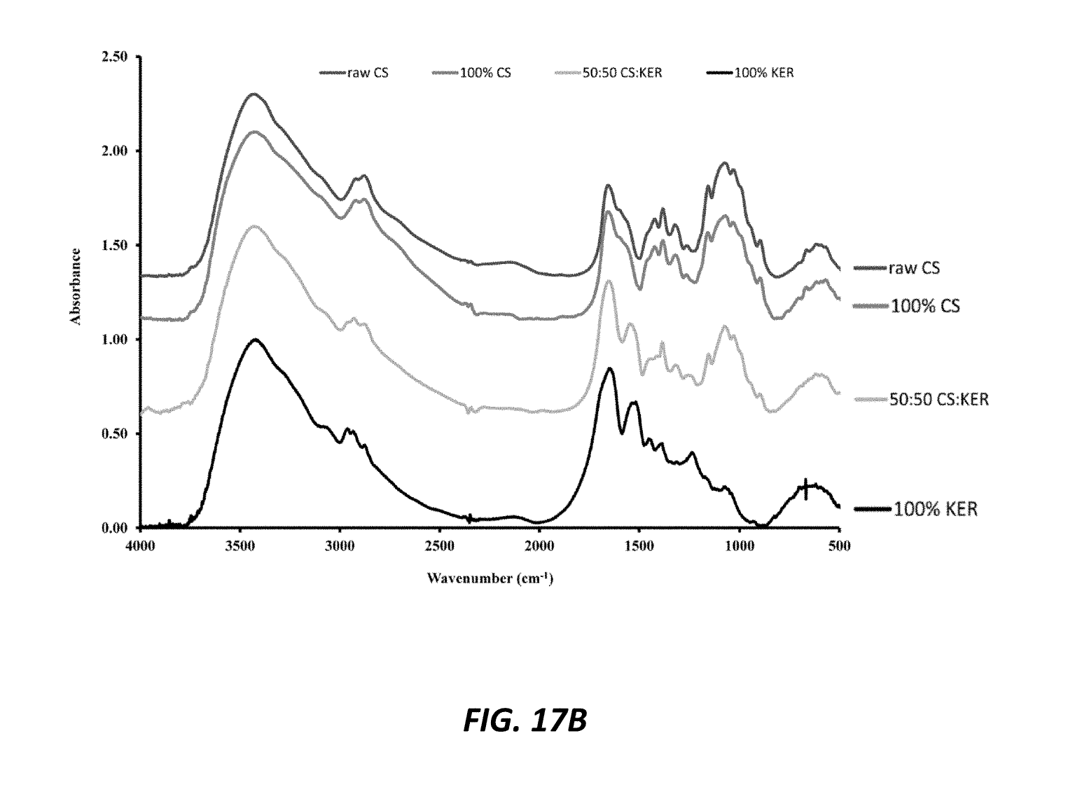

FTIR spectra of wool, shown as the pink curve in FIGS. 17A and B, exhibited characteristic bands that can be assigned to the vibrational modes of peptide bonds in proteins. For examples; the bands at 1700-1600 cm⁻¹ and 1550 cm⁻¹ are due to amide C═O stretch (amide I) and C—N stretch (amide II) vibrations respectively³⁸. In addition, the 3280 cm⁻¹ band can be assigned to N—H stretch vibration (amide A) whilst a band at 1300-1200 cm⁻¹ is due to the in-phase combination of the N—H bending and the C—N stretch vibrations (amide III). This finding is expected since wool contains more than 95% of keratin protein.³⁹ It is noteworthy to add that the FTIR spectrum of wool does not have any band at 1745 cm⁻¹, which is known to be due to lipid ester carbonyl vibrations.⁴⁰ It seems, therefore, that the Soxhlet extraction effectively removed all residual lipids from wool. Interestingly, upon regenerating KER film from the wool, no new IR signatures were detected in the FTIR spectrum of the former (compare pink spectrum for wool to the black spectrum for 100% KER). This suggests that dissolution by and regeneration of KER from BMIm⁺Cl⁻ do not produce any chemical alteration on the chemical structure of KER. It is, therefore, reasonable to expect that the properties of wool may remain intact in the regenerated KER film.

The FTIR spectra of [CEL+KER] and [CS+KER] composites with different compositions are presented in FIGS. 17 (A) and (B). As expected, the spectra of these composite films exhibit bands characteristic of their respective components. Furthermore, the magnitude of these bands seems to correlate well with concentration of corresponding component in the film. For example; the band between 1200- and 900-cm⁻¹ (due to sugar ring deformations) increased in relative intensity concomitantly with the relative concentration of CEL in the [CEL+KER] composite films (FIG. 17A). On the other hand, the intensity of the amide I and amide II bands increased with the increase in the relative concentration of KER in the same composite films. Similar behavior was also observed for [CS+KER] composite films (FIG. 17B). It is noteworthy to add that, in all composite films ([CEL+KER], [CS+KER] and [CEL+KER+CS]), no new bands are found in their FTIR spectra; i.e., the spectra of the composites are a superposition of the spectra of the corresponding individual components. This, as noted earlier, further confirms that no chemical alterations occurred during the synthesis of these composites, and that the composites obtained are expected to retain the properties of their components.

Analysis of Secondary Structure of Keratin and its Composites.

As stated above, the main chemical framework of KER was maintained during the regeneration process. It is, however, possible that its secondary structure was modified during the process. Such changes may adversely affect the properties of KER. It is, therefore, essential to determine the secondary structure of regenerated KER.

As described in the introduction, method such as circular dichroism (CD) and NMR are not suited for the [CEL/CS+KER] composites because in addition to being amorphous, the molecular weight of the composites is too high for the NMR method to be effective.

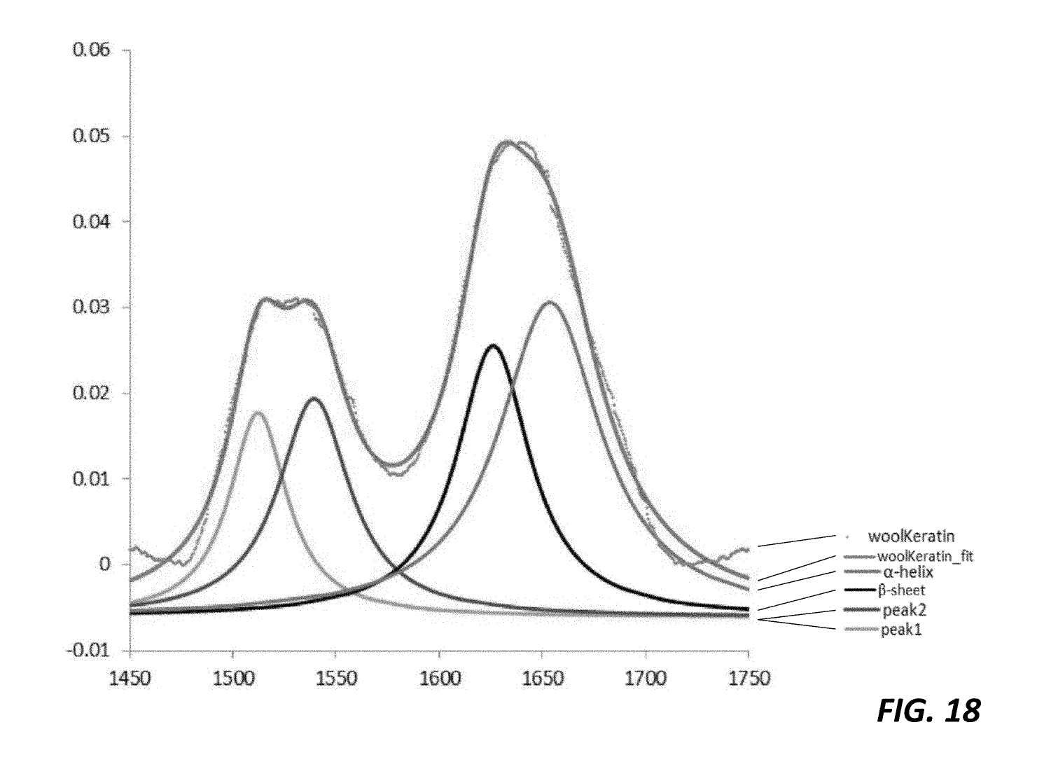

The FTIR method is based on the deconvolution of the FTIR amide I band into underlying bands which are assigned to α-helix, β-sheet and random form of a protein. Shown in FIG. 18 are results obtained by deconvoluting the amide band of the wool keratin from 1450 to 1750 cm⁻¹ into three Gaussian bands which can then be assigned to α-helix, β-sheet and random form. As illustrated, the calculated spectrum (red curve) agrees well with actual spectrum (blue dashed-line curve). Calculated concentrations of α-helix, β-sheet and random form are listed in Table 1. For reference, results for calculation made by changing the amide spectrum region by 1 or 2 cm⁻¹ in either directions are also listed in the Table. It is evidently clear that the results are very sensitive to the spectrum region selected for calculation. For example, by changing the spectrum region by only 1 cm⁻¹, i.e., from 1450-1750 cm⁻¹ to 1451-1751 cm⁻¹, the α-helix content of wool keratin increases from 45.4% to 52.2% or 15% change whereas the content of β-turn decreases from 20.6% to 14.2% or 31% change. Similarly, the content of α-helix and β-sheet for regenerated KER also increases by 5.5% and decreases by 23.4%, respectively by increasing the spectrum region used in calculation by only 1 cm⁻¹. Change of similar magnitude was also observed when the calculated spectrum region was decrease by 1 cm⁻¹; i.e., from 1450-1750 cm⁻¹ to 1449-1749 cm⁻¹.

It is, thus, clear that the deconvolution method is subjective as the conformation obtained is strongly dependent on the choice of parameters selected for curve fitting. As a consequence, our efforts were subsequently concentrated on developing a new method which is more objective so that the conformation results obtained would be more accurate and reliable. Such considerations prompted us to explore the use of the Partial Least Squares regression (PLSR) method for this purpose. In this method, only two structural motifs, α-helix and β-sheet, were modeled in the calculation even though the structure of proteins is known to be composed of varying proportions of these two motifs and other motifs (random coil or unordered). This is because the FTIR bands corresponding to α-helix and β-sheet are known to be more defined than the spectra linked to the random coil. Furthermore, the spectra linked to random coil vary from protein to protein making it difficult to accurately model this motif. Therefore, the remaining fraction, that is the fraction not attributed to any of α-helix and β-sheet, was assumed to be associated with random structures.

The first stage is to select a set of predictor (X) variables which correlate well with the response (Y) variables under study. In this case, we used cross model validation (CMV) with Jack-knifing to estimate p-values for each X-variable as described above.⁴¹ Only X-variables with p-values less than 0.05 on either α or β were retained in the model. The number of times each X-variable that was found to be significant in the eight inner models of CMV was then recorded (data not shown). A set with variables exhibiting the highest frequency (that is eight in the current case) was used to build another model. To this set, a set which exhibits the next highest frequency (that is seven) was added. This was continued until all the X-variables with frequency of at least one were used to construct the PLS model. Then, the quality of these models were evaluated based on root mean square error (RMSE), coefficient of determination (R²) and the optimal number of latent variables (LVs). The model with the lowest RMSE, highest R² and optimal number of LVs was selected for use in predicting the secondary structure of KER in wool, regenerated KER, CS:KER and CEL:KER composites. The best model that fulfilled these criteria consisted of X-variables with a frequency of significance of at least seven.

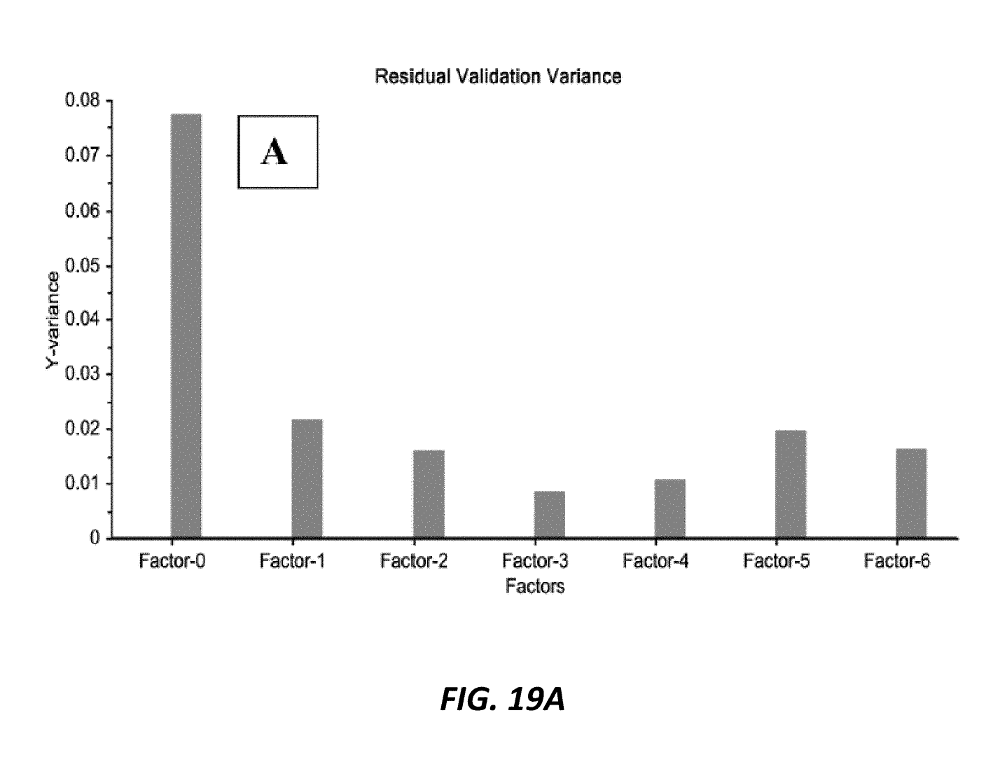

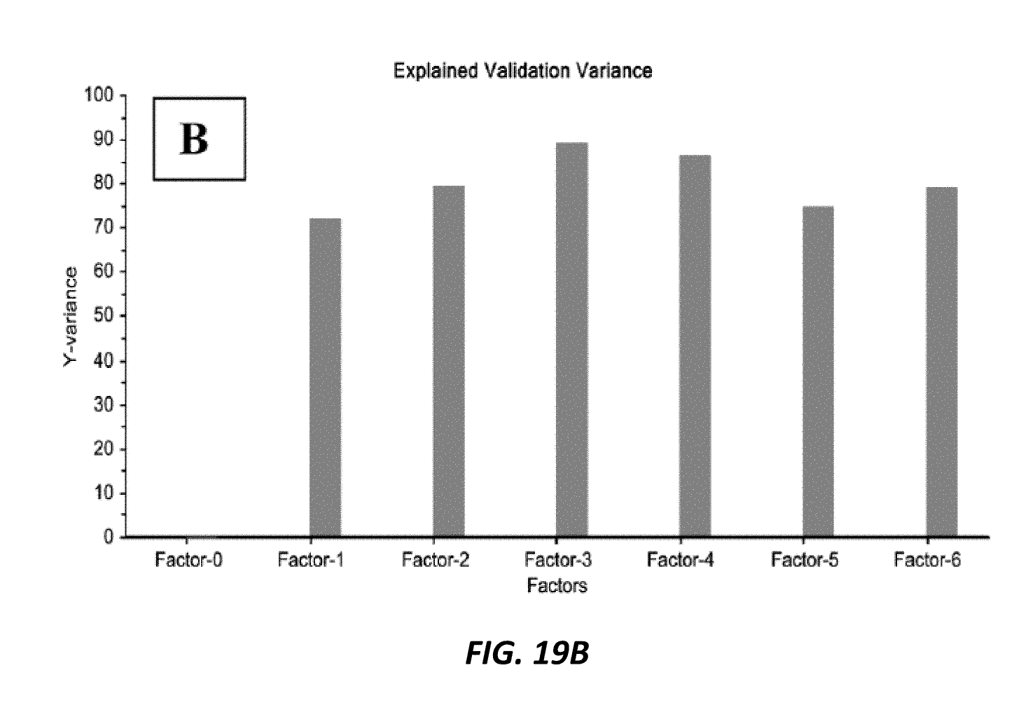

FIG. 19 summarizes the PLSR results for the chosen PLSR model. The residual validation variance tends to decrease with more factors being incorporated into the model (FIG. 19A). This is because incorporating more factors into the model produces more systematic variations. However, the residual validation variance started increasing beyond three factors, which seems to indicate that the model is now incorporating noise. Since only factors describing systematic variation should be used in the model, only three factors were used to build the calibration model for predictions of unknowns. It was also necessary to check the relative amount of variation explained when this optimum number of factors was used. FIG. 19B shows that the three factors accounted for 89% variance which is a high value. A scores plot was prepared and showed that PC1 is able to separate the protein standards based on their α-helix and β-sheet composition (data not shown). Along this PC, protein standards with more than 0.3 α-helix and at most 0.1 β-sheet (i.e. MYO, HEM, HAS, BSA, LYZ) group together, while those standards with less than 0.2 α-helix and more than 0.3 β-sheet (RNASE A, SOY, PEP) group together. These differences were further confirmed by generating a correlation loadings plot (data not shown). Along PC1, α-variable tends to appear on the right side of the plot while β-variable appears on the opposite side of the plot. By comparing this correlation loadings plot with the scores plot, it becomes apparent that α-variable is positively correlated with proteins containing more α-helix. Similarly, β-variable is positively correlated with proteins containing more β-sheet. In addition, the correlation loadings shows the correlation between the Y-variables (α and β) and the X-variables (frequency). As expected, α-helix is positively correlated to the variable 1656.5 cm⁻¹ which is consistent with the previous findings.^{17,19,22,42} On the other hand, β-sheet is positively correlated to variables 1642.0-1640.5 cm⁻¹. Plots of predicted versus reference for α-helix and β-sheet components also were generated (data not shown). Cross validation for α-helix gave RMSECV and R-square of 0.118 and 0.874 respectively whilst 0.053 and 0.934 were obtained for β-sheet. The results seems to indicate that the model predicts β-sheet content relatively more accurately than α-helix content.

The model was then applied to predict the α-helix and β-sheet contents of KER in wool, regenerated KER, CS:KER and CEL:KER composites. Results obtained are listed in Table 2. Wool was found to contain (33±2)% α-helix and (18.1±0.4)% β-sheet. These results corroborate the previous findings that sheep wool contains more α-helix than β-sheet. Upon dissolving in IL and regenerating from water, KER was found to contain (31±8)% α-helix and (21±3)% β-sheet. These results suggest that the regenerated KER adopts a similar conformation as that of wool but with relatively lower amount of (α-helix and higher β-sheet structure. Using the FTIR method based on the deconvolution of the amide band, other groups also found similar results, namely, regenerating KER leads to lower content of (α-helix and higher β-sheet structure.⁴⁴⁻⁴⁷ Subsequently, efforts were made to predict the secondary structure of KER in the CS:KER and CEL:KER composites. It is noted that the FTIR spectra of CEL and CS possess interfering bands in the amide I region. For examples, the spectrum of CEL exhibits an O—H band at 1640 cm⁻¹. Chitosan, being partially deacetylated (84±2% degree of deacetylation), contains residual amide bonds which is similar to the amide I bands. As a consequence, it is relatively more difficult to predict the secondary structure of KER composites containing either CEL or CS. However, it is expected that the interference by CS and CEL may be smaller when KER is present in relatively higher concentration. Accordingly, prediction was performed for composites containing 75% KER, namely, the 25:75 CS:KER and 25:75 CEL:KER. As shown in Table 2, the 25:75 CS:KER was found to contain (18±4)% α-helix and (31±4)% β-sheet whilst its CEL:KER counterpart contains (32±9)% α-helix and (25±4)% β-sheet. These results seem to indicate that the polysaccharides tend to stabilize more β sheet—than α helix—conformation. These results may be explained by considering the whole process of dissolution and regeneration. During dissolution, the inter- and intra-molecular forces in KER are broken thereby destroying its secondary structure but maintaining its primary structure. During gelation, regeneration from water and drying, these interactions are reestablished thereby reforming some of the same secondary structure as in wool. However, in the presence of the polysaccharides (either CEL or CS), the chains seem to prefer the extended form thereby hindering reformation of the α-helix. Consequently, the KER in these composites adopts structures with relatively lower α-helix content and higher β-sheet content.

Powder X-Ray Diffraction (XRD).

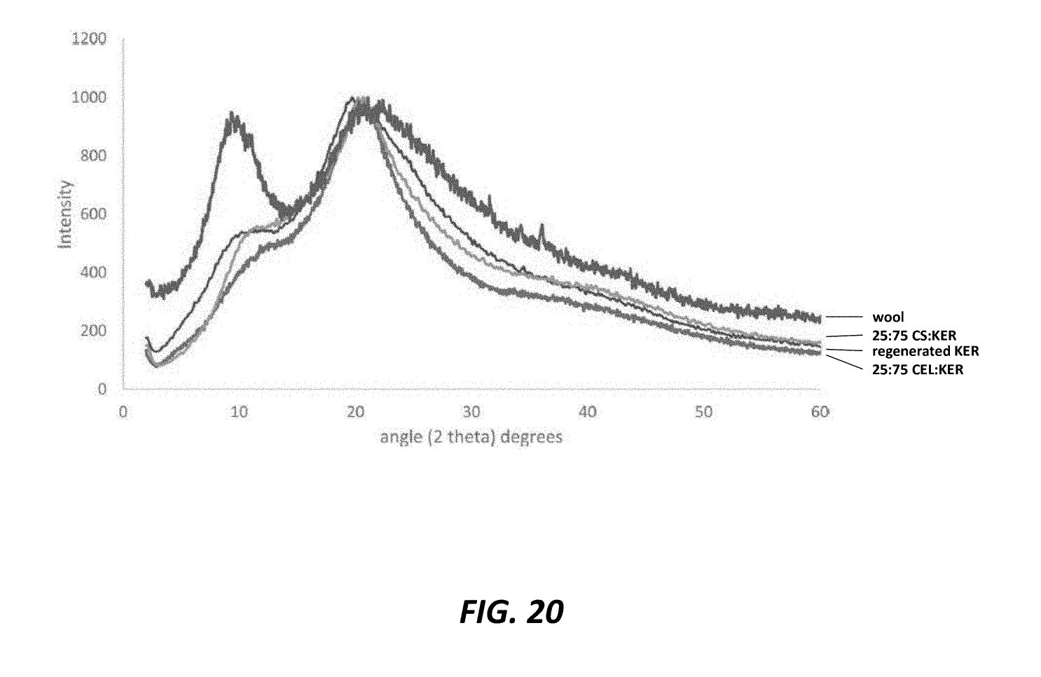

FIG. 20 shows XRD spectra for wool, regenerated KER (100% KER), 25:75 CS:KER and 25:75 CEL:KER films. Wool (red curve) exhibits two bands at 20 of about 9° and 20°. The first and the second band can be attributed to the α-helix and β-sheet structure, respectively.^{46,47} The fact that the band at ˜20° for the regenerated KER (purple curve) has the same intensity as that of the wool, but at ˜9° it has only a broad shoulder instead of a pronounced band as in wool seems to indicate that regenerated KER has relatively lower α-helix contain and higher β-sheet, β-turn and random structure than wool. Similarly, the structure of two KER composites (25:75 CS:KER and 25:75 CEL:KER (green and blue curve)) is more similar to regenerated KER than wool, namely, relatively lower α-helix and higher β-sheet, β-turn and random structure. These results are in agreement with those presented above based on FTIR, namely, it seems that during the dissolution, the inter- and intra-molecular forces in KER were broken thereby destroying its secondary structure while maintaining its primary structure. During gelation, regeneration from water and drying, these interactions are reestablished thereby partially reforming the same secondary structure as in wool. However, in the presence of the polysaccharides (either CEL or CS), the chains are maintained in the extended form thereby hindering a significant reformation of the α-helix. Consequently, the KER in these matrices may adopt structures with lower content of content of α-helix and higher β-sheet.

¹³C Solid State-Cross Polarization-Magic Angle Spinning (CP-MAS) NMR Spectroscopy.

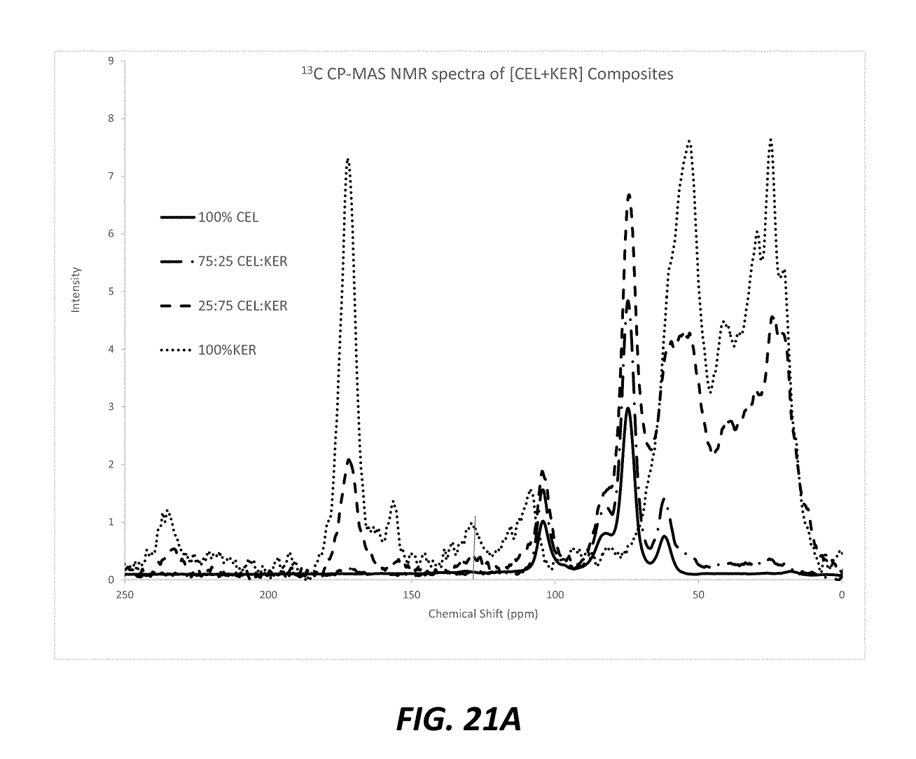

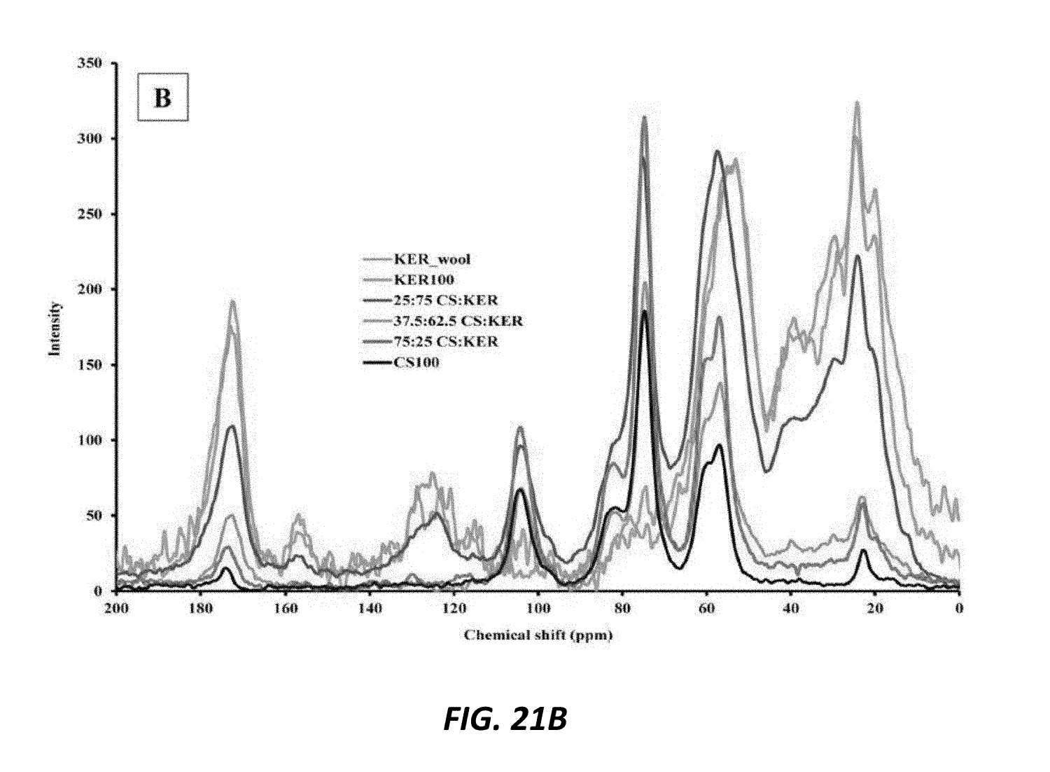

¹³C CP MAS NMR technique was used to further characterize the composites. For wool (red spectrum in FIG. 21A), the bands appearing in the ranges 172-180 ppm, 115-158 ppm, 45-65 ppm and 10-40 ppm can be assigned to carbonyl, aromatic carbons, C^{α} methane and side chain aliphatic carbon atoms respectively.^{48,49} As shown as the purple spectrum in the figure, the regenerated KER has virtually the same spectrum as that of the wool which again confirms that no chemical alteration occur during the dissolution of wool by IL and regenerating from water. The spectrum for 100% CEL film (black spectrum) contains all the bands assignable to each carbon atom of its glucose units. Specifically, the peaks appeared at 61.9 ppm (C-6), 74.6 ppm (C-3 and C-5), 83.2 ppm (C-4), 104.2 ppm (C-1). As expected, spectrum of the 25:75 CEL:KER composite (green spectrum) contains bands assignable to both CEL or KER. Similarly, the spectra of 25:75 CS:KER and 37.5:62.5 CS:KER composites contain bands corresponding to both CS and KER (FIG. 21B).

Scanning Electron Microscope (SEM).

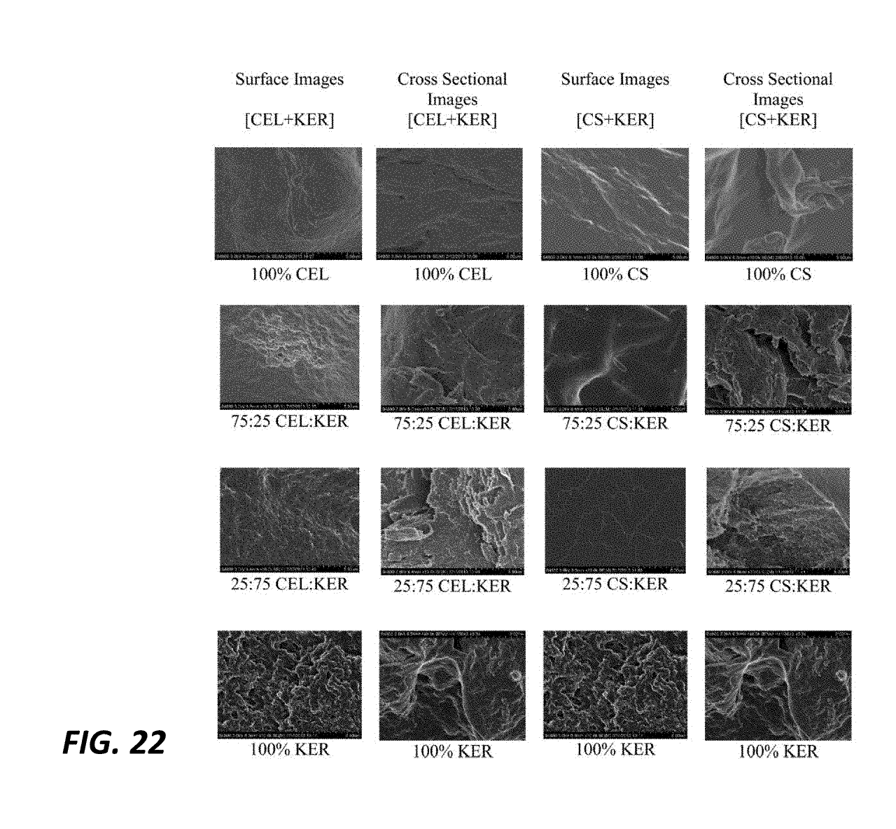

FIG. 22 shows SEM images of the surfaces and cross sections of [CEL/CS+KER] composite films. While images for 100% CS and 100% CEL surfaces exhibit smooth and homogeneous morphologies without any pores, the images of 100% KER exhibit a rough and porous structure with a three dimensional interconnection throughout the film surface. This porous structure seems to reflect the physical properties of KER films. For example, the brittleness of 100% KER film may be partly attributed to this porous microstructure. To improve the mechanical properties of KER whilst harnessing its controlled drug-release properties, KER was blended with either CEL or CS. As can be seen, incorporation of the polysaccharides (CEL and CS) into KER matrix lead to significant changes in the microstructures of the resultant composite films. However, the microstructures of these composite films are noticeably different. While incorporation of CS in the KER matrix results in composite films which present smooth and homogeneous surfaces with no evidence for phase separation, incorporation of CEL results in somewhat rough surfaces. This suggests that KER is more compatible with CS than it is with CEL. This is so despite the similarity in the chemical structures of CEL and CS; the only difference in their chemical structures is that CS has an amine group at C-2 whilst CEL has a hydroxyl group. These results seem to indicate that [CS+KER], being more densely packed, than [CEL+KER].

Mechanical Properties.

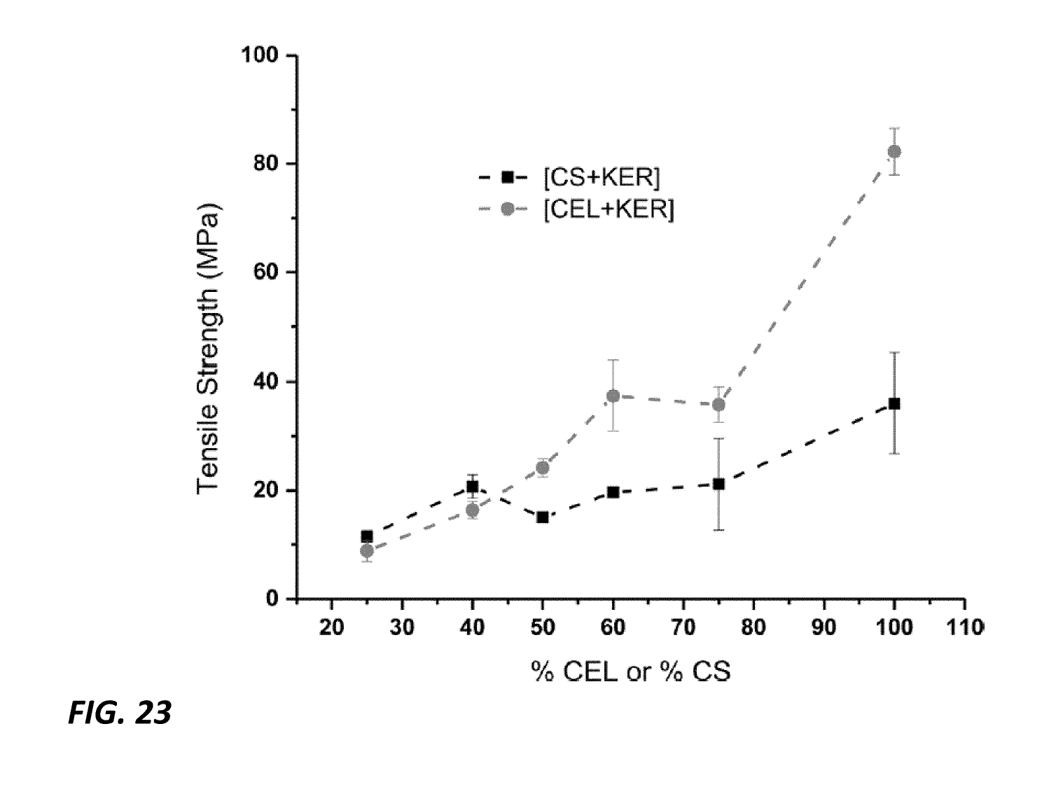

Although KER has been shown to induce controlled release of drugs,⁵⁰ its poor mechanical properties continue to restrict its potential applications. For example, as previously reported and also observed in this study,⁵⁰ regenerated KER film was found to be too brittle to be reasonably used in any application. Since CEL is known to possess superior mechanical strength, it is possible enhance the mechanical property of KER-based composite by adding CEL or other polysaccharides such as CS into it. Accordingly, [KER+CEL] and [KER+CS] composites with different concentrations were prepared, and their tensile strength was measured. FIG. 23 plots tensile strength of [CEL+KER] and [CS+KER] composites as a function of cellulose and chitosan content. As illustrated, the tensile strength of [CEL+KER] composites was found to increase concomitantly with the content of CEL. For example, the tensile strength of [CEL+KER] increased by at least 4× when CEL loading was increased from 25% to 75%. This behavior has also been reported elsewhere when CEL was used as a reinforcement in other composites.⁵¹ It is worth noting that [CEL+KER] composite films were much weaker than [CS+CEL].⁵² For example, [CEL+KER] and [CEL+CS] containing 75% and 71% CEL had tensile strengths (36±3) MPa and 52 MPa respectively. This could be attributed to the fact that CEL structure is more similar to that of CS than KER structure. Therefore much stronger interactions are established between CEL and CS than between CEL and KER. Although CS also leads to an increase in the tensile strength of [CS+KER], its effect is noticeably weaker than that of CEL of comparable loading. For example, [CEL+KER] and [CS+KER] had tensile strength values of (37±6) MPa and (20±1) MPa respectively for a 40% KER loading. Similar results were also found for 100% CS and 100% CEL, namely the tensile strengths of 100% CS is only (36±9 MPa) whereas that of 100% CEL is (82±4 MPa). The fact that CS has relatively inferior mechanical strength to CEL may be explained by the differences in the structure of CS and CEL. It is a well-known fact that the strong inter- and intramolecular hydrogen bond network in CEL enables it to adopt a strong and very dense structure thereby giving it strong mechanical strength. Compared to the hydroxy group, the amino group can only form relatively weaker hydrogen bond. The hydrogen bond network in CS is, therefore, not as extensive as in CEL, and its interior is less dense than CEL. As a consequence, CS has relatively weaker mechanical strength than CEL.

Thermal Physical Properties of [CEL/CS+KER] Composite Films.

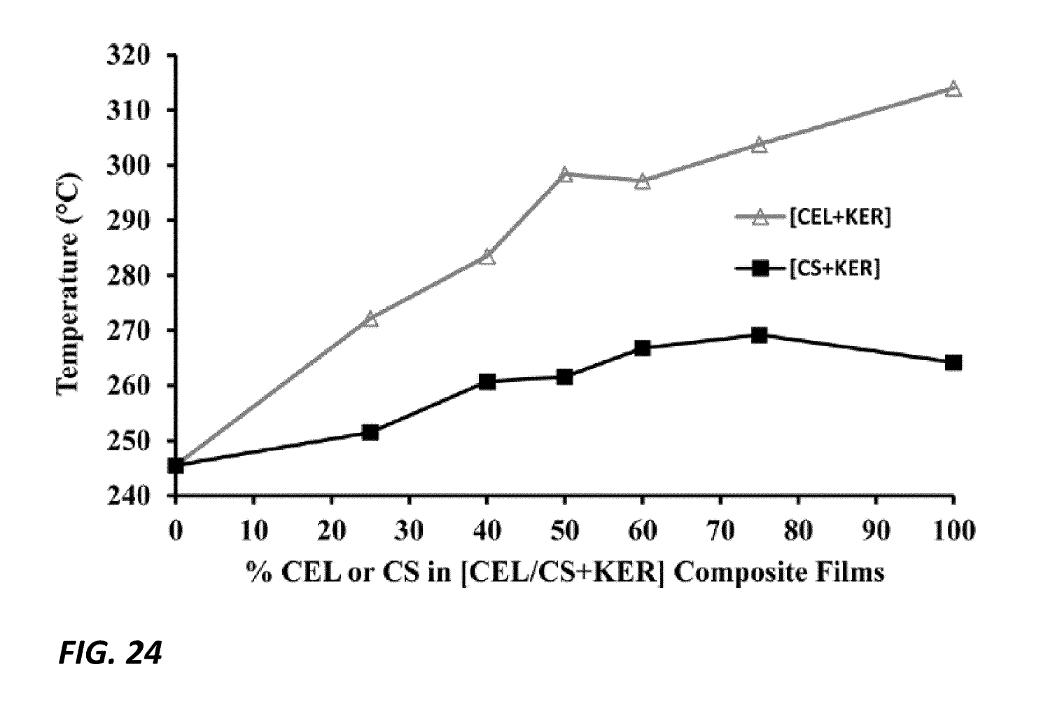

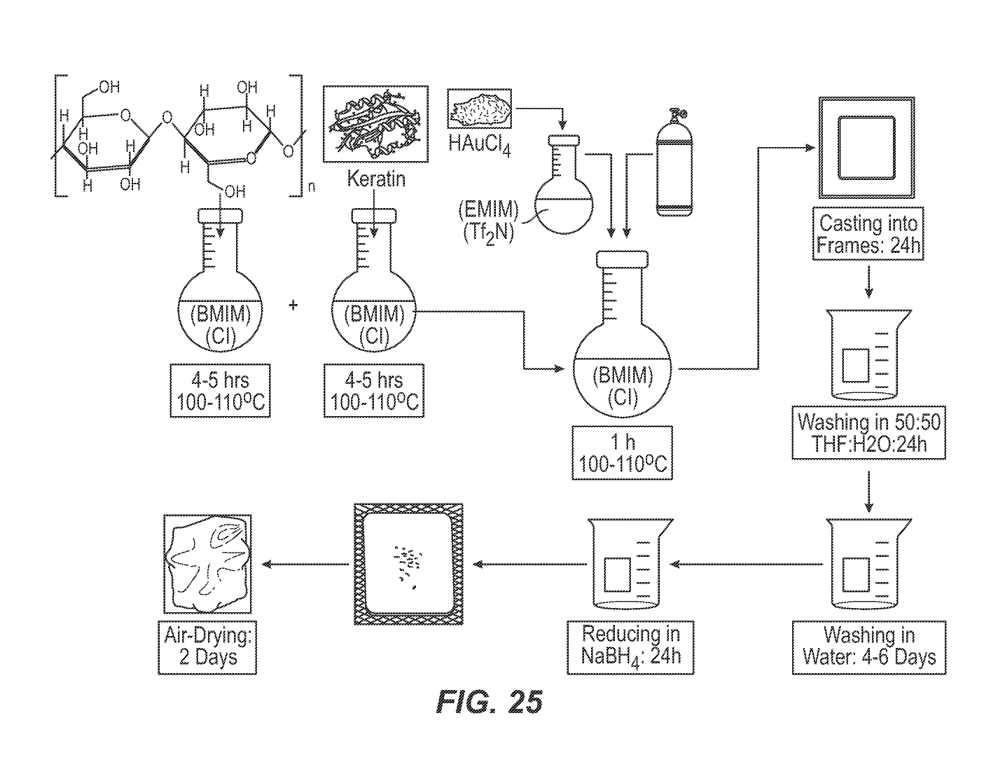

Subsequently, the thermal gravimetric analysis (TGA) was used to determine the effect of each component on the thermal properties of the resultant composite film. Effect of dissolution by IL on the thermal properties of the composites was also investigation. The comparison were achieved by using onset decomposition temperature as a surrogate measure of the thermal stability of a component. It is probable that the dissolution process could reduce the thermal stability of the biopolymers. This possibility was investigated by comparing the onset decomposition temperature of unprocessed biopolymers with corresponding regenerated films. TGA curves of wool, CEL powder, CS powder, regenerated KER, regenerated CEL (i.e., 100% CEL), regenerated CS (100% CS), and CEL:KER and CS:KER composites with different compositions were analyzed (data not shown). Also shown in the figure are derivatives of the TGA curves of these composites from which the onset decomposition temperatures of these composites were determined. It was found that the onset decomposition temperature for KER decreased by 0.5% (i.e., from 246.8 to 245.5° C.) when regenerated from IL. Similarly, the onset decomposition temperature for CS powder decreased by 2% (from 269.9 to 264.2° C.) while that of the CEL powder is by 1.26% (from 318.4 to 314.0° C.). Since these changes are small and within experimental errors, the regeneration from IL leads to only a very minor changes, if any, in the structure and thermal property of the biopolymers. These results seem to indicate that it is possible to use the TGA technique to determine the effect of adding one biopolymer to another. FIG. 24 plots onset decomposition temperature of [CS+KER] and [CEL+KER] composites as a function of concentration of CS or CEL. As illustrated, 100% KER was the least thermally stable followed by CS and then CEL. As expected, composites of KER with each of the polysaccharides show an improvement in the thermal stability as the proportional content of either CEL or CS increases. Therefore, by judiciously selecting the composition of CEL, CS and KER, the thermal properties of the [CEL/CS+KER] composites can be appropriately adjusted.

Conclusions

In summary, we have shown that KER and its composites with CEL and/or CS were successfully and readily synthesized in a one-step process in which [BMIm⁺Cl⁻], an ionic liquid, was used as the sole solvent for dissolution of the wool and polysaccharides. Since majority of [BMIm⁺Cl⁻] used (at least 88%) was recovered, the method is green and recyclable. Results of FTIR, XRD, ¹³C CP MAS NMR and SEM confirm that KER, CS and CEL remain chemically intact and homogeneously distributed in the regenerated composites. We have shown that the widely-used method based on the deconvolution of the FTIR bands of amide bonds to determine secondary structure of proteins is relatively subjective as the conformation obtained is strongly dependent on the choice of parameters selected for curve fitting. A new method, based on the Partial Least Squares Regression (PLSR) Analysis of the FTIR amide bands, was developed and proven to be objective and can provide relatively more accurate information. Results obtained with this PLSR method agree well with those deduced by XRD. Both of these methods indicate that the secondary structure of the regenerated KER and [CEL/CS+KER] composites have relatively lower α-helix, higher β-turn and random form compared to that of the KER in native wool. It seems that during dissolution by BMIm⁺Cl⁻, the inter- and intra-molecular forces in KER are broken thereby destroying its secondary structure but maintaining its primary structure. During gelation, regeneration from water and drying, these interactions are reestablished thereby reforming some of the same secondary structure as in wool. However, in the presence of the polysaccharides (either CEL or CS), the chains seem to prefer the extended form thereby hindering reformation of the α-helix. Consequently, the KER in these matrices adopts structures with lower content of α-helix and higher β-sheet. As anticipated, results of tensile strength and TGA confirm that adding CEL or CS into KER substantially increase the mechanical strength and thermal stability of the [CS/CEL+KER] composites. Since KER, CS and CEL remain chemically intact in the composites, it is expected that the composites will retain unique properties of their components. The improved mechanical and thermal physical properties of the KER composites make it possible to fully exploit its properties in various applications including antibacterial activity and drug delivery. These are the subject of our subsequent publications. In the foregoing description, it will be readily apparent to one skilled in the art that varying substitutions and modifications may be made to the invention disclosed herein without departing from the scope and spirit of the invention.

TABLE 1
Secondary structure of wool, regenerated KER and its composites
with CEL and CS calculated by deconvolution of FTIR spectra

| | Spectrum | α-helix | β-sheet | Random |
|---|---|---|---|---|
| Substance | range, cm⁻¹ | (%) | (%) | coil |
| Wool, this work | 1450-1750 | 45.4 | 20.6 | 34.1 |
| Wool, this work | 1451-1751 | 52.2 | 13.2 | 33.6 |
| Wool, this work | 1452-1752 | 51.9 | 13.9 | 34.2 |
| Wool, this work | 1449-1749 | 54.1 | 15.3 | 30.6 |
| Wool, this work | 1448-1748 | 54.7 | 15.7 | 29.6 |
| Wool, Ref 1 | 1450-1750 | 34 | 25 | |
| Wool, Ref 2 | 1580-1740 | 47 | 33 | 19 |
| Wool, Ref 3 | 1450-1750 | 58.2 | 37.9 | 3.9 |
| 100% KER | 1450-1750 | 54.4 | 7.9 | 37.8 |
| 100% KER | 1451-1751 | 57.4 | 6.0 | 36.6 |
| 100% KER | 1452-1752 | 58.9 | 6.7 | 34.4 |
| 100% KER | 1449-1749 | 59.8 | 7.4 | 32.8 |
| 100% KER | 1448-1748 | 61.1 | 8.6 | 30.3 |

TABLE 2
Secondary structure of wool, regenerated KER and its composites
with CEL and CS calculated by PLSR method

| | α-helix (%) | β-sheet (%) |
|---|---|---|
| Wool | 33 ± 2 | 18.1 ± 0.4 |
| KER100 | 31 ± 8 | 21 ± 3 |
| 25:75 CS:KER | 18 ± 4 | 31 ± 4 |
| 25:75 CEL:KER | 32 ± 9 | 25 ± 4 |

### REFERENCES

(1) Vasconcelos, A.; Cavaco-Paulo, A. The use of keratin in biomedical applications. Curr. Drug Targets 2013, 14, 612-619.
(2) Cui, L.; Gong, J.; Fan, X.; Wang, P.; Wang, Q.; Qiu, Y. Transglutaminase-modified wool keratin film and its potential application in tissue engineering. Eng. Life Sci. 2013, 13, 149-155.
(3) Xu, S.; Sang, L.; Zhang, Y.; Wang, X.; Li, X. Biological evaluation of human hair keratin scaffolds for skin wound repair and regeneration. Mater. Sci. and Eng. C 2013, 33, 648-655.
(4) de Guzman, R. C.; Merrill, M. R.; Richter, J. R.; Hamzi, R. I.; Greengauz-Roberts, O. K.; Van Dyke, M. E. Mechanical and biological properties of keratose biomaterials. Biomaterials 2011, 32, 8205-8217.
(5) Rouse, J. G.; Van Dyke, S. M.; A review of keratin-based biomaterials for biomedical applications. Materials 2010, 3, 999-1014.
(6) Augustine, A. V; Hudson, A. B; Cuculo, J. A. “Direct solvents for cellulose”, in Cellulose Sources and Exploitation; Kennedy, John F.; Phillips, Glyn O.; Williams, Peter A. ed., Ellis Horwood: New York, 1990, pp 59-65
(7) Dawsey, T. R. “Applications and limitations of lithium chloride/N,N-dimethylacetamide in the homogeneous derivatization of cellulose” in Cellulosic Polymers, Blends and Composites, Gilbert, Richard D. ed., Carl Hanser Verlag: New York, 1994, 157-171.
(8) Burkatovskaya, M.; Tegos, G. P.; Swietlik, E.; Demidova, T. N.: P. Castano, A. P.; Hamblin, M. R. Use of chitosan bandage to prevent fatal infections developing from highly contaminated wounds in mice, Biomaterials 2006, 27, 4157-4164.
(9) T. Kiyozumi, Y. Kanatani, M. Ishihara, D. Saitoh, J. Shimizu, H. Yura, S. Suzuki, Y. Okada and M. Kikuchi, Medium (DMEM/F12)-containing chitosan hydrogel as adhesive and dressing in autologous skin grafts and accelerator in the healing process, J. Biomed. Mat. Res. B: 2006, 79B, 129-136.
(10) JainD.; Banerjee, R. Comparison of ciprofloxacin hydrochloride-loaded protein, lipid, and chitosan nanoparticles for drug delivery, J. Biomed. Mat. Res. B 2008, 86, 105-112.
(11) Naficy, S.; Razal, J. M.; Spinks, G>M.; Wallace, G. G. Modulated release of dexamethasone from chitosan-carbon nanotube films, Sensors Attuators A 2009, 155, 120-124.
(12) Tran, C. D.; Duri, S.; Harkins, A. L. Recyclable synthesis, characterization, and antimicrobial activity of chitosan-based polysaccharide composite materials. J. Biomed. Mater. Res. A 2013, 101, 2248-2257.
(13) Tran, C. D.; Duri, S.; Delneri, A.; Franko, M. Chitosan-cellulose composite materials: preparation, characterization and application for removal of microcystin. J. Hazard. Mater. 2013, 252, 355-366.
(14) Harkins, A. L.; Duri, S.; Kloth, L. C.; Tran, C. D. Chitosan-cellulose composite for wound dressing material. Part 2. Antimicrobial activity, blood absorption ability, and biocompatibility. J. Biomed. Mater. Res. B 2014, 102, 1199-1206.
(15) Pelton, J. T.; McLean, L. R. “Spectroscopic Methods for Analysis of Protein Secondary Structure”, Anal. Biochem. 2000, 277, 167-176.
(16) Kuroda, Reiko; Honma, Takekiyo “CD spectra of solid-state samples”, Chirality, 2000, 12, 269-277.
(17) Kuroda, Reiko; Harada, Takunori “Solid State Chiroptical Spectroscopy: Principles and Applications” in Comprehensive Chiroptical Spectroscopy. Vol 1: Instrumentation, and Theoretical Simulations, Edited by N. Berova, Pl. L. Polavarapu, K. Nakanishi and R. W. Woody, John Wiley, 2012, pp 91-113.
(18) Oldfield, E. Chemical shifts and three-dimensional protein structures. J. Biomol. NMR 1995, 5, 217-225.
(19) Wishart, D. S.; Sykes, B. D. “the ¹³C chemical shift index: a simple method for the identification of protein secondary structure using ¹³C chemical shift data”, J. Biomol. NMR 1994, 4, 171-180.
(20) Dousseau, F.: and Pezolet, M. “Determination of the secondary structure content of proteins in aqueous solutions from their amide I and amide II infrared bands. Comparison between classical and partial least-squares methods”. Biochemistry 1990, 29, 8771-8779.
(21) Arrondo, J. L. R.; Muga, A.; Castresana, J.; Gofii, F. M. Quantitative studies of the structure of proteins in solution by Fourier-transform infrared spectroscopy. Prog. Biophys. Molec. Biol. 1993, 59, 23-56.
(22) Tamm, L. K.; Tatulian, S. A. Infrared spectroscopy of proteins and peptides in lipid bilayers. Quarterly Rev. Biophys. 1997, 30, 365-429.
(23) Dong, A.; Huang, P.; Caughey, W. S. Protein secondary structures in water from second-derivative amide I infrared spectra. Biochemistry 1990, 29, 3303-3308.
(24) S. Duri, S. Majoni, J. M. Hossenlopp, C. D. Tran, Determination of Chemical Homogeneity of Fire Retardant Polymeric Nanocomposite Materials by Near-infrared Multispectral Imaging Microscopy, Anal. Lett. 2010,43, 1780-1789.
(25) Cardamone, J. M. Investigating the microstructure of keratin extracted from wool: Peptide sequence (MALDI-TOF/TOF) and protein conformation (FTIR). J. Mol. Struct. 2010, 969, 97-105.
(26) Pelton, J. T.; McLean, L. R. Spectroscopic methods for analysis of protein secondary structure. Anal. Biochem. 2000, 277, 167-176.
(27) Wold, S.; Sjöström, M.; Eriksson, L. PLS-regression: a basic tool of chemometrics. Chemom. Intell. Lab. Syst. 2001, 58, 109-130.
(28) Arrondo, J. L. R.; Muga, A.; Castresana, J.; Gofii, F. M. Quantitative studies of the structure of proteins in solution by Fourier-transform infrared spectroscopy. Prog. Biophys. Molec. Biol. 1993, 59, 23-56.
(29) Tamm, L. K.; Tatulian, S. A. Infrared spectroscopy of proteins and peptides in lipid bilayers. Quarterly Rev. Biophys. 1997, 30, 365-429.
(30) Dong, A.; Huang, P.; Caughey, W. S. Protein secondary structures in water from second-derivative amide I infrared spectra. Biochemistry 1990, 29, 3303-3308.
(31) Kumosinski, T. F.; Unruh, J. J. Quantitation of the global secondary structure of globular proteins by FTIR spectroscopy: comparison with X-ray crystallographic structure. Talanta 1996, 43, 199-219.
(32) Westad, F.; Schmidt, A.; Kermit, M. Incorporating chemical band-assignment in near infrared spectroscopy regression models. J. Near Infrared Spectrosc. 2008, 16, 265-273.
(33) Westad, F.; Martens, H. Variable selection in near infrared spectroscopy based on significance testing in partial least squares regression. J. Near Infrared Spectrosc. 2000, 8, 117-124.
(34) Anderssen, E.; Dyrstad, K.; Westad, F.; Martens, H. Reducing over-optimism in variable selection by cross-model validation. Chem. Intell. Lab. Syst. 2006, 84, 69-74.
(35) Westad, F.; Afseth, N. K.; Bro, R. Finding relevant spectral regions between spectroscopic techniques by use of cross model validation and partial least squares regression. Anal. Chim. Acta 2007, 595, 323-327.
(36) Bernstein, F. C.; Koetzle, T. F.; Williams, G. J. B.; Meyer Jr, E. F.; Brice, M. D.; Rodgers, J. R.; Kennard, O.; Shimanouchi, T.; Tasumi, M. The Protein Data Bank: a computer-based archival file for macromolecular structures. Arch. Biochem. Biophys. 1978, 185, 584-591.
(37) Kabsch, W.; Sander, C. Dictionary of protein secondary structure: pattern recognition of hydrogen-bonded and geometrical features. Biopolymers 1983, 22, 2577-2637.
(38) Li, R.; Wang, D. Preparation of regenerated wool keratin films from wool keratin-ionic liquid solutions. J. Appl. Polym. Sci. 2013, 127, 2648-2653.
(39) Peplow, P. V.; Roddick-lanzilotta, A. D. Orthopaedic materials derived from keratin. U.S. Pat. No. 20,050,232,963, 2005.
(40) Sowa, M. G.; Wang, J.; Schultz, C. P.; Ahmed, M. K.; Mantsch, H. H. Infrared spectroscopic investigation of in vivo and ex vivo human nails. Vib. Spectrosc. 1995, 10, 49-56.
(41) Karaman, I.; Qannari, E. M.; Martens, H.; Hedemann, M. S.; Knudsen, K. E. B.; Kohler, A. Comparison of Sparse and Jack-knife partial least squares regression methods for variable selection. Chemom. Intell. Lab. Syst. 2013, 122, 65-77.
(42) Navea, S.; Tauler, R.; de Juan, A. Application of the local regression method interval partial least-squares to the elucidation of protein secondary structure. Anal. Biochem. 2005, 336, 231-242.
(43) Dickerson, M. B.; Sierra, A. A.; Bedford, N. M.; Lyon, W. J.; Gruner, W. E.; Mirau, P. A.; Naik, R. R. Keratin-based antimicrobial textiles, films, and nanofibers. J. Mater. Chem. B 2013, 1, 5505-5514.
(44) Aluigi, A.; Zoccola, M.; Vineis, C.; Tonin, C.; Ferrero, F.; Canetti, M. Study on the structure and properties of wool keratin regenerated from formic acid. Int. J. Biol. Macromol. 2007, 41, 266-273.
(45) Cardamone, J. M., Investigating the microstructure of keratin extracted from wool: Peptide sequence (MALDI-TOF/TOF) and protein conformation (FTIR). J. Mol. Struct. 2010, 969 (1), 97-105.
(46) Greve, T. M.; Andersen, K. B.; Nielsen, O. F. Penetration mechanism of dimethyl sulfoxide in human and pig ear skin: An ATR-FTIR and near-FT Raman Spectroscopic in vivo and in vitro study, Spectroscopy, 2008, 22, 405-417.
(47) Cilurzo. F.; Selmin, F.; Aluigi, A.; Bellosta, S. Regenerated keratin proteins as potential biomaterial for drug delivery, Pol. Adv. Tech. 2013, 24, 1025-1028.
(48) Yoshimizu, H.; Ando, I. Conformational characterization of wool keratin and S-(carboxymethyl) kerateine in the solid state by carbon-13 CP/MAS NMR spectroscopy. Macromolecules 1990, 23, 2908-2912.
(49) Dickerson, M. B.; Sierra, A. A.; Bedford, N. M.; Lyon, W. J.; Gruner, W. E.; Mirau, P. A.; Naik, R. R. Keratin-based antimicrobial textiles, films, and nanofibers. J. Mater. Chem. B 2013, 1, 5505-5514.
(50) Tanabe, T.; Okitsu, N.; Tachibana, A.; Yamauchi, K. Preparation and characterization of keratin-chitosan composite film. Biomaterials 2002, 23, 817-825.
(51) Hameed, N.; Guo, Q. Blend films of natural wool and cellulose prepared from an ionic liquid. Cellulose 2010, 17, 803-813.
(52) Persson, A. M.; Sokolowski, A.; Pettersson, C. Correlation of in vitro dissolution rate and apparent solubility in buffered media using a miniaturized rotating disk equipment: Part 1. Comparison with a traditional USP rotating disk apparatus. Drug Discov. Ther. 2009, 3, 104-113.

### Example 4—Facile Synthesis, Structure, Biocompatibility and Antimicrobial Property of Gold Nanoparticle Composites from Cellulose and Keratin

Reference is made to Tran et al., “Facile synthesis, structure, biocompatibility and antimicrobial property of gold nanoparticle composites from cellulose and keratin,” Journal of Colloid and Interface Science 510 (2018) 237-245, the content of which is incorporated herein by reference in its entirety.

Abstract

A novel, one-pot method was developed to synthesize gold nanoparticle composite from cellulose (CEL), wool keratin (KER) and chloroauric acid. Two ionic liquids, butylmethylimmidazolium chloride and ethylmethylimmidazolium bis(trifluoromethylsulfonyl)imide were used to dissolve CEL, KER and HAuCl₄. X-ray diffraction and X-ray photoelectron results show that Au³⁺ was completely reduced to Au⁰NPs with size of (5.5±1) nm directly in the composite with NaBH₄. Spectroscopy and imaging results indicate that CEL and KER remained chemically intact and were homogeneously distributed in the composites with Au⁰NPs. Encapsulating Au⁰NPs into [CEL+KER] composite make the composite fully biocompatible and their bactericide capabilities were increased by the antibacterial activity of Au⁰NPs. Specifically, the [CEL+KER+Au⁰NPs] composite exhibits up to 97% and 98% reduction in growth of antibiotic resistant bacteria such as vancomycin resistant Enterococcus and methicillin resistant S. aureus, and is not cytotoxic to human fibroblasts. While [CEL+KER] composite is known to possess some antibacterial activity, the enhanced antibacterial observed here is due solely to added Au⁰NPs. These results together with our previous finding that [CEL+KER] composites can be used for controlled delivery of drugs clearly indicate that the [CEL+KER+Au⁰NPs] composite possess all required properties for successful use as dressing to treat chronic ulcerous infected wounds.

Introduction

Gold nanoparticles (Au⁰NPs) have been the subject of intensive research in recent years, due to their intriguing optical, electrical, chemical and biochemical properties. For example, Au⁰NPs are reported to exhibit high antimicrobial activity against both gram-positive and gram-negative bacteria. They have also shown to be effective antiviral agent [1-6]. The size, morphology and stability of Au⁰NPs are known to strongly affect their antimicrobial and antiviral activity [1-7]. It is known that colloidal Au⁰NPs undergo coagulation and aggregation in solution, which, in turn, lead to changes in their size and morphology and hence their antibacterial and antiviral properties. As a consequence, intense efforts have been made to control the morphologies of Au⁰NPs. One possible remedy is to anchor the Au⁰NPs into a supporting material in order to prevent their coagulation and aggregation so that they can maintain their activity. In fact, Au⁰NPs have been encapsulated in various man-made polymers, and such systems have been reported to retain some of their antimicrobial activity [8-11]. For example, anchoring Au⁰NPs onto poly [2-(methacrylamido)-glycopyranose and poly [2-(methacryloxy)ethyl trimethylammonium iodide) have proved to be effective against a few bacteria [8-11]. Unfortunately, reported Au⁰NPs-encapsulated polymers are based mainly on man-made polymers [1-11]. As such they are not biocompatible, may exhibit some toxicity, and hence may not be used for biomedical applications. It is, therefore, of particular importance to develop a novel method to anchor Au⁰NPs onto composites made from biopolymers such as polysaccharide (cellulose (CEL)) and protein (keratin (KER)) as these composites are not only biocompatible but also sustainable as CEL and KER are the most abundant biorenewable biopolymers on the earth.

We have demonstrated recently that a simple ionic liquid (IL), butylmethylimmidazolium chloride ([BMIm⁺Cl⁻]), can dissolve both CEL and KER and by use of this IL as the sole solvent, we developed a simple, GREEN and totally recyclable method to synthesize [CEL+KER] composites just by dissolution without using any chemical modifications or reactions. Spectroscopy (FTIR, NIR, ¹³C CP-MAS-NMR) results indicate that there was no chemical alteration in the structure of CEL and KER. The [CEL+KER]composites obtained were found to retain unique properties of their components, namely, superior mechanical strength from CEL and controlled release of drugs by KER [12-17]. Because [BMIm⁺Cl⁻] can also dissolve metal salt such as silver chloride, it should be possible to use this IL as the solvent to synthesize [CEL+KER] composite which contains silver ions or silver nanoparticles. In fact, by use of [BMIm⁺Cl⁻] as the sole solvent, we have recently developed a novel method to synthesize composites containing CEL, KER and silver in the form of either ionic (Ag⁺) or Ag⁰ nanoparticle (Ag⁺ NPs or Ag⁰NPs) [18]. The [CEL+KER+AgNPs] composite was found to inhibit growth of various bacteria. Unfortunately, both [CEL+KER+Ag⁺ NPs] and [CEL+KER+Ag⁰NPs] composites are cytotoxic to human fibroblasts [18]. However, [CEL+KER+AgNPs] composite is biocompatible when its Ag⁰NPs concentration at or below 0.48 mmol [18]. It is, therefore, tempting to use this synthetic method to synthesize [CEL+KER] composite which contains gold nanoparticles. This is because, as described above, gold nanoparticles are relatively less toxic and much more biocompatible and, more importantly, can inhibit growth of different types of bacteria and viruses than those by silver nanoparticles. Unfortunately, since gold metal salt such as chloroauric acid is not soluble in [BMIm⁺Cl⁻], it is not possible to use the synthetic method for silver nanoparticle composites to prepare [CEL+KER+Au⁰NPs] composite.

The information presented clearly indicates that it is possible to use [CEL+KER] as a biocompatible composite to encapsulate Au⁰NPs. Such considerations prompted us to initiate this study which aims to develop a novel, green and one-pot synthesis to synthesize [CEL+KER+Au⁰NPs] composite. It will be demonstrated in this paper that because another simple IL, ethylmethylimmidazolium bis(trifluoromethylsulfonyl)imide ([EMIm⁺Tf₂N⁻]) can dissolve chloroauric acid and is mixable with [BMIm⁺Cl⁻], it was possible for us to develop a novel method in which both ILs, [BMIm⁺Cl⁻] and ([EMIm⁺Tf₂N⁻], were use as solvents to dissolve CEL, KER and HAuCl₄, respectively, to prepare [CEL+KER+Au³⁺] composite. The Au³⁺ was reduced to Au⁰NPs directly, in the composite by NaBH₄. Because the [CEL+KER+Au⁰NPs] composite obtained can prevent the Au⁰NPs from changing size and morphology as well as undergoing coagulation, it should, therefore, fully retain the unique property of the gold nanoparticles for repeated use without any complication of reduced activity and incomplete recovery after each use. The synthesis, characterization, and property of the composite, including its antimicrobial activity and biocompatibility will be reported in this communication.

Materials and Methods

Chemicals.

Microcrystalline cellulose (DPz300) and HAuCl₄ were purchased from Sigma-Aldrich and used as received. Raw (untreated) sheep wool, obtained from a local farm, was cleaned by Soxhlet extraction using a 1:1 (v/v) acetone/ethanol mixture at 80±3° C. for 48 h. The wool was then rinsed with distilled water and dried at 100±1° C. for 12 h [12-15]. 1-Methylimidazole, ethylimidazole and n-chlorobutane (both from Alfa Aesar, Ward Hill, Mass.) were distilled and subsequently used to synthesize [BMIm⁺Cl⁻] and [EMIm⁺Cl⁻]. The latter was converted to [EMIm⁺Tf₂N⁻] using method previously reported [19]. Nutrient broth (NB) and nutrient agar (NA) were obtained from VWR (Radnor, Pa.). Minimal essential medium (MEM), Fetal Bovine Serum (FBS), and Penicillin-Streptomycin were obtained from Sigma-Aldrich (St. Louis, Mo.), whereas PBS, and trypsin solution (Gibco) were obtained from Thermo Fischer Scientific (Waltham, Mass.). CellTiter 96® AQueous Non-Radioactive Cell Proliferation Assay was obtained from Promega (Madison, Wis.).

Bacterial and Cell Cultures.

The bacterial cultures of methicillin resistant Staphylococcus aureus (MRSA) ATCC 33591, vancomycin resistant Enterococcus faecalis (VRE) ATCC 51299, and the cell culture of human fibroblasts ATTC CRL-2522 were purchased from the American Type Culture Collection (ATCC, Rockville, Md.).

Synthesis.

[CEL+KER+Au⁰NPs] composites were synthesized with minor modification to those used for [CEL/CS+KER] composites [12-15,19]. As shown in Scheme 1, washed wool was dissolved in [BMIm⁺Cl⁻] at 120° C. Once dissolved, the solution temperature was reduced to 90° C. before CEL was added to the KER solution. Using this procedure, [BMIm⁺Cl⁻] solution of CEL and KER containing up to total concentration of 6 wt % (relative to IL) with various compositions and concentrations were prepared. Concurrently, in a separate flask, 240 mg of HAuCl₄ were dissolved in 2 mL of [EMIm⁺Tf₂N⁻], and the mixture was then added dropwise to the [BMIm⁺Cl⁻] solution of [CEL+KER]. The resulting solution was casted onto PTFE molds with desired thickness on Mylar films to produce thin composite film of [CEL+CS+ of Au³⁺]. They were then kept at room temperature for 24 hrs to allow gelation to yield Gel Films. The Gel Films were washed in 400 mL of 50:50 (v/v) THF:H₂O 50:50 for 24 hours to remove [EMIm⁺Tf₂N⁻], and then with water for 4-6 days to completely remove [BMIm⁺Cl⁻] to yield Wet Films. Washing water (2 L for a composite film of about 10 cm×10 cm) was repeatedly replaced with fresh water every 24 hrs until it was confirmed that IL was not detected in the washed water (by monitoring UV absorption of the IL at 290 nm). It was found that after washing for 72 hours, no IL was detected in the washing water by UV measurements. Since the limit of detection of the spectrophotometer used in this work was estimated to be about 3×10⁻⁵ AU, and the molar absorptivity of [BMIm⁺Cl⁻] at 290 nm is 2.6 M⁻¹ cm⁻¹, it is estimated that if any [BMIm⁺Cl⁻] remains, its concentration would be smaller than 2 μg/mL of the washed water and 2 μg/g of the composite film. Since this concentration is two orders of magnitude lower than the LD₅₀ value of the [BMIm⁺Cl⁻][20], if any IL remains in the composite films, it would not pose any harmful effect. Furthermore, as we have previously shown that results of UV-vis, FTIR and NIR techniques confirm that when the composite films were washed with water, [BMIm⁺Cl⁻] was removed from the films to a level not detectable by these techniques [12-20]. Subsequently, the Au³⁺ doped Wet Films were reduced with NaBH₄ to Au⁰NPs. For example, the Wet Film, sandwiched between two PTFE meshes, was placed in 400 mL of 20 mM of NaBH₄ in methanol at room temperature for 24 hrs. The reduced film was then washed and dried slowly (˜3-5 days) at room temperature in a humidity-controlled chamber to yield [CEL+KER+Au⁰NPs] composite.

Analytical Characterization.

FTIR spectra (from 450-4,000 cm⁻¹) were recorded on a Spectrum 100 Series FTIR spectrometer (Perkin Elmer, USA) at resolution of 2 cm⁻¹ by the KBr method. Each spectrum was an average of 64 individual spectra. X-ray diffraction (XRD) measurements were taken on a Rigaku MiniFlex II diffractometer utilizing the Ni filtered Cu Kα radiation (1.54059 Å). The voltage and current of the X-ray tube were 30 kV and 15 mA respectively. The samples were measured within the 20 angle range from 2.0 to 40.00. The scan rate was 5° per minute. Data processing procedures were performed with the Jade 8 program package [12-20]. X-ray photoelectron (XPS) spectra were taken on a HP 5950A ESCA spectrometer with Al monochromatic source and a flood gun used for charge suppression. The surface and cross-sectional morphologies of the composite films were examined under vacuum with a JEOL JSM-6510LV/LGS Scanning Electron Microscope with standard secondary electron (SEI) and backscatter electron (BEI) detectors. Prior to SEM examination, the film specimens were made conductive by applying a 20 nm gold-palladium-coating onto their surfaces using an Emitech K575x Peltier Cooled Sputter Coater (Emitech Products, TX).

In vitro antibacterial assay. The composites [CEL+KER+Au⁰NPs] were tested for potential antibacterial activity against antibiotic resistant bacteria such as methicillin resistant S. aureus (ATCC 33591) (MRSA) and vancomycin resistant Enterococcus faecalis (ATCC 51299) (VRE), using previously published protocol [12, 17, 18, 21]. Prior to the assays, cultures were grown overnight at 37° C. and 150 rpm. Composites were cut into 3×20 mm strips and autoclaved at 121° C., 15 psi for 20 min. The overnight cultures were diluted to 2 mL and put in contact with the material for 24 hours. Test tubes with bacteria not exposed to any composite served as a control, whereas bacteria exposed to [CEL+KER] without Au⁰NPs served as a blank. The tubes were incubated for 24 hours at 37° C. and 600 rpm. Before (time 0) and after the exposure (24 hours), the bacteria were diluted and plated onto nutrient agar plates, which were then incubated overnight at 37° C. Colony forming units (CFUs) were counted the next day and compared to the corresponding CFU numbers at time 0. The results were expressed as Log of reduction in number of bacteria, calculated as [log (N₀/N₂₄)], where N₀ is the number of CFUs at the beginning of the experiment, and N₂₄ is the number of bacteria after 24 hours). All experiments were carried out in triplicates; the variability between them was expressed as a standard error.

Biocompatibility assay. The biocompatibility of [CEL+KER+Au⁰NPs] composites was evaluated with the culture of human fibroblasts (ATTC CRL-2522) through 3 and 7 days as previously published [12,17,18, 21]]. The composites in shape of circles with 7 mm in diameter were prior to the experiment thermally sterilized at 121° C., 15 psi for 20 min. Human fibroblasts were grown in a sterile minimal essential medium (MEM) supplemented with 10% FBS and 1% Penicillin-Streptomycin according to ATCC guidelines, and incubated at 37° C. in a humified atmosphere of 5% CO₂ until the 3rd passage. Cells were seeded in a 24-well plate at a concentration of 2×10⁴ cells/mL as specified in guidelines for proliferation assay (Promega) and left for 1 day to allow for their attachment. The following day the sterilized composites were added to the wells and incubated with the cells for 3 and 7 days. Some wells did not contain composites and served as a control, whereas other wells contained [CEL+KER] composites without Au⁰NPs and served as a blank. After the incubation the viability and morphology of cells were evaluated with both, colorimetric CellTiter 96® AQueous Non-Radioactive Cell Proliferation Assay, and Olympus microscope camera with CellSens imaging software. The procedure for the CellTiter 96® AQueous One Solution Cell Proliferation Assay was followed as specified in the manufacturer's manual. In brief, the MTS reagent was added in a 1:5 ratio to each well after the medium in wells was supplemented with a colorless MEM. The cells were incubated at standard culture conditions for 3 h, and the optical density was measured with a Perkin Elmer HTS 7000 Bio Assay Reader at 490 nm. The percent viability was calculated using the following equation:

$$\% cell viability = \frac{{OD}_{Test sample}}{{OD}_{Control}} \times 100 \qquad ( 1 )$$

where OD_{Test Sample} is the measured OD of the test sample well, and OD_{Control} is the measured OD of the control well. Material was considered to be cytotoxic if viability of cells after the incubation was below 70% as specified in ISO 10993-5:2009(E) [22]. All experiments were carried out in triplicates; the variability between them was expressed as a standard error.

Results and Discussion

FT-IR.

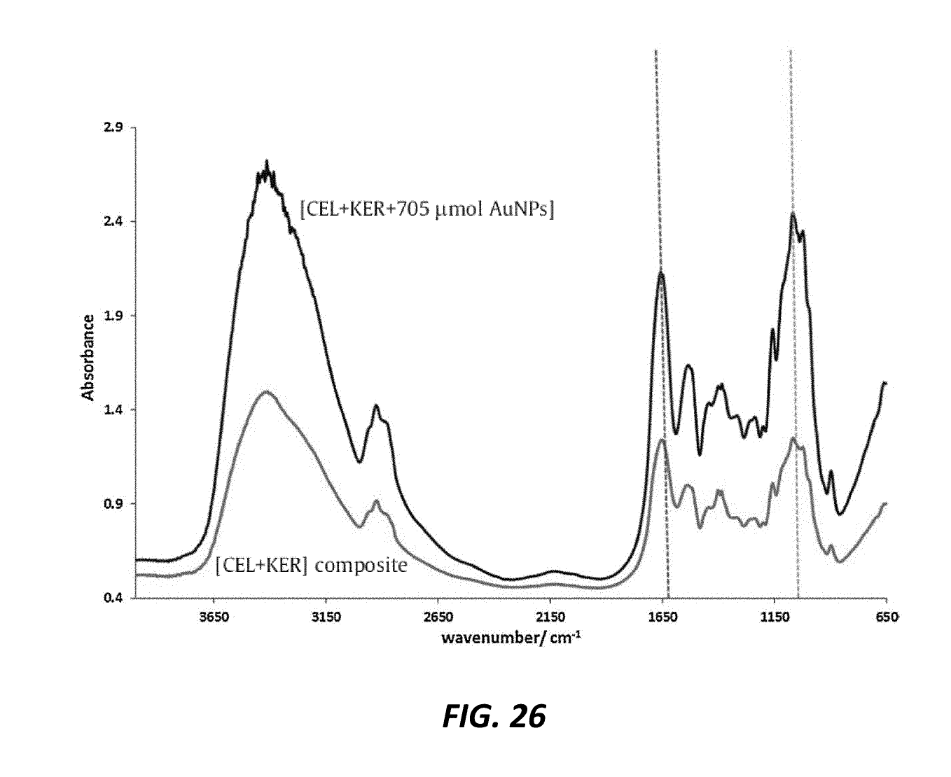

FTIR spectrum of the [CEL+KER+Au⁰NPs] composite is shown in FIG. 26. For reference, spectrum of the [CEL+KER] composite is also shown in FIG. 26. As expected, the spectrum of the [CEL+KER] composite is similar to those previously observed for [CEL+KER] composites, namely bands at ˜1650 cm⁻¹ and ˜1530 cm⁻¹ are due to amide C═O stretch (amide I) and C—N stretch (amide II) vibrations, and at 1300-1200 cm⁻¹ are from the in-phase combination of the N—H bending and the C—N stretch vibrations (amide III) [12-15]. Major bands between 1200- and 900-cm⁻¹ are due to sugar ring deformations of the CEL [12-15]. As shown, the spectrum of the [CEL+KER+Au⁰NPs] composite is relatively similar to the spectrum of the [CEL+KER] composite. It seems, therefore, that there may not be strong interaction between the Au⁰NPs and CEL and KER in the composite. However, careful inspection of the spectra, shown as four vertical dashed lines in the graph for clarification, reveals that there are, in fact, minor differences between the two spectra. It seems that interactions between Au⁰NP and C═O group lead to a shift in the intense amide band at 1646 cm⁻¹ and the smaller band at 1529 cm⁻¹ (of the [CEL+KER] composite) to 1649 cm⁻¹ and 1532 cm⁻¹ (of the [CEL+KER+Ag⁰NP] composite), respectively. Furthermore, bands due to CEL were also shifted when Au⁰NPs are present into the composite. Specifically, the sugar ring deformation band at 1062 cm⁻¹ in [CEL+KER] composite shifted to 1065 cm⁻¹, and the O—H band at 2919 cm⁻¹ shifted to 2023 cm⁻¹ when Au⁰NPs were added to the composite. These results suggest that KER and CEL may interact with Au⁰NP through the amide groups of the former, and the O—H groups of the latter [12-15].

Powder X-Ray Diffraction (XRD).

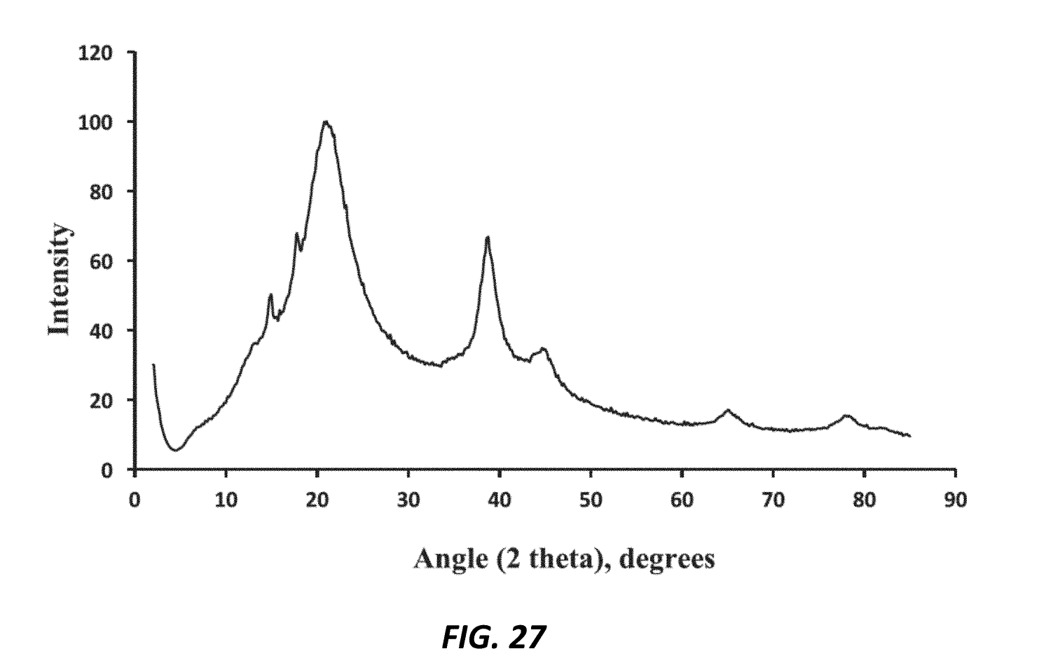

X-ray diffractogram of [CEL+KER+Au⁰NPs] composite is shown in FIG. 27. Because both CEL and KER are present in the composite, as expected, the diffractogram exhibits a large peak at around 2θ=21.30° which is due to CEL and KER. In addition to this peak, the diffractogram also has four peaks at (2θ)=36.78°, 44.56°, 65.06° and 78.05°. The fact that these peaks correspond well with Miller indices of (1 1 1), (2 0 0) and (2 0 0) and (3 1 1) of metallic gold nanoparticles confirms that Au³⁺ were successfully reduced to Au⁰ and present as Au⁰NPs in the composite [23-26].

Scherrer equation was then used to determine the size (t value) of the Au⁰ NPs in the composites from the full width at half maximum (FWHM, 0 value in the equation) of its corresponding XRD peaks [27,28].

$$τ = \frac{k λ}{βcosθ} \qquad ( 2 )$$

where τ is the size of the nanoparticle, X is the X-ray wavelength and k is a constant [27, 28]. The size of the metallic gold nanoparticle in the [CEL+KER+Au⁰] composite was found to be (5.5±1) nm.

Scanning Electron Microscope (SEM) Images and Energy Disperse Spectroscopy (EDS) Analysis.

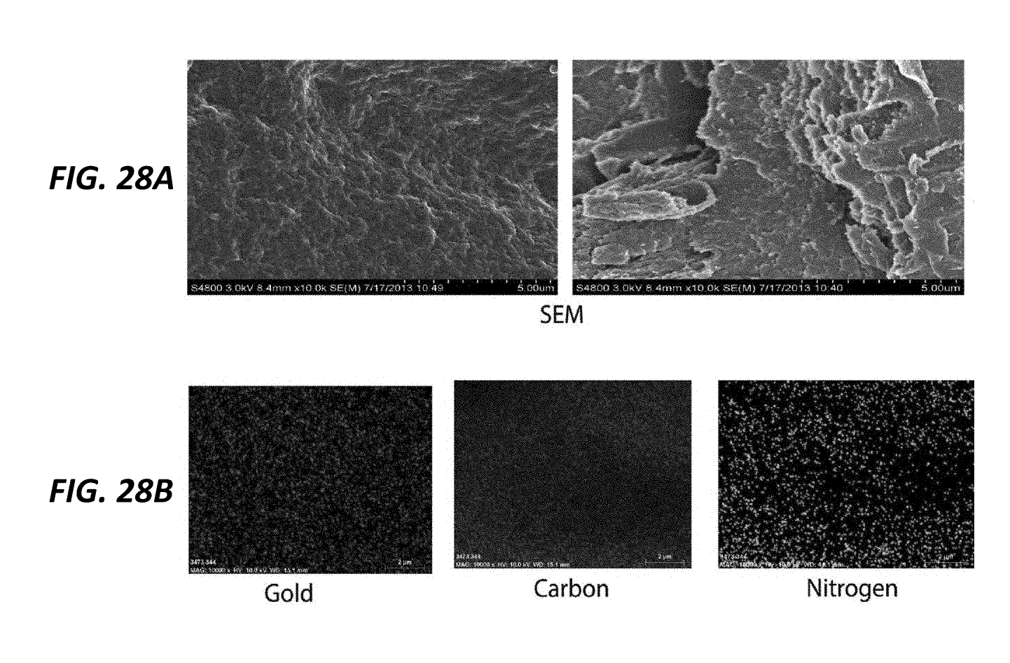

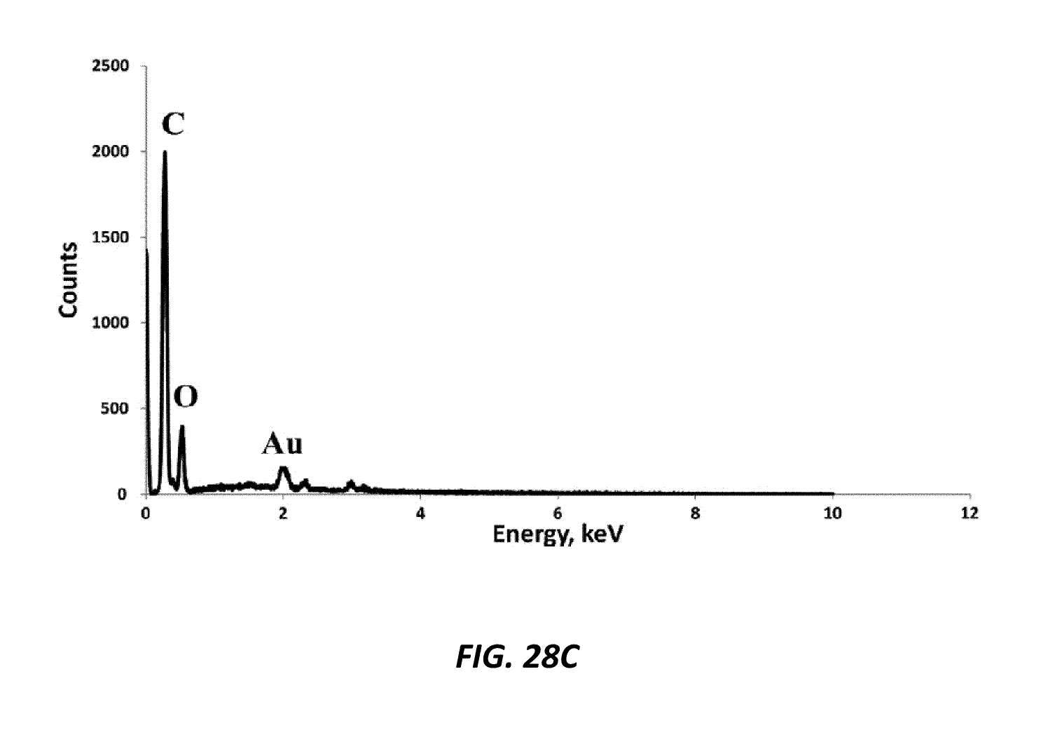

FIG. 28 shows the surface (left) and cross-sectional (right) SEM images of [CEL+KER+240 mg Au⁰NPs] composite. As expected, these images are similar to those reported previously for [CEL+KER] composite namely, the composite is homogenous, somewhat porous and has a rough surface [12-15]. This may be due to the fact that while CEL exhibits smooth and homogeneous morphology without any pores, KER is known to have a rough and porous structure with a three dimensional interconnection throughout the film surface [12-15]. This porous structure seems to reflect the physical properties of KER films such as its brittleness [12-15]. As a consequence, incorporating CEL into KER matrix results in a composite which is rough and porous. More information on the chemical composition and distribution of the Au⁰NPs can be found in FIGS. 28B and C. Three images shown in FIG. 28B, are EDS image recorded for gold (left), carbon (center) and nitrogen (right). It is evident from this images that not only CEL and KER but also Au⁰NPs homogenously distribute throughout the composite. The EDS spectrum (FIG. 28C) show that in addition to the two major bands at around 284 eV and 531 eV which are due to carbon and oxygen (of CEL and CS in composite) [17, 24], the third major band at ˜2 eV can be assigned to Au as this band is similar to those reported previously for gold [24].

X-Ray Photoelectron Spectroscopy (XPS).

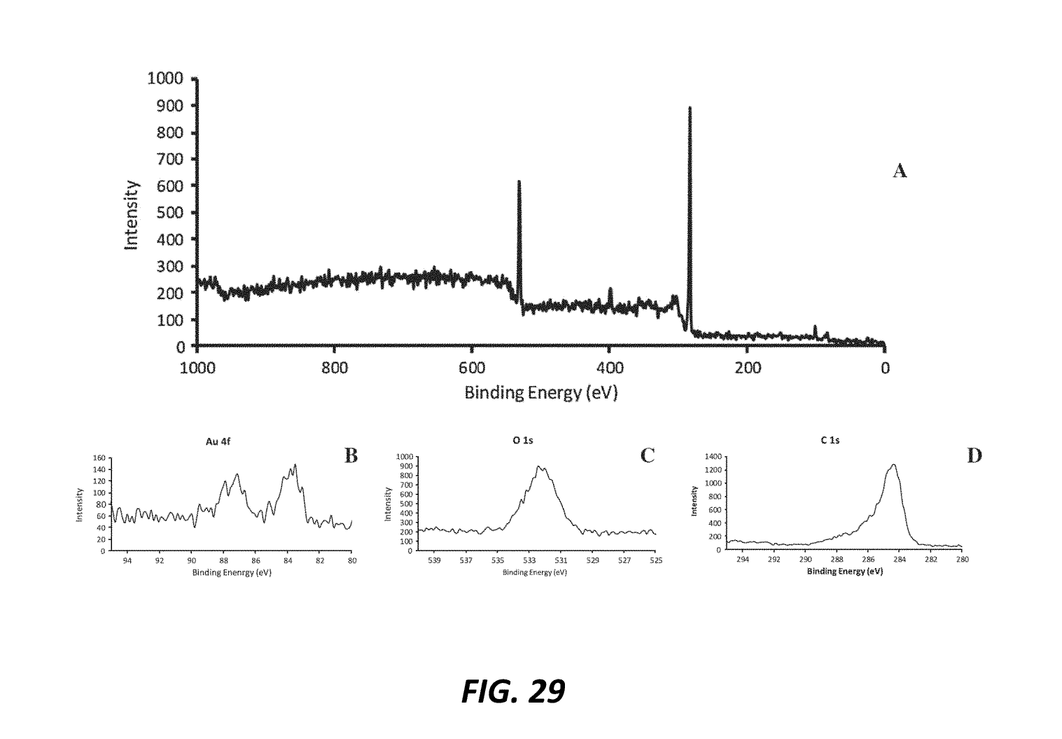

FIG. 29 shows the X-ray photoelectron of [CELsc+KER+705 μmol Au⁰NPs] composite. Two major bands at 284.4 eV and 532.0 eV, and their expanded view in FIGS. 29C and 29D, can be assigned to C is and O is, respectively [25, 29-32]. Since the content of gold in the composite is rather low, it is not surprising that its signal is not clear in FIG. 29A. However, as shown in FIG. 29B, when the region around 85 eV was magnified and expanded, a prominent doublet at 83.8 eV and 87.5 eV was clearly seen. Based on the fact that this doublet is characteristic of Au⁰ 4f_{7/2} and 4f_{5/2}, respectively, and the absence of any band due to Au³⁺ at around 86 eV indicates that all Au³⁺ was reduced to Au⁰ in the composite.

Antibacterial Assay.

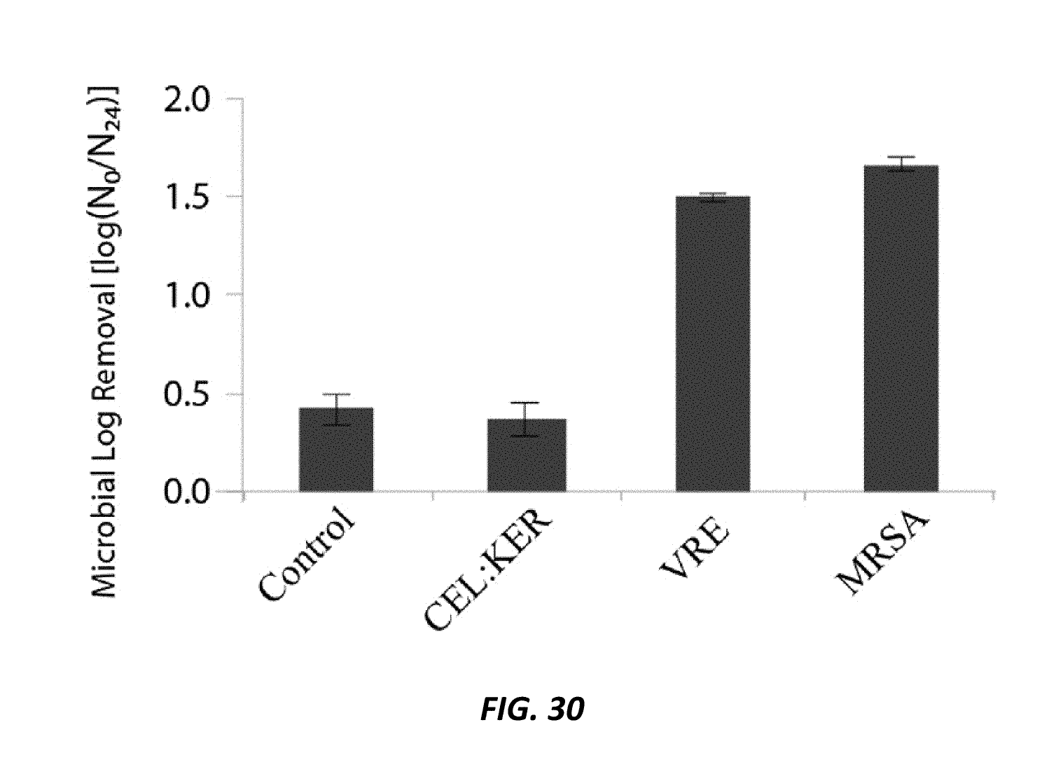

As described in the introduction, various Au⁰NPs encapsulated polymers have shown to be bactericide against both gram-positive and gram-negative bacteria such as E. coli, S. aureus, Shigella flexneri, Proteus mirabilis, Bacillus cereus and Bacillus subtilis [8-11]. However, to date, antimicrobial activity of Au⁰NPs-encapsulated composites/polymers against antibiotic resistant bacteria such as methicillin resistant S. aureus (MRSA) and vancomycin resistant Enterococcus (VRE) have not been investigated [8-11]. Since growth inhibition of such antibiotic resistant bacteria is of particular importance, we decided to investigate antimicrobial activity of the [CEL+KER+Au⁰NPS] against these bacteria. To assess the antimicrobial properties of the composite, the bacteria were grown in the presence of the composite and then plated out onto nutrient agar and measured by the number of colonies formed compared to those for the blank ([CEL+KER] composite) and the control (no material). Each assay was carried out three times. The results were calculated as microbial log of reduction and are shown in FIG. 30. It is evident from the figure that the [CEL+KER+Au⁰NPs] composite effectively and substantially inhibit growth of both antibiotic resistant bacteria VRE and MRSA. Specifically, up to (1.50±0.03) and (1.66±0.04) logs of reduction were found for VRE and MRSA, respectively, which correspond to 97 and 98% growth inhibition. It is important to point out that the antibacterial effect, which we report here, is due solely to the Au⁰NPs. As we have previously reported, [CEL+KER] composite also exhibits some antibacterial property [12-15]. However, because the antibacterial activities of the [CEL+KER+Au⁰NPs] composite reported here were compared to those of the blank (i.e., [CEL+KER] which is composite without Au⁰NPs) and the control (no composite), the reported bactericidal effect is entirely due to the Au⁰NPs.

Biocompatibility Assay.

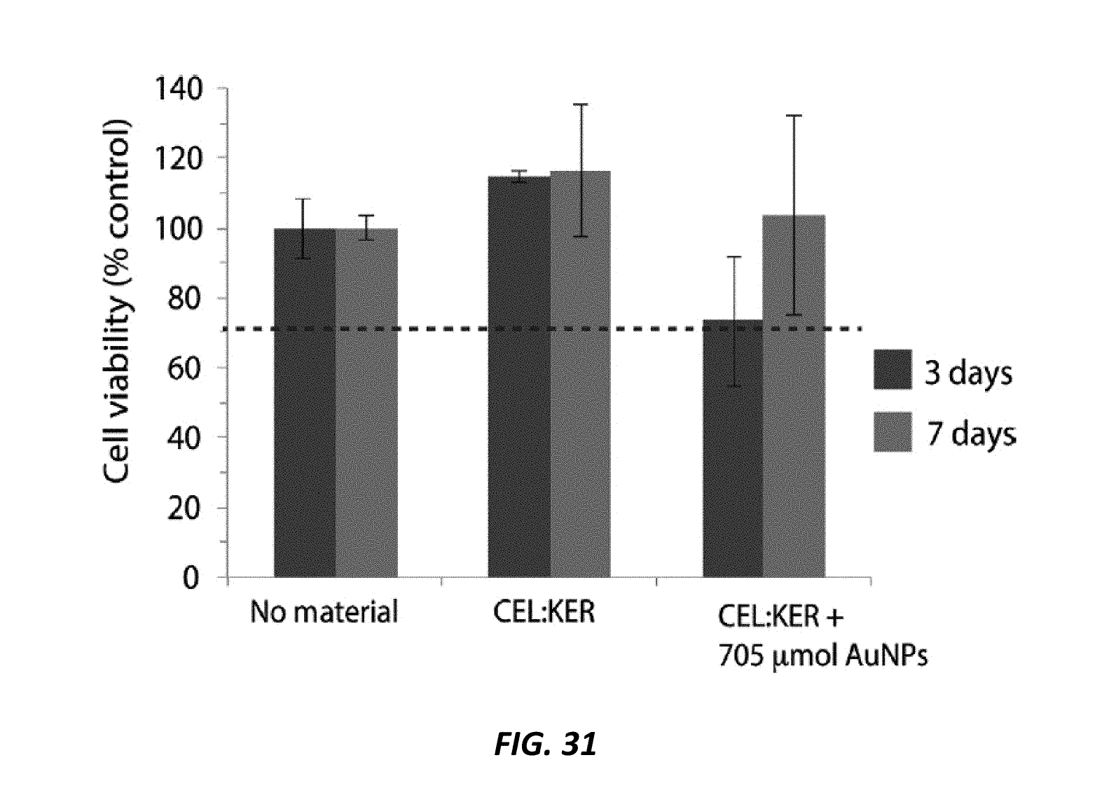

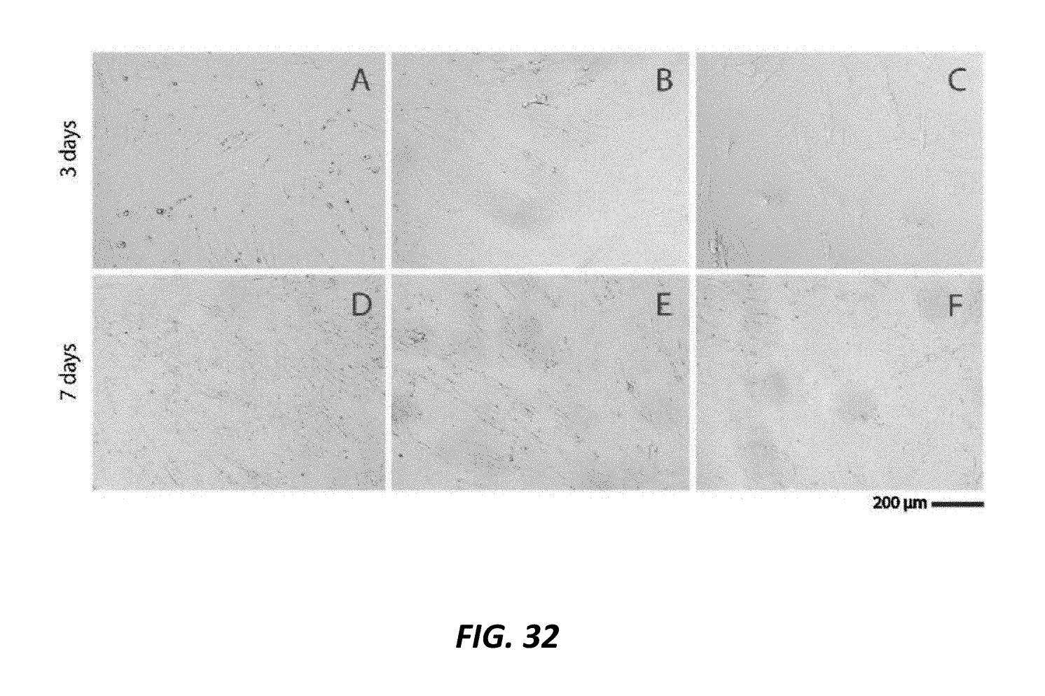

To assess a potential cytotoxicity of the [CEL+KER+Au⁰NPs] composite, the morphology and the proliferation capabilities of adherent human fibroblasts in presence and absence of biocomposites were analyzed. The proliferation capability was assessed using a colorimetric assay CellTiter 96® AQueous Non-Radioactive Cell Proliferation Assay, whereas the morphology of fibroblasts was examined microscopically. A material is considered to be cytotoxic if the viability of fibroblasts after the exposure was lower than 70% of control, as specified in ISO 10993-5: 2009(E) [22]. Fibroblasts were exposed to composites for 3 and 7 days. Viability of fibroblasts in the presence or absence of the [CEL+KER+Au⁰NPs] composite over time is shown in FIG. 31. Cells exposed to [CEL+KER+705 μmol Au⁰NPs] showed no statistically significant difference (at 95% confidence interval) compared to the control. Neither at 3 or 7 days the viability of cells dropped under 70%, which indicates that [CEL+KER+705 μmol Au⁰NPs] was not cytotoxic to human fibroblasts. Morphological data in FIG. 32 showed that the cells that were in contact with [CEL+KER+705 μmol Au⁰NPs] composite looked relatively healthy. After 3 days they exhibited an unusual morphology to some extent with thickened central part of their long bodies (FIG. 32C), but were still adherent, whereas after 7 days their morphology looked normal (FIG. 32F) and were not different from that of the cells in control and blank wells (FIGS. 32D&E).

Reports on biocompatibility of biopolymer-bound Au⁰NPs are rather limited whereas studies on colloidal Au⁰NPs report conflicting data regarding their biocompatibility [33-37]. For example, studies using a range of larger colloidal Au⁰NPs (30-90 nm) suggest their cytotoxicity is not size dependent [33-37], whereas others suggest that Au⁰NPs of smaller sizes (<15 nm) penetrate the plasma membrane and cause adverse effects to mammal cells [33-37]. In this study, we clearly and unequivocally demonstrate, for the first time, that any possible cytotoxicity of Au⁰NPs can be removed by incorporating them into the [CEL+KER+Au⁰NPs] composite. More importantly, the [CEL+KER+Au⁰NPs] composite is not only fully biocompatible but also fully retains its antimicrobial activity against antibiotic resistant bacteria such as VRE and MRSA.

It is of particular interest to compare the [CEL+KER+Au⁰NPs] composite to the [CEL+KER+AgNPs] composite which we reported recently [18]. We have shown that the silver nanoparticle composite exhibits strong antimicrobial activity against various bacteria including E. coli, S. aureus, Pseudomonas aeruginosa, VRE and MRSA, and its bactericide is correlated with the concentration of Ag⁰NPs in the composite. While the composite exhibits excellent antimicrobial activity at high Ag⁰NPs content, it is rather cytotoxic to human fibroblasts. Fortunately, at Ag⁰NPs of or below 480 μmol, the composite become biocompatible and still exhibit antibacterial. However, at Ag⁰NPs concentration of 480 μmol, the composite exhibits reduce growth of VRE and MRSA by only (1.04±0.08) and (0.28±0.08) logs of reduction, respectively, which correspond to 90% and 47% growth inhibition. Conversely, as expected, the [CEL+KER+Au⁰NPs] composite is not only bactericide but also is much more biocompatible. In fact, at Au⁰NPs concentration of 705 μmol which is 1.5× higher than the 480 μmol of Ag⁰NPs, the [CEL+KER+Au⁰NPs] is not only fully biocompatible but also exhibits stronger antimicrobial activity (97% and 98% against VRE and MRSA, respectively) compared to the [CEL+KER+480 μmol Ag⁰NPs] composite.

Conclusions

In summary, we have shown that gold nanoparticle composite was successfully and readily prepared from cellulose, wool keratin and chloroauric acid, in a simple one-pot synthesis in which two ionic liquids, [BMIm⁺Cl⁻] and [EMIm⁺Tf₂N⁻], were used as the solvent. XRD and XPS results show that Au³⁺ was completely reduced to Au⁰NPs with size of (5.5±1) nm directly in the composite with NaBH₄. FTIR results indicate that CEL and KER remain chemically intact in the composites. SEM and EDS measurements confirm that CEL, KER and Au⁰NPs were homogeneously distributed in the composites. Results of antimicrobial assays and biocompatibility show that encapsulating Au⁰NPs in this [CEL+KER] composite enables the composite to be fully biocompatible while extending the bactericidal effect of the [CEL+KER] composite by adding Au⁰NPs. Specifically, the [CEL+KER+Au⁰NPs] composite exhibits up to 97% and 98% reduction in growth of multidrug resistant bacteria such as VRE and MRSA, and is not cytotoxic to human fibroblasts. While [CEL+KER] composite is known to possess some antibacterial activity [13], the enhanced antibacterial observed here is due solely to added Au⁰NPs. This is because reported antibacterial activities are those of the [CEL+KER+Au⁰NPs] composite compared to [CEL+KER]. It is of particular interest to compare the [CEL+KER+Au⁰NPs] composite to the [CEL+KER+AgNPs] composite which we reported recently [18]. While the [CEL+KER+Ag⁰NPs] composite exhibits highly antimicrobial activity, it is rather cytotoxic to human at high Ag⁰NPs concentration. Because Au⁰NPs is relatively more biocompatible compared to Ag⁰NPs, the [CEL+KER+705 μmol Au⁰NPs] composite, which has Ag⁰NPs concentration 1.5× higher than Ag⁰NPs in [CEL+KER+480 μmol Ag⁰NPs] composite, was found not only fully biocompatible but also stronger antibacterial. These results together with our previous finding that [CEL+KER] composites can be used for controlled delivery of drugs such as ciprofloxacin [13] clearly indicate that the [CEL+KER+Au⁰NPs] composite possess all required properties for successfully used as high-performance dressing to treat chronic ulcerous infected wounds. Furthermore, because of unique properties of Au⁰NPs, this biocompatible [CEL+KER+Au⁰NPs] composite can also be potentially used for many other applications including biosensors, therapeutic agents, and other drug delivery systems. These are subject of our current intense investigation.

### REFERENCES

1. X. Yang, M. Yang, B. Pang, M. Vara, Y. Xia, Gold nanomaterials at work in biomedicine. Chem. Rev.115 (2015) 10410-10488.
2. W. C. W Chan, Y. Gogotsi, J. H. Hafner, P. T. Hammond, M. C. Hersam, M. A. Javey, C. R. Kagan, A. Khademhosseini, N. A. Kotov, S. H. Lee, H. Mohwald, P. A. Mulvaney, A. E. Nei, P. J. Nordlander, W. J. Parak, R. M. Penner, A. L. Rogach, R. E. Schaak, M. M. Stevens, A. T. S. Wee, C. G. Willson, P. S. A. Weiss, Year for nanoscience. ACS Nano, 8 (2014) 11901-11903.
3. B. Pelaz, S. Jaber, D. Jimenez de Aberastrui, V. Wulf, T. Aida, J. M. de la Fuente, J. Feldmann, H. E. Gaub, L. Josephson, C. R. Kagan, N. A. Kotov, L. M. Liz-Marzan, H. Mattoussi, P. Mulvaney, C. B. Murray, A. L. Rogach, P. S. Weiss, I. Willner, W. J. Parak, The state of nanoparticle-based nanoscience and biotechnology: progress, promises, and challenges. ACS Nano, 6 (2012) 8468-8483.
4. R. Sardar, J. S. Shumaker-Parry, Spectroscopic and microscopic investigation of gold nanoparticle formation: ligand and temperature effects on rate and particle size. J. Am. Chem. Soc. 133 (2011) 8179-8190.
5. A. R. Wright, M. Li, S. Ravula, M. Cadigan, B. El-Zahab, S. Das, G. A. Baker, I. M. Warner, Soft- and hard-templated organic salt nanoparticles with the midas touch: gold-shelled NanoGUMBOS. J. Mat. Chem. C, 2 (2014) 8996-9003.
6. Y. Takagai, R. Miura, A. Endo, W. L. Hinze, One-pot synthesis with in situ preconcentration of spherical monodispersed gold nanoparticles using thermoresponsive 3-(alkyldimethylammonio)-propyl sulfate zwitterionic surfactants. Chem. Commun. 52 (2016) 10000-10003.
7. S. Vijayakumar, S.; Ganesan, Gold nanoparticles as an HIV entry inhibitor. Curr. HIV Res., 10 (2012) 643-646.
8. C. Wang, Q. Cui, X. Wang, L. Li, Preparation of hybrid gold/polymer nanocomposites and their application in a controlled antibacterial assay, ACS Appl. Mater. Interfaces, 2016, 8 (42), 29101-29109.
9. Y. Yuan, F. Liu, L. Xue, H. Wang, J. Pan, Y. Cui, H. Chen, L. Yuan, Recyclable Escherichia coli-specific-killing Au⁰NP-polymer (ESKAP) nanocomposites, ACS Appl. Mater. Interfaces. 8 (2016) 11309-11317.
10. P. K. Mandapalli, S. Labala, S. Chawla, R. Janupally, D. Sriram, V. V. K. Venuganti, Polymer-gold nanoparticle composite films for topical application: evaluation of physical properties and antibacterial activity, Polym. Compos. (2015), PagesAhead of Print. DOI: 10.1002/pc.23885
11. S. Das, A. Pandey, S. Pal, H. Kolya, T. Tripathy, Green synthesis, characterization and antibacterial activity of gold nanoparticles using hydroxyethyl starch-g-poly (methylacrylate-co-sodium acrylate): a novel biodegradable graft copolymer, J. Mol. Liq. 212 (2015) 259-265.
12. M. Rosewald, F. Y. S. Hou, T. M. Mututuvari, A. L. Harkins, C. D. Tran, Cellulose-chitosan-keratin composite materials: synthesis and immunological and antibacterial properties. ECS Trans. 64 (2014) 499-505.
13. C. D. Tran, T. M. Mututuvari, Cellulose, chitosan and keratin composite materials. Controlled drug release. Langmuir, 31 (2015) 1516-1526.
14. C. D. Tran, T. M. Mututuvari, Cellulose, chitosan and keratin composite materials. Facile and recyclable synthesis, conformation and properties. ACS Sustainable Chem. Eng. 4 (2016) 1850-1861.
15. C. D. Tran, F. Prosenc, M. Franko, G. Benzi, Synthesis, structure and antimicrobial property of green composites from cellulose, wool, hair and chicken feather. Carbohydr. Polym., 151 (2016) 1260-1276.
16. C. D. Tran, S. Duri, A. L. Harkins, Recyclable Synthesis, Characterization and Antimicrobial Activity of Chitosan-based Polysaccharide Composite Materials, J. Biomed. Mat. Res. A, 101 (2007) 2248-2257.
17. T. M. Mututuvari, A. L. Harkins, C. D. Tran, Facile synthesis, characterization, and antimicrobial activity of cellulose-chitosan-hydroxyapatite composite material: a potential material for bone tissue engineering. J. Biomed. Mater. Res. A. 101 (2013) 3266-3277.
18. C. D. Tran, F. Prosenc, M. Franko, G. Benzi, One-Pot Synthesis of Biocompatible Silver Nanoparticle Composites from Cellulose and Keratin: Characterization and Antimicrobial Activity, ACS Appl. Mater. Interfaces (2016) 8, 34791-34801.
19. T. M. Mututuvari, M. Supramolecular biopolymeric composite materials: green synthesis, characterization and applications. Ph.D. Dissertation, Marquette University, Milwaukee, Wis., 2014.
20. Simon Duri, April Harkins, Anna Frazier, Chieu D. Tran, Composites Containing Fullerenes and Polysaccharides: Green and Facile Synthesis, Biocompatibility and Antimicrobial Activity, ACS Sustainable Chem. Eng., (2017) 5, 5408-5417.
21. A. L. Harkins, S. Duri, L. C. Kloth, C. D. Tran, Chitosan-cellulose composite for wound dressing material. Part 2. Antimicrobial activity, blood absorption ability, and biocompatibility. J. Biomed. Mater. Res. B 102 (2014) 1199-1206.
22. Biological evaluation of medical devices—Part 5: Tests for in vitro cytotoxicity. ISO 10993; The International Organization for Standardization: Geneva, Switzerland, 2009.
23. JCPDS No. 04-0784. The Powder Diffraction File™ (PDF); International Centre for Diffraction Data: Newton Square, Pa.
24. D. V. Leff, L. Brandt, J. R. Heath, Synthesis and characterization of hydrophobic, organically-soluble gold nanocrystals functionalized with primary amines, Langmuir. 12 (1996) 4723-4730.
25. G. Singaravelu, J. S. Arockiamary, V. G. Kumar, K. A. Govindaraju, Novel extracellular synthesis of monodisperse gold nanoparticles using marine alga, Sargassum wightii Greville, Colloids Surf. B, 57 (2007) 97-101.
26. S. Shiv Shankar, A. Ahmad, R. Pasrichaa, M. Sastry, Bioreduction of chloroaurate ions by geranium leaves and its endophytic fungus yields gold nanoparticles of different shapes, J. Mater. Chem., 13 (2003) 1822-1826.
27. P. Scherrer, Bestimmung der Grisse und der inneren Struktur von Kolloidteilchen mittels Rintgenstrahlen, Nachr. Ges. Wiss. Gittingen. 1918 (1918) 98-100.
28. J. J. Langford, A. J. C. Wilson, Scherrer after sixty years: a survey and some new results in the determination of crystallite size, J. Appl. Crystallogr. 11 (1978) 102-113.
29. M. Brust, M. Walker, D. Bethell, D. J. Schiffrin, R. Whyman, Synthesis of thiol-derivatised gold nanoparticles in a two-phase liquid-liquid system, Chem. Commun. (1994) 801-802.
30. A. Kumar, S. Mandal, P. R. Selvakannan, R. Pasricha, A. B. Mandale, M. Sastry, Investigation into the interaction between surface-bound alkylamines and gold nanoparticles, Langmuir. 19 (2003) 6277-6282.
31. M. Conte, C. J. Davies, D. J. Morgan, T. E. Davies, A. F. Carley, P. Johnston, G. J. Hutchings, Modifications of the metal and support during the deactivation and regeneration of Au/C catalysts for the hydrochlorination of acetylene, Catal. Sci. Technol., 3 (2013) 128-134.
32. C. Sámano-Valencia, G. A. Martínez-Castañón, F. Martínez-Gutiérrez, F. Ruiz, J. F. Toro-Vázquez, J. A. Morales-Rueda, L. F. Espinosa-Cristóbal, N. V. Zavala Alonso, N. Niño Martínez, Characterization and biocompatibility of chitosan gels with silver and gold nanoparticles, J. Nanomater. 2014 (2014) 1-11.
33. A. Avalos, A. I. Haza, D. Mateo, P. Morales, Effects of silver and gold nanoparticles of different sizes in human pulmonary fibroblasts, Toxicol. Mech. Methods. 25 (2015) 287-295.
34. D. Mateo, P. Morales, A. Avalos, A. I. Haza, Comparative cytotoxicity evaluation of different size gold nanoparticles in human dermal fibroblasts, J. Exp. Nanosci. 10 (2015) 1401-1417.
35. N. Pernodet, X. Fang, Y. Sun, A. Bakhtina, A. Ramakrishnan, J. Sokolov, A. Ulman, M. Rafailovich, Adverse effects of citrate/gold nanoparticles on human dermal fibroblasts, Small. 2 (2016) 766-773.
36. T. Mironava, M. Hadjiargyrou, M. Simon, V. Jurukovski, M. H. Rafailovich, Gold nanoparticles cellular toxicity and recovery: effect of size, concentration and exposure time, Nanotoxicology. 4 (2010) 120-137.
37. Y. Pan, S. Neuss, A. Leifert, M. Fischler, F. Wen, U. Simon, G. Schmid, W. Brandau, W. Jahnen-Dechent, Size-dependent cytotoxicity of gold nanoparticles, Small. 3 (2007) 1941-1949.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An ionic liquid composition comprising a structural polysaccharide and a structural protein dissolved in an ionic liquid.

2. The composition of claim 1, wherein the structural polysaccharide is a polymer comprising 6-carbon monosaccharides linked via beta-1,4 linkages.

3. The composition of claim 1, wherein the structural polysaccharide comprises cellulose.

4. The composition of claim 1, wherein the structural polysaccharide comprises chitosan.

5. The composition of claim 1, wherein the structural protein comprises keratin.

6. The composition of claim 1, wherein the structural polysaccharide comprises cellulose and/or chitosan and the structural protein comprises keratin.

7. The composition of claim 1, comprising a combination of cellulose, chitosan, and keratin.

8. The composition of claim 1, further comprising metal nanoparticles and/or metal oxide nanoparticles.

9. The composition of claim 8, wherein the metal nanoparticles comprise gold, silver, or copper nanoparticles and/or wherein the metal oxide nanoparticles comprise gold, silver, or copper oxide nanoparticles.

10. The composition of claim 1, wherein the ionic liquid is an alkylated imidazolium salt.

11. The composition of claim 1, wherein the ionic liquid composition comprises at least 6% w/w of the dissolved structural polysaccharide and the dissolved structural protein.

12. A method for preparing a composite material comprising a structural polysaccharide, a structural polypeptide, and optionally metal nanoparticles and/or metal oxide nanoparticles, the method comprising preparing a ionic liquid composition according to claim 1 and removing the ionic liquid from the ionic liquid composition to retain the composite material.

13. The method of claim 12, comprising: (a) first dissolving the structural protein in the ionic liquid to prepare an ionic liquid composition comprising the structural protein, and (b) subsequently adding the structural polysaccharide to the ionic liquid composition and dissolving the structural polysaccharide to obtain an ionic liquid composition comprising the structural protein and the structural polysaccharide, and (c) subsequently removing the ionic liquid composition to retain the composite material comprising the structural protein and the structural polysaccharide.

14. The method of claim 13, wherein in step (a) the structural protein is dissolved at a temperature of about 110° C.-130° C. to prepare the ionic liquid composition comprising the structural protein and the temperature of the ionic liquid composition is reduced to about 80° C.-100° C. prior to performing step (b).

15. The method of claim 13, wherein the composite material comprises metal oxide nanoparticles and the method further comprises contacting the metal oxide nanoparticles with a reducing agent.

16. The method of any of claim 13, wherein the ionic liquid is removed by steps that include washing the ionic liquid composition with an aqueous solution to obtain the composite material and drying the composite material thus obtained.

17. A composite material prepared by the method of any of claim 13.

18. A method for killing or eliminating microbes, the method comprising contacting the microbes with the composite material of claim 17.

19. A filter comprising the composite material of claim 17.

20. A bandage comprising the composite material of claim 17.
